(19) United States
(12) Patent Application Publication
(10) Pub. No.: US 2024/0043941 A1
(43) Pub. Date: Feb. 8, 2024

(54) SYSTEMS, METHODS, AND APPARATUSES FOR CONCENTRATION AND IDENTIFICATION OF A MICROORGANISM FROM BLOOD

(71) Applicants: BioFire Diagnostics, LLC, Salt Lake City, UT (US); bioMerieux, Inc., Durham, NC (US)

(72) Inventors: Elizabeth Mary Ott Crowther, Sandy, UT (US); Marta Elizabeth Schaub, Bountiful, UT (US); Edward Preston Rich, Layton, UT (US); Andrew Carter Hatch, West Jordan, UT (US); Stephanie Anne Thatcher, Salt Lake City, UT (US); Ryan T. Hill, Durham, NC (US); Christopher S. Ronsick, Durham, NC (US); Mark S. Wilson, Hillsborough, NC (US); John D. Walsh, Bahama, NC (US); Kirk M. Ririe, Salt Lake City, UT (US); Clark L. Roper, Highland, UT (US)

(21) Appl. No.: 18/265,927

(22) PCT Filed: Dec. 14, 2021
(86) PCT No.: PCT/US2021/063288

§ 371 Date: Jun. 7, 2023

Related U.S. Application Data
(60) Provisional application No. 63/126,041, filed on Dec. 16, 2020.

(51) Int. Cl. C12Q 1/689; C12Q 1/6895; C12Q 1/6806; C12Q 1/24

(52) U.S. Cl. CPC C12Q 1/689; C12Q 1/6895; C12Q 1/6806; C12Q 1/24

(57) ABSTRACT

Systems, methods, and apparatuses for isolating and identifying a microorganism from a sample known to contain or that may contain a microorganism.

### RELATED APPLICATIONS

This application claims the benefit of and priority to U.S. Prov. Pat. App. No. 63/126,041 filed 16 Dec. 2020, the entirety of which is incorporated herein by reference.

### BACKGROUND

### 1. Technical Field

Embodiments of the present disclosure relate generally to systems, methods, and apparatuses for sepsis diagnosis direct from blood.

### 2. Background

In the United States, Canada, and Western Europe infectious disease accounts for approximately 7% of human mortality, while in developing regions infectious disease accounts for over 40% of human mortality. Infectious diseases lead to a variety of clinical manifestations. Among common overt manifestations are fever, pneumonia, meningitis, diarrhea, and diarrhea containing blood. While the physical manifestations suggest some pathogens and eliminate others as the etiological agent, a variety of potential causative agents remain, and clear diagnosis often requires a variety of assays be performed.

In the US, bloodstream infection (BSI) and resulting septic shock (also referred to as sepsis, septicemia, bacteremia, fungemia, candidiasis, candidemia, bloodborne infection, and other related terms) is a leading cause of death. For instance, bacterial BSI is the 11th leading cause of death amongst adults and 7th amongst infants. Candida spp. and other fungi can also cause BSI. Bloodstream infections of Candida spp. are associated with a high mortality rate (40%), which is mainly attributed to the long diagnostic time required by blood culture. Studies have shown that initiation of appropriate antibacterial or antifungal treatment can reduce mortality rates and that for every hour of delay in antimicrobial administration significant increases in mortality are observed. Thus, early detection and definitive diagnosis and rapid treatment with appropriate antibiotics or antifungals are desired for improving outcome in patients with suspected BSI.

The current diagnostic gold standard for BSI requires growth of the organism in culture followed by microscopic observation, subculturing, and phenotypic identification of the purified isolate. This results in a reporting time ranging from 36-72 hours for Gram positive bacteria, 48-96 hours for Gram negative bacteria, and 48-120 hours for fungal infection. Alarmingly, approximately one-third of the patients who are treated for fungal BSI never show positive blood culture growth and many positive cases of fungal BSI are definitively diagnosed based only upon post-mortem analysis. As a result, it is typical for physicians to begin treating patients suspected of having BSI with a regimen of broad-spectrum antibiotics or antifungals immediately after drawing blood for culture. This is not ideal. Studies have shown that administration of inadequate or ineffective antimicrobial treatment does not help to improve patient outcomes and, distressingly, has been found to lead to the rise of drug-resistant organisms, which independently hurts patient outcomes and public health as a whole.

One alternative to the diagnostic gold standard (i.e., classical microbiological methods) includes molecular identification of infectious bacteria and fungi from blood. However, the number of infectious organisms found in whole blood in BSI is usually low (˜1-100 colony-forming units per milliliter of blood (cfu/ml) with ˜1-10 cfu/ml being typical in most individuals with culture-confirmed sepsis). Moreover, blood contains a number of inhibitors of the Polymerase Chain Reaction (PCR) (e.g., hemoglobin and other blood proteins (e.g., human serum albumin) and genomic DNA from white blood cells that can co-purify with microorganisms and interfere with both nucleic acid recovery from the target microorganisms and downstream PCR). With so few organisms in whole blood and the presence of PCR inhibitors, concentrating from larger volumes of whole blood (e.g., 1-20 mL) is desired to obtain the quality and quantity of DNA template desired to achieve sensitivity at clinically relevant microorganism levels.

There is an urgent need for more rapid, accurate molecular-based diagnostics to reduce the number of doses of ineffective or unnecessary broad-spectrum antimicrobials received by uninfected patients. Rapid diagnostics can allow for the timely administration of a more tailored and effective antimicrobial therapy to those who do have a BSI. Despite these many potential advantages, many of the rapid BSI diagnosis solutions that have been tried have not been widely adopted. This is for a variety of reasons including cumbersome workflows, time to result, and cost.

One product on the market is called MolYsis™, which offers the promise of selective isolation of bacterial DNA from intact organisms in whole blood. The MolYsis™ Complete5 DNA extraction kit (Cat #D-321-100; Molzym GmbH & Co. KG, Bremen, Germany) includes a chaotropic lysis buffer for selective lysis of blood factors (red blood cells, white blood cells, etc.) and DNase for degradation of genomic DNA. Microorganism cells are recovered by centrifugation, the supernatant is discarded, the cells are resuspended and repelleted several times in different buffers, chemical lysis of microbial cells is performed, and finally microorganism nucleic acids are recovered. In all, the MolYsis™ kit involves a cumbersome workflow that takes ˜45 minutes for sample preparation plus another ˜45 minutes for microorganism cell cleanup and lysis. Identification of microorganisms requires inputting the nucleic acids recovered with the MolYsis™ kit into another assay, which takes additional time and involves additional expense. In addition, successful use of the MolYsis™ kit requires a skilled technician. The dependence on skill of the operator raises the risk of operator-to-operator differences in yield and quality of results. The many buffers and manual pipetting steps increases the risk of cross-contamination of samples.

Another product that is intended to be used to identify BSI from whole blood is T2MR® from T2 Biosystems. The T2MR® system includes automated sample preparation that includes selective lysis of blood factors, microorganism recovery, microorganism lysis, recovery of microorganism-derived nucleic acids, and PCR amplification. The T2MR® system uses nuclear magnetic resonance (NMR) for microorganisms identification. Superparamagnetic NMR nanoprobes in solution bind to microorganism-specific DNAs and form agglomerates that can be detected by the NMR. Nanoprobes agglomerated by the presence of microorganism nucleic acids yield a greater NMR signal as compared to the signal from unagglomerated nanoprobes. However, the T2MR® system requires 4-6 hours of NMR data collection in order to obtain data of sufficient quality that can be used for microorganism identification. The T2MR® system is also expensive (the instrument costs about $150,000) and the throughput for the instrument is severely limited due to the time required for data collection. In addition, bacteria and fungi associated with BSI are tested on separate T2MR® panels. This means that a patient presenting with sepsis symptoms would have to be tested against the bacterial and fungal panels in order to rule in/rule out bacterial and fungal causes. In addition, the numbers of organisms tested on the bacterial and fungal panels are limited (about six organisms are tested on each) and the tests provide no information about drug susceptibility/resistance.

The present invention addresses various improvements relating to identification of BSI-associated microorganisms directly from blood with a simplified workflow and more rapid sample-to-answer.

### BRIEF SUMMARY

The present invention provides methods, systems, and apparatuses for concentrating, characterizing and/or identifying microorganisms from a sample. In one embodiment, the microorganism is a bacterium. In another embodiment, the microorganism is fungal organism (e.g., a yeast or mold). In a further embodiment, the microorganism is a parasite. The methods, systems, and apparatuses may be particularly useful for the separation, concentration, characterization and/or identification of microorganisms from complex samples such as blood or urine or cerebrospinal fluid. In a preferred aspect, the methods, systems, and apparatuses of the present invention may be used for concentrating, characterizing and/or identifying microorganisms direct from whole blood in order to rapidly determine that a patient is septic. In typical sepsis, the concentration of microorganisms in the blood stream is low. E.g., ˜<1-100 cfu/ml, with ˜<1-10 cfu/ml being typical. In septic patients or patients suspected of being septic, the microorganisms in blood, if they are present, are too dilute to be identified directly from a blood sample without the methods described herein. Moreover, blood contains a number of inhibitors of PCR (e.g., hemoglobin, human serum albumin and genomic DNA) that suitably may be removed for consistently successful identification and analysis of microorganisms from whole blood and other complicated matrices (e.g., urine and CSF). The present invention provides methods, systems, and apparatuses for selective lysis of non-microbial cells in a sample and concentration of microorganism from a relatively large volume (e.g., 10-20 ml) of sample. In a preferred embodiment, the methods, systems, and apparatuses described herein do not include use of devices or steps such as, but not limited to, mixing the blood sample and the differential lysis buffer in a first container and then transferring the lysate to the centrifugal concentrator, including components other than the blood sample and the differential lysis buffer in the centrifugal concentrator, opening the centrifugal concentrator to decant a supernatant fraction after centrifugation, recovery of the microorganism by centrifugation with a density cushion or a physical separator, pretreating the blood sample (other than mixing the blood sample with a differential lysis buffer and proceeding with the concentration and identification steps described in the methods herein), a pre-analysis culturing step, a step of subculturing the sample to identify the microorganisms present in the sample, or a DNase step to digest non-microbial DNA from the selectively lysed non-microbial cells.

The invention described herein suitably may include a method of isolating and identifying a microorganism is described. The method suitably may include steps of (a) providing a volume of a blood sample suspected of containing the microorganism; (b) mixing the blood sample with a differential lysis buffer to yield a lysate, wherein the lysate comprises lysed blood cells and unlysed microorganism; (c) concentrating the microorganism from the lysate; (d) adding the microorganism to a device that includes one or more reagents needed for identifying the microorganism; and (e) identifying the microorganism present in the blood sample. In the method, the microorganism, if present, is concentrated in a range of 25 to 100 fold relative to the volume of the provided blood sample, and the microorganism, if present, has a concentration in a range of about <1 CFU/ml (but greter than zero) to about 100 CFU/ml in the provided blood sample (e.g., <1 CFU/ml 10 about 10 CFU/ml).

Method steps (a)-(c) suitably may be completed in a time range of about 10 to 20 minutes. Method steps d) and (e) suitably may be completed in a time range of less than 4 hrs, less than 3 hrs, less than 2 hrs, or less than 1 hr.

The microorganism in the method suitably may include one or more of a bacterium or fungal organism associated with a bloodborne infection.

The identifying in the method suitably may include one or more of a molecular test, a phenotypic test, a proteomic test, an optical test, or a culture-based test. The identifying suitably may include steps of isolating from the microorganism one or more nucleic acids characteristic of the microorganism, and analyzing the one or more nucleic acids to identify the microorganism present in the blood sample. In one embodiment of the foregoing method, the identifying further comprises amplifying one or more nucleic acids and then detecting the one or more amplified nucleic acids. Detecting the one or more amplified nucleic acids suitably may include use of one or more of a dsDNA binding dye, real-time PCR, a post-amplification nucleic acid melting step, a nucleic acid sequencing step, a labeled DNA binding probe, or an unlabeled probe. The steps of identifying suitably may be completed in a time range of about 5 to 75 minutes.

The method suitably may further include performing a culture step on the concentrated microorganism in culture media to increase concentration of the microorganism and then performing the steps of identifying, wherein the culture step is performed for 4 hrs or less, 3 hrs or less, or 2 hrs or less, 1 hr or less, 30 minutes or less, 20 minutes or less, or 10 minutes or less, preferably 3 hrs or less.

The differential lysis buffer used in the recited method suitably may include a buffering substance, a nonionic surfactant, a salt, and a pH range of about 10-11 prior to mixing the blood sample with the differential lysis buffer. The differential lysis buffer suitably may have a pH of about 7.0 to 8.0 after mixing the blood sample and the differential lysis buffer. The buffering substance used in the differential lysis buffer suitably may be selected from the group consisting of CABS, CAPS, CAPS, CHES, and combinations thereof. The buffering substance used in the differential lysis buffer suitably may be CAPS. The pH of the differential lysis buffer mixed with the blood sample suitably may be about 1.5 to 2.5 pH units below the pH buffering range of the buffering substance. The nonionic surfactant used in the differential lysis buffer suitably may be a polyoxyethylene (POE) ether, preferably one or more of Arlasolve 200 (aka, Poly(Oxy-1,2-Ethanediyl)), Brij O10, and nonaethylene glycol monododecyl ether (aka, Brij 35). The nonionic surfactant used in the differential lysis buffer suitably may be selected from the group consisting of Triton X-114, NP-40, Arlasolve 200, Brij O10 (aka, Brij 96/97), octyl β-D-glucopyranoside, a saponin, nonaethylene glycol monododecyl ether (aka, Brij 35), and combinations thereof. In the differential lysis buffer combined with a blood sample, the concentration of detergent (e.g., in a range of 0.1% to 0.5%) and pH (e.g., in a range of 7-11) suitably may be adjusted to minimize pellet volume while maximizing differential lysis of blood cells in the sample. Suitably the pellet volume may be less than or equal to ˜500 μL, less than or equal to ˜400 μL, less than or equal to ˜300 μL, less than or equal to ˜200 μL, or less than or equal to ˜100 μL. Suitably up to 50%, 60%, 70%, 80%, 90%, 95%, 97%, 98%, 99% on non-microbial cells in the sample may be lysed within 2-5 minutes of combining the sample with the differential lysis buffer.

Concentrating the microorganism from the lysate suitably may include centrifugation, and the concentrating further comprises recovering a pellet fraction comprising the microorganism from a supernatant fraction comprising a lysed blood fraction. Concentrating the microorganism from the lysate suitably may include disposing the blood sample mixed with the differential lysis buffer into a centrifugal concentrator, wherein the centrifugal concentrator comprises: a chamber having an opening at a first end and a seal portion at a second end, wherein the seal portion is configured to seal a second opening at the second end of the chamber; and a plunger movably disposed at least partially inside the chamber, wherein the plunger is configured to be actuated to open the seal portion; centrifuging the centrifugal concentrator to concentrate the microorganism from the blood sample disposed within the chamber; and expressing the concentrated microorganism from the second opening at the second end of the chamber. Expressing the concentrated microorganism from the second opening at the second end of the chamber suitably may include aseptically expressing the pellet from the second end of the centrifugal concentrator into a vial or an assay device. The centrifugal concentrator suitably may not include a density cushion or a physical separator for separating the microorganism from the lysate.

The method suitably may not include one or more of mixing the blood sample and the differential lysis buffer in a first container and then transferring the lysate to the centrifugal concentrator, including components other than the blood sample and the differential lysis buffer in the centrifugal concentrator, opening the centrifugal concentrator to decant a supernatant fraction after centrifugation, a culture step prior to mixing the blood sample with the differential lysis buffer, or a DNase step to digest genomic DNA in the lysate. The method suitably may not include one or more of a culture step prior to mixing the blood sample with the differential lysis buffer, or a DNase step to digest genomic DNA in the lysate.

The microorganism suitably may be concentrated from the lysate by a filtration technique. The method may suitably further include adding a filter with concentrated microorganism thereon to one or more of a culture apparatus or an assay device configured for identifying the microorganism present in the blood sample.

Suitably, the method steps of mixing the blood sample with the differential lysis buffer, yielding the lysate, and separating the microorganism from the lysate may be accomplished in a single tube. Suitably, the differential lysis buffer used in the method may be a single buffer provided in the single tube. Suitably, the differential lysis buffer used in the method may not include DNase or a protease and the method suitably may not include steps of adding an exogenous DNase or protease to the single tube. The differential lysis buffer used in the method suitably may be compatible with anticoagulants selected from the group consisting of EDTA, citrate, citrate dextrose (ACD) sodium polyanethole sulfonate (SPS), heparan, Sodium fluoride/oxalate, and combinations thereof.

The invention described herein suitably may include a method of concentrating and identifying a microorganism from blood. The method suitably may include steps of (a) providing a blood sample known to contain or that may contain microorganism; (b) mixing the blood sample with a differential lysis buffer comprising a buffering substance, a nonionic surfactant, and a salt, wherein the blood sample mixed with the differential lysis buffer has a pH about 7.0 to 8.0 and the buffering substance has a useful pH buffering range of about 8.6-11.4, and wherein the mixing yields a lysate comprising lysed blood cells and unlysed microorganism; (c) concentrating the microorganism from the lysate, wherein the microorganismis concentrated in a range of 25 to 100 fold relative to a starting volume of the provided blood sample; and (d) identifying the microorganism present in the blood sample, wherein the identifying is accomplished in 4 hrs or less, 3 hrs or less, 2 hrs or less, or 1 hr or less.

The identifying suitably may include one or more of a molecular test, a phenotypic test, a proteomic test, an optical test, or a culture-based test. The identifying step of the method suitably may include steps of isolating from the microorganism one or more nucleic acids characteristic of the microorganism, and analyzing the one or more nucleic acids to identify the microorganism present in the blood sample.

The nonionic surfactant recited in the method suitably may be a polyoxyethylene (POE) ether, preferably one or more of Arlasolve 200 (aka, Poly(Oxy-1,2-Ethanediyl)), Brij O10, and nonaethylene glycol monododecyl ether (aka, Brij 35). The nonionic surfactant recited in the method suitably may be selected from the group consisting of Triton X-114, NP-40, Arlasolve 200, Brij O10 (aka, Brij 96/97), octyl β-D-glucopyranoside, a saponin, nonaethylene glycol monododecyl ether (aka, Brij 35), and combinations thereof.

The buffering substance recited in the method suitably may be selected from the group consisting of CABS, CAPS, CAPSO, CHES, and combinations thereof. The buffering substance recited in the method suitably may be CAPS, wherein CAPS has a pH buffering range of about 9.7-11.1 and a pKa at 25° C. of about 10.4.

The concentration of detergent (e.g., in a range of 0.1% to 0.5%) and pH (e.g., in a range of 7-11) suitably may be adjusted to minimize pellet volume while maximizing differential lysis of blood cells in the sample. Suitably the pellet volume may be less than or equal to −500 μL, less than or equal to −400 μL, less than or equal to −300 μL, less than or equal to −200 μL, or less than or equal to −100 μL. Suitably up to 50%, 60%, 70%, 80%, 90%, 95%, 97%, 98%, 99% on non-microbial cells in the sample may be lysed within 2-5 minutes of combining the sample with the differential lysis buffer.

The salt recited in the method suitably may be sodium chloride.

The method suitably may not include a blood culture step prior to the concentrating and/or a DNase step to digest genomic DNA in the lysate.

Steps (a)-(c) of the method suitably may be completed in a time range of about 10 to 20 minutes. The isolating and analyzing steps of the method suitably may be completed in a time range of about 5 to 75 minutes. The time to yield the lysate suitably may be a range of about 2 to 10 minutes, preferably about 3-5 minutes. Yielding the lysate suitably may include no additional steps besides the combining (i.e., besides combining the blood sample with the differential lysis buffer and incubating the blood/buffer mixture for a period of time sufficient to lyse the blood cells in the sample (about 2 to 10 minutes, preferably about 3-5 minutes)).

What is described is:
A1. A method of isolating and identifying a microorganism, comprising:
(a) providing a blood sample known to contain or that may contain microorganisms;
(b) mixing the blood sample with a differential lysis buffer having a pH to yield a lysate, wherein the lysate comprises lysed blood cells and unlysed microorganism;
(c) separating the microorganisms from the lysate;
(d) adding the microorganisms to an assay device that includes one or more reagents needed for identifying the microorganisms; and
(e) identifying the microorganisms present in the blood sample, wherein the identifying includes steps of isolating from the microorganisms one or more nucleic acids characteristic of the microorganisms, and analyzing the one or more nucleic acids to identify the microorganisms present in the blood sample.
A2. The method of clause A1, wherein the microorganisms are one or more of bacteria or yeast associated with a bloodborne infection
A3. The method of clauses A1 and/or A2, wherein the method further includes initially identifying one or more symptoms of sepsis, septic infection, septic shock, septicemia, or the like in the patient to determine that the patient has a bloodborne infection.
A4. The method of one or more of clauses A1-A3, wherein the differential lysis buffer comprises a buffering agent, a nonionic surfactant, and a pH range of about 10-11 prior to mixing the blood sample with the differential lysis buffer and a pH of about 7.0 to 8.0 after mixing the blood sample with the differential lysis buffer.
A5. The method of one or more of clauses A1-A4, wherein the nonionic surfactant is a polyoxyethylene (POE) ether.
A6. The method of one or more of clauses A1-A5, wherein the nonionic surfactant is selected from the group consisting of Triton X-114, NP-40, Arlasolve 200, Brij O10 (aka, Brij 96/97), octyl β-D-glucopyranoside, a saponin, nonaethylene glycol monododecyl ether (C12E9, polidocenol), and combinations thereof.
A7. The method of one or more of clauses A1-A6, wherein separating the microorganisms from the lysate includes a centrifugation step, and the separating further comprises recovering a pellet fraction comprising the microorganisms from a supernatant fraction comprising a lysed blood fraction.
A8. The method of one or more of clauses A1-A7, further comprising:
disposing the blood sample mixed with the differential lysis buffer into a centrifugal concentrator, wherein the centrifugal concentrator comprises:
a chamber having an opening at a first end and a seal portion at a second end, wherein the seal portion is configured to seal a second opening at the second end of the chamber; and
a plunger movably disposed at least partially inside the chamber, wherein the plunger is configured to be actuated to open the seal;
centrifuging the centrifugal concentrator to pellet the microorganisms from the blood sample disposed within the chamber; and
depressing the plunger to open the seal portion to express the pellet from the opening at the second end of the chamber.
A9. The method of one or more of clauses A1-A8, wherein depressing the plunger to open the seal portion to express the pellet comprises depressing the plunger into sealing engagement with a portion of the body, and expelling the pellet from the second end under pressure by opening the seal.
A10. The method of one or more of clauses A1-A9, further comprising expressing the pellet from the the second end of the centrifugal concentrator into a vial or a self-contained assay device.
A11. The method of one or more of clauses A1-A10, wherein the centrifugal concentrator and the vial are each configured for coupling the second end of the centrifugal concentrator to the vial.
A12. The method of one or more of clauses A1-A11, wherein the vial is configured for delivering the pellet into the self-contained molecular analysis device.
A13. The method of one or more of clauses A1-A12, wherein the vial is configured for delivering the pellet into the self-contained molecular analysis device without decoupling the vial from the second end of the centrifugal concentrator.
A14. The method of one or more of clauses A1-A13, wherein the opening at the first end of the centrifugal concentrator comprises a septum, a similarly functional structure, or the like configured for aseptically loading the sample mixed with the differential lysis buffer into the centrifugal concentrator.
A15. The method of one or more of clauses A1-A14, wherein the centrifugal concentrator does not include a density cushion or a physical separator.
A16. The method of one or more of clauses A1-A15, wherein the method does not include one or more of mixing the blood sample and the differential lysis buffer in a first container and then transferring the lysate to the centrifugal concentrator, including components other than the blood sample and the differential lysis buffer in the centrifugal concentrator, opening the centrifugal concentrator to decant a supernatant fraction after centrifugation, pretreating the blood sample, a blood culture step, a step of subculturing the blood sample to identify the microorganism present in the blood sample, or a DNase step to digest genomic DNA in the lysate.
A17. The method of one or more of clauses A1-A16, wherein the method does not include one or more of pretreating the blood sample, a blood culture step, a step of subculturing the blood sample to identify the microorganisms present in the blood sample, or a DNase step to digest genomic DNA in the lysate.
A18. The method of one or more of clauses A1-A17, wherein steps (a)-(c) are completed in a time range of about 10 to 20 minutes.
A19. The method of one or more of clauses A1-A18, wherein steps (d) and (e) are completed in a time range of about 15 to 75 minutes.
A20. The method of one or more of clauses A1-A19, wherein steps (a)-(e) are completed in a time range of about 25 to 95 minutes.
A21. The method of one or more of clauses A1-A20, wherein the microorganisms are separated from the lysate by a filter.
A22. The method of one or more of clauses A1-A21, further comprising adding the filter to a self-contained assay device.
A23. The method of one or more of clauses A1-A22, wherein the differential lysis buffer comprises a buffering substance having a pH buffering range, and wherein the pH of the differential lysis buffer mixed with the blood sample is outside the pH buffering range of the buffering substance.
A23.1 The method of one or more of clauses A1-A23, wherein the buffering substance is selected from the group consisting of CABS, CAPS, CAPS, CHES, and combinations thereof.
A23.2 The method of one or more of clauses A1-A23.1, wherein the buffering substance is CAPS.
A24. The method of one or more of clauses A1-A23.2, wherein the pH of the differential lysis buffer mixed with the blood sample is below the pH buffering range of the buffering substance.
A25. The method of one or more of clauses A1-A24, wherein the pH of the differential lysis buffer mixed with the blood sample is about 1.5 to 2.5 pH units below the pH buffering range of the buffering substance.
A26. The method of one or more of clauses A1-A25, wherein the blood sample mixed with the differential lysis buffer has a pH about 7.0 to 8.0 and the buffering substance has a useful pH buffering range of about 8.6-11.4 and a pKa at 25° C. in a range of about 9.5 to about 10.7.
A27. The method of one or more of clauses A1-A26, wherein the identifying further comprises amplifying one or more nucleic acids and then detecting the one or more amplified nucleic acids.
A28. The method of one or more of clauses A1-A27, wherein the detecting the one or more amplified nucleic acids includes a nucleic acid melting step.
A29. The method of one or more of clauses A1-A28, further comprising performing a first-stage multiplex amplification on the one or more nucleic acids to yield a first-stage amplification product, diluting the first-stage amplification product, dividing the diluted first-stage amplification product among a set of second-stage amplification wells, each second-stage amplification well having a set of amplification primers configured for further amplifying a specific nucleic acid that may be present in the sample, performing a second-stage amplification in the second-stage amplification wells, and performing a post-amplification nucleic acid melt and melting-curve analysis to identify the microorganisms present in the blood sample.
A30. The method of one or more of clauses A1-A29, wherein analyzing includes a nucleic acid sequencing step to generate a sequencing data that includes sequence information derived from the one or more nucleic acids sufficient to identify the microorganisms present in the blood sample.
A31. The method of one or more of clauses A1-A30, wherein the nucleic acid sequencing step includes a massively parallel or next generation sequencing technique.
A32. The method of one or more of clauses A1-A31, wherein the steps of mixing the blood sample with the differential lysis buffer, yielding the lysate, and separating the microorganisms from the lysate are accomplished in a single tube.
A33. The method of one or more of clauses A1-A32, wherein the differential lysis buffer is a single buffer provided in the single tube.
A34. The method of one or more of clauses A1-A33, wherein the differential lysis buffer does not include DNase or a protease and the method does not include steps of adding an exogenous DNase or protease to the single tube.
A35. The method of one or more of clauses A1-A34, wherein the differential lysis buffer is compatible with standard anticoagulants such as, but not limited to, those selected from the group consisting of EDTA, citrate, sodium polyanethole sulfonate (SPS), heparan, Sodium fluoride/oxalate, and combinations thereof.
B1. A method of isolating and identifying a microorganism, comprising:
(a) providing a blood sample known to contain or that may contain microorganisms;
(b) mixing the blood sample with a differential lysis buffer comprising a buffering substance and a nonionic surfactant, and wherein the blood sample mixed with the differential lysis buffer has a pH about 7.0 to 8.0 and the buffering substance has a useful pH buffering range of about 8.6-11.4, and wherein the mixing yields a lysate comprising lysed blood cells and unlysed microorganism;
(c) separating the microorganisms from the lysate;
(d) adding the microorganisms to a self-contained assay device configured to perform an assay that includes amplification of one or more nucleic acids characteristic of the microorganisms and an analysis of the amplified one or more nucleic acids to identify the microorganisms present in the blood sample.
B2. The method of clause B1, wherein prior to amplification the assay further comprises lysis of the microorganisms and recovery of nucleic acids from the microorganisms, wherein the recovered nucleic acids are subjected to amplification for identification of the microorganisms present in the blood sample.
B3. The method of one or more of clauses B1 or B2, wherein the nonionic surfactant is a polyoxyethylene (POE) ether.
B4. The method of one or more of clauses B1-B3, wherein the nonionic surfactant is selected from the group consisting of Triton X-114, NP-40, Arlasolve 200, Brij O10 (aka, Brij 96/97), octyl β-D-glucopyranoside, a saponin, nonaethylene glycol monododecyl ether (C12E9, polidocenol), and combinations thereof.
B5. The method of one or more of clauses B1-B4, wherein the buffering substance is selected from the group consisting of CABS, CAPS, CAPSO, CHES, and combinations thereof.
B6. The method of one or more of clauses B1-B5, wherein the buffering substance is CAPS, and wherein CAPS has a pH buffering range of about 9.7-11.1 and a pKa at 25° C. of about 10.4.
B7. The method of one or more of clauses B1-B6, further comprising: combining the blood sample with the differential lysis buffer in a centrifugal concentrator, wherein the centrifugal concentrator comprises:
a chamber having an opening at a first end and a seal portion at a second end, wherein the seal portion is configured to seal a second opening at the second end of the chamber; and
a plunger movably disposed at least partially inside the chamber, wherein the plunger is configured to be actuated to open the seal;
combining the blood sample with the differential lysis buffer for a first time to yield the lysate;
centrifuging the centrifugal concentrator to pellet the microorganisms from the blood sample disposed within the chamber; and
depressing the plunger to open the seal portion to express the pellet from the opening at the second end of the chamber.
B8. The method of one or more of clauses B1-B7, wherein depressing the plunger to open the seal portion to express the pellet comprises depressing the plunger into sealing engagement with a portion of the body, and expelling the pellet from the second end under pressure by opening the seal.
B9. The method of one or more of clauses B1-B8, further comprising expressing the pellet into a cannulated vial comprising a vial body having an interior volume optionally having a sample buffer disposed therein and a cannula extending away from a bottom surface of the vial body;
the cannula having a first end and a second end, the first end of the cannula adjacent to the bottom surface of the vial body, wherein the cannula does not extend into the vial body,
the vial body further comprising a filter located near the bottom surface of the vial body configured to filter a fluid prior to entering the cannula, wherein the filter has a pore size of sufficient diameter to allow fungal, viral, protozoans, and/or bacterial organisms to pass therethrough into the cannula, but small enough to capture larger particulate matter.
B10. The method of one or more of clauses B1-B9, further comprising placing the second end of the cannula into a first port of a self-contained assay device, wherein the first port of the self-contained assay device is provided under vacuum so as to draw a volume of fluid out of the vial body through the cannula into the self-contained assay device.
B11. The method of one or more of clauses B1-B10, wherein the self-contained assay device further comprises:
a cell lysis zone fluidly connected to the first port, the cell lysis zone configured for lysing the microorganisms;
a nucleic acid preparation zone fluidly connected to the cell lysis zone, the nucleic acid preparation zone configured for purifying nucleic acids from the microorganisms;
a first-stage reaction zone fluidly connected to the nucleic acid preparation zone, the first-stage reaction zone comprising a first-stage reaction chamber configured for first-stage amplification of nucleic acids purified from the microorganisms; and
a second-stage reaction zone fluidly connected to the first-stage reaction zone, the second-stage reaction zone comprising a plurality of second-stage reaction chambers, each second-stage reaction chamber comprising a pair of primers configured for further amplification an organism-specific nucleic acid purified from the microorganisms, the second-stage reaction zone configured for contemporaneous thermal cycling of all of the plurality of second-stage reaction chambers and for performing a post-amplification nucleic acid melt and melting-curve analysis to identify the microorganisms present in the blood sample.
B12. The method of one or more of clauses B1-B11, wherein the centrifugal concentrator and the vial body of the cannulated vial are configured for engaging with one another to couple the centrifugal concentrator to the cannulated vial, and wherein the vial body of the cannulated vial is configured to surround the second end, such that the vial body of the cannulated vial is configured to collect the pellet expressed from the second end of the chamber.
B13. The method of one or more of clauses B1-B12, wherein the second end of the centrifugal concentrator includes a first engaging portion and the vial body of the cannulated vial includes a second complementary engaging portion for fixedly coupling the centrifugal concentrator to the cannulated vial.
B14. The method of one or more of clauses B1-B13, wherein the first and second engaging portions include threads for threadably coupling the centrifugal concentrator to the cannulated vial.
B15. The method of one or more of clauses B1-B14, further comprising leaving the centrifugal concentrator and the cannulated vial in engagement with one another for drawing the volume of fluid out of the vial body through the cannula into the self-contained assay device, removing the cannula of the cannulated vial from the first port of the self-contained assay device, and disposing of the centrifugal concentrator and the cannulated vial.
B16. The method of one or more of clauses B1-B15, wherein the opening at the first end of the centrifugal concentrator comprises a septum or the like configured for aseptically loading the sample mixed with the differential lysis buffer into the centrifugal concentrator.
B17. The method of one or more of clauses B1-B16, wherein the centrifugal concentrator does not include a density cushion.
B18. The method of one or more of clauses B1-B17, wherein the method does not include one or more of pretreating the blood sample, a blood culture step, a step of subculturing the blood sample to identify the microorganisms present in the blood sample, or a DNase step to digest genomic DNA in the lysate.
B19. The method of one or more of clauses B1-B18, wherein steps (a)-(c) are completed in a time range of about 10 to 20 minutes.
B20. The method of one or more of clauses B1-B19, wherein steps (d) and (e) are completed in a time range of about 15 to 75 minutes.
B21. The method of one or more of clauses B1-B20, wherein steps (a)-(e) are completed in a time range of about 25 to 95 minutes.
B22. The method of one or more of clauses B1-B21, wherein the first time to yield the lysate is in a range of about 2 to 10 minutes, preferably about 5 minutes.
B23. The method of one or more of clauses B1-B22, wherein yielding the lysate includes no additional steps besides the combining.
C1. A composition, comprising
a blood sample known to contain or that may contain a microorganism; and
a differential lysis buffer that is combined with the blood sample, the differential lysis buffer comprising an aqueous medium, a buffering substance, and a nonionic surfactant,
wherein the composition has a pH of about 7.0 to 8.0 with the buffering substance having a useful pH buffering range of about 8.6-11.4 and having a pKa at 25° C. in a range of about 9.5 to about 10.7.
C2. The composition of clause C1, wherein the nonionic surfactant is a polyoxyethylene (POE) ether.
C3. The composition of one or more of clauses C1 or C2, wherein the nonionic surfactant is selected from the group consisting of Triton X-114, NP-40, Arlasolve 200, Brij O10 (aka, Brij 96/97), octyl β-D-glucopyranoside, a saponin, nonaethylene glycol monododecyl ether (C12E9, polidocenol), and combinations thereof.
C4. The composition of one or more of clauses C1-C3, wherein the buffering substance is selected from the group consisting of CABS, CAPS, CAPSO, CHES, and combinations thereof.
C5. The composition of one or more of clauses C1-C4, wherein the buffering substance is CAPS having a pH buffering range of about 9.7-11.1 and a pKa at 25° C. of about 10.4.
C6. The composition of one or more of clauses C1-C5, wherein the buffering substance is substantially positively charged at the pH of about 7.0 to 8.0.
C7. The composition of one or more of clauses C1-C6, wherein composition does not include DNase.
C8. The composition of one or more of clauses C1-C7, consisting essentially of
a blood sample known to contain or that may contain microorganisms; and
a differential lysis buffer comprising a buffering substance and a nonionic surfactant,
wherein the composition has a pH of about 7.0 to 8.0 with the buffering substance being CAPS having a useful pH buffering range of about 9.7-11.1 and a pKa at 25° C. of about 10.4.
D1. A system, comprising
a composition comprising:
a blood sample known to contain or that may contain microorganisms; and
a differential lysis buffer comprising a buffering substance and a nonionic surfactant, wherein the composition has a pH about 7.0 to 8.0 with the buffering substance having a useful pH buffering range of about 8.6-11.4 and a pKa at 25° C. in a range of about 9.5 to about 10.7,
wherein the composition comprises a lysate that includes lysed blood cells and, if present, unlysed microorganism;
a centrifugal concentrator configured for pelleting the unlysed microorganism in the lysate, the centrifugal concentrator comprising:
   a chamber having an opening at a first end and a seal portion at a second end, wherein the seal portion is configured to seal a second opening at the second end of the chamber; and
   a plunger movably disposed at least partially inside the chamber, wherein the plunger is configured to be actuated to open the seal; and
a cannulated vial configured to be coupled to the second end of the centrifugal concentrator to receive a microorganism pellet from the centrifugal concentrator, the cannulated vial comprising
   a vial body having an interior volume optionally containing a sample buffer therein and a cannula extending away from a bottom surface of the vial body, the cannula having a first end and a second end, the first end of the cannula adjacent to the bottom surface of the vial body, wherein the cannula does not extend into the vial body; and
   the vial body further comprising a filter located near the bottom surface of the vial body configured to filter a fluid prior to entering the cannula, wherein the filter has a pore size of sufficient diameter to allow fungal, viral, protozoans, and/or bacterial organisms to pass therethrough into the cannula, but small enough to capture larger particulate matter.
D2. The system of clause D1, wherein the centrifugal concentrator does not include a density cushion.
D3. The system of one or more of clauses D1 and D2, further comprising a self-contained assay device having a first port configured to receive the second end of the cannula for introduction of a sample in the self-contained assay device, wherein the first port of the self-contained assay device is provided under vacuum so as to draw a volume of the sample out of the vial body through the cannula into the self-contained assay device.
D4. The system of one or more of clauses D1-D3, wherein the self-contained assay device further comprises:
a cell lysis zone fluidly connected to the first port, the cell lysis zone configured for lysing the microorganisms;
a nucleic acid preparation zone fluidly connected to the cell lysis zone, the nucleic acid preparation zone configured for purifying nucleic acids from the microorganisms;
a first-stage reaction zone fluidly connected to the nucleic acid preparation zone, the first-stage reaction zone comprising a first-stage reaction chamber configured for first-stage amplification of nucleic acids purified from the microorganisms; and
a second-stage reaction zone fluidly connected to the first-stage reaction zone, the second-stage reaction zone comprising a plurality of second-stage reaction chambers, each second-stage reaction chamber comprising a pair of primers configured for further amplification an organism-specific nucleic acid purified from the microorganisms, the second-stage reaction zone configured for contemporaneous thermal cycling of all of the plurality of second-stage reaction chambers and for execution of a post-amplification nucleic acid melt and melting-curve analysis to identify the microorganisms present in the blood sample.
D5. The system of one or more of clauses D1-D4, wherein the centrifugal concentrator and the vial body of the cannulated vial are configured for engaging with one another to couple the centrifugal concentrator to the cannulated vial, and wherein the vial body of the cannulated vial is configured to surround the second end, such that the vial body of the cannulated vial is configured to collect the pellet expressed from the second end of the chamber.
D6. The system of one or more of clauses D1-D5, wherein the second end of the centrifugal concentrator includes a first engagement portion and the vial body of the cannulated vial includes a complementary second engagement portion for fixedly coupling the centrifugal concentrator to the cannulated vial.
D7. The system of one or more of clauses D1-D6, wherein the first and second engagements portions include threads for threadably coupling the centrifugal concentrator to the cannulated vial.
D8. The system of one or more of clauses D1-D7, wherein the opening at the first end of the centrifugal concentrator comprises a septum or the like configured for aseptically loading the sample mixed with the differential lysis buffer into the centrifugal concentrator.
D9. The system of one or more of clauses D1-D8, wherein the centrifugal concentrator does not include a density cushion.
D10. The system of one or more of clauses D1-D9, wherein the nonionic surfactant of the differential lysis buffer is a polyoxyethylene (POE) ether.
D11. The system of one or more of clauses D1-D10, wherein the nonionic surfactant of the differential lysis buffer is selected from the group consisting of Triton X-114, NP-40, Arlasolve 200, Brij O10 (aka, Brij 96/97), octyl β-D-glucopyranoside, a saponin, nonaethylene glycol monododecyl ether (C12E9, polidocenol), and combinations thereof.
D12. The system of one or more of clauses D1-D11, wherein the buffering substance of the differential lysis buffer is selected from the group consisting of CABS, CAPS, CAPSO, CHES, and combinations thereof.
D13. The system of one or more of clauses D1-D12, wherein the buffering substance of the differential lysis buffer is CAPS, and wherein CAPS has a useful pH buffering range of about 9.7-11.1 and a pKa at 25° C. of about 10.4.
D14. The system of one or more of clauses D1-D13, wherein the buffering substance of the differential lysis buffer is substantially positively charged at the pH of about 7.0 to 8.0.
D15. The system of one or more of clauses D1-D14, wherein the differential lysis buffer does not include DNase.
E1. A method of isolating and identifying a microorganism, comprising:
(a) providing a blood sample known to contain or that may contain microorganisms;
(b) mixing the blood sample with a differential lysis buffer having a pH to yield a lysate, wherein the lysate comprises lysed blood cells and unlysed microorganism;
(c) disposing the blood sample mixed with the differential lysis buffer into a centrifugal concentrator, wherein the centrifugal concentrator comprises:
   a chamber having an opening at a first end and a seal portion at a second end, wherein the seal portion is configured to seal a second opening at the second end of the chamber; and
   a plunger movably disposed at least partially inside the chamber, wherein the plunger is configured to be actuated to open the seal;
(d) centrifuging the centrifugal concentrator to pellet the microorganisms from the blood sample disposed within the chamber;
(e) adding the microorganisms to a self-contained assay device that includes one or more reagents needed for identifying the microorganisms, wherein adding the microorganisms to the self-contained assay device includes depressing the plunger to open the seal portion to express the pellet from the opening at the second end of the chamber; and
(f) identifying the microorganisms present in the blood sample, wherein the identifying includes steps of isolating from the microorganisms one or more nucleic acids characteristic of the microorganisms, performing a nucleic acid amplification, and performing a post-amplification nucleic acid melt and melting-curve analysis to identify the microorganisms present in the blood sample.
E2. The method of clause E1, wherein the self-contained assay device further comprises:
a first port provided under vacuum so as to draw a volume of the pellet into the self-contained assay device;
a cell lysis zone fluidly connected to the first port, the cell lysis zone configured for lysing the microorganisms;
a nucleic acid preparation zone fluidly connected to the cell lysis zone, the nucleic acid preparation zone configured for purifying nucleic acids from the microorganisms;
a first-stage reaction zone fluidly connected to the nucleic acid preparation zone, the first-stage reaction zone comprising a first-stage reaction chamber configured for performing a first-stage multiplex amplification on the one or more nucleic acids;
a second-stage reaction zone fluidly connected to the first-stage reaction zone, the second-stage reaction zone comprising a plurality of second-stage reaction chambers, each second-stage reaction chamber comprising a pair of primers configured for further amplification of a specific nucleic acid purified from one of the microorganisms, the second-stage reaction zone configured for contemporaneous thermal cycling of all of the plurality of second-stage reaction chambers, and
the method further comprising performing the first-stage multiplex amplification in the first-stage reaction zone to yield a first-stage amplification product, diluting the first-stage amplification product, dividing the diluted first-stage amplification product among the plurality of second-stage reaction chambers, performing a second-stage amplification in the second-stage amplification chambers, and performing the post-amplification nucleic acid melt and melting-curve analysis after the second-stage amplification to identify the microorganisms present in the blood sample.
E3. The method of one or more of clauses E1 and E2, wherein depressing the plunger to open the seal portion to express the pellet comprises depressing the plunger into sealing engagement with a portion of the body, and expelling the pellet from the second end under pressure by opening the seal.
E4. The method of one or more of clauses E1-E3, further comprising expressing the pellet into a cannulated vial having a sample buffer therein, the cannulated vial comprising a vial body having an interior volume and a cannula extending away from a bottom surface of the vial body;
the cannula having a first end and a second end, the first end of the cannula adjacent to the bottom surface of the vial body, wherein the cannula does not extend into the vial body,
the vial body further comprising a filter located near the bottom surface of the vial body configured to filter a fluid prior to entering the cannula, wherein the filter has a pore size of sufficient diameter to allow fungal, viral, protozoans, and/or bacterial organisms to pass therethrough into the cannula, but small enough to capture larger particulate matter.
E5. The method of one or more of clauses E1-E4, further comprising placing the second end of the cannula into a first port of the self-contained assay device, wherein the first port of the self-contained assay device is provided under vacuum so as to draw a volume of fluid out of the vial body through the cannula into the self-contained assay device.
E6. The method of one or more of clauses E1-E5, wherein steps (b)-(d) are performed in a single tube.
E7. The method of one or more of clauses E1-E6, wherein the differential lysis buffer is a single buffer provided in the single tube.
E8. The method of one or more of clauses E1-E7, wherein the differential lysis buffer does not include DNase or a protease and the method does not include steps of adding an exogenous DNase or protease to the to the single tube.
E9. The method of one or more of clauses E1-E8, wherein the method does not include one or more of pretreating the blood sample, a blood culture step, a step of subculturing the blood sample to identify the microorganisms present in the blood sample, or a DNase step to digest genomic DNA in the lysate.
E10. The method of one or more of clauses E1-E9, wherein steps (a)-(d) are completed in a time range of about 10 to 20 minutes.
E11. The method of one or more of clauses E1-E10, wherein steps (e) and (f) are completed in a time range of about 15 to 75 minutes.
E12. The method of one or more of clauses E1-E11, wherein steps (a)-(f) are completed in a time range of about 25 to 95 minutes.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description that follows, and in part will be obvious from the description, or may be learned by the practice of the invention. The features and advantages may be realized and obtained by means of the instruments and combinations particularly pointed out in the appended claims. These and other features will become more fully apparent from the following description and appended claims, or may be learned by the practice of the invention as set forth hereinafter.

### BRIEF DESCRIPTION OF THE FIGURES

FIG. 1 shows a flexible pouch useful for self-contained PCR.

FIG. 2 is an exploded perspective view of an instrument for use with the pouch of FIG. 1, including the pouch of FIG. 1.

FIG. 3 shows the pouch of FIG. 1 along with the bladder components of FIG. 2.

FIG. 4 shows a motor used in one illustrative embodiment of the instrument of FIG. 2.

FIG. 5 is a schematic illustration of one embodiment of a differential lysis and centrifugation method with systems and apparatuses described herein.

FIG. 6A is an isometric view of a centrifugal concentrator, according to one embodiment of the present invention.

FIG. 6B is a side view of the centrifugal concentrator of FIG. 6A.

FIG. 6C shows the same view of the centrifugal concentrator as in FIG. 6B with the cap removed.

FIG. 6D is another isometric view of a centrifugal concentrator.

FIG. 6E is a detailed view of one end of the centrifugal concentrator.

FIG. 6F is a cut-away view of the end of the centrifugal concentrator shown in FIG. 6E.

FIG. 6G is an isometric view of the plunger of the centrifugal concentrator of FIGS. 6A-6F.

FIG. 7 is an example of a workflow using the differential lysis buffer.

FIG. 8 is a bar graph comparing the differential lysis buffer to several other protocols.

FIG. 9 is a bar graph illustrating cell recovery with the differential lysis buffer.

FIG. 10 illustrates the effect of removing human genomic DNA with an illustrative differential lysis and centrifugation procedure.

FIG. 11 is data showing that the differential lysis buffer can selectively lyse eukaryotic host cells while leaving microorganism cells intact.

FIG. 12 illustrates a workflow for recovery and detection efficiency of microorganisms from whole blood.

FIG. 13 illustrates the average recovery of microorganisms in the study workflow illustrated in FIG. 12.

FIG. 14 illustrates the average inoculum and recovery of microorganisms in the study workflow illustrated in FIG. 12.

FIG. 15 A-C illustrates a flow through method for animal cells lysis, culturing, and concentration of microorganisms.

FIG. 16 illustrates a filtration method for isolation and concentration of microorganisms.

FIG. 17 A-C schematically illustrate different filtration structures that can be used to separate cells by size.

FIG. 18 schematically illustrates different types of pillar filters (18A) polygonal, (18B) U-shaped, and (18C) butterfly-shaped micropillar geometries.

FIG. 19 schematically illustrates separation of large and small cells in a structure with an array of micopillars and cross-flows of buffer and cell suspension.

FIG. 20 schematically illustrates the concentration large and small cells by migration along an oval-shaped filter unit.

FIG. 21 is an absorbance vs. incubation time graph illustrating lysis of blood samples over time with various differential lysis buffer formulations.

FIG. 22. is a bar graph illustrating the increase in organism concentration for several types of organisms and blood anticoagulants.

### DETAILED DESCRIPTION

Example embodiments are described below with reference to the accompanying drawings. Many different forms and embodiments are possible without deviating from the spirit and teachings of this disclosure and so the disclosure should not be construed as limited to the example embodiments set forth herein. Rather, these example embodiments are provided so that this disclosure will be thorough and complete, and will convey the scope of the disclosure to those skilled in the art. In the drawings, the sizes and relative sizes of layers and regions may be exaggerated for clarity. Like reference numbers refer to like elements throughout the description.

Unless defined otherwise, all terms (including technical and scientific terms) used herein have the same meaning as commonly understood by one of ordinary skill in the art to which the present disclosure pertains. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. While a number of methods and materials similar or equivalent to those described herein can be used in the practice of the present disclosure, only certain exemplary materials and methods are described herein.

All publications, patent applications, patents or other references mentioned herein are incorporated by reference for in their entirety. In case of a conflict in terminology, the present specification is controlling.

Various aspects of the present disclosure, including devices, systems, methods, etc., may be illustrated with reference to one or more exemplary implementations. As used herein, the terms “exemplary” and “illustrative” mean “serving as an example, instance, or illustration,” and should not necessarily be construed as preferred or advantageous over other implementations disclosed herein. In addition, reference to an “implementation” or “embodiment” of the present disclosure or invention includes a specific reference to one or more embodiments thereof, and vice versa, and is intended to provide illustrative examples without limiting the scope of the invention, which is indicated by the appended claims rather than by the following description.

It will be noted that, as used in this specification and the appended claims, the singular forms “a,” “an,” and “the” include plural referents unless the content clearly dictates otherwise. Thus, for example, reference to “a tile” includes one, two, or more tiles. Similarly, reference to a plurality of referents should be interpreted as comprising a single referent and/or a plurality of referents unless the content and/or context clearly dictate otherwise. Thus, reference to “tiles” does not necessarily require a plurality of such tiles. Instead, it will be appreciated that independent of conjugation; one or more tiles are contemplated herein.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

As used herein, directional and/or arbitrary terms, such as “top,” “bottom,” “left,” “right,” “up,” “down,” “upper,” “lower,” “inner,” “outer,” “internal,” “external,” “interior,” “exterior,” “proximal,” “distal,” “forward,” “reverse,” and the like can be used solely to indicate relative directions and/or orientations and may not be otherwise intended to limit the scope of the disclosure, including the specification, invention, and/or claims.

It will be understood that when an element is referred to as being “coupled,” “connected,” or “responsive” to, or “on,” another element, it can be directly coupled, connected, or responsive to, or on, the other element, or intervening elements may also be present. In contrast, when an element is referred to as being “directly coupled,” “directly connected,” or “directly responsive” to, or “directly on,” another element, there are no intervening elements present.

Example embodiments of the present inventive concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the present inventive concepts should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of example embodiments.

It will be understood that although the terms “first,” “second,” etc. may be used herein to describe various elements, these elements should not be limited by these terms. These terms are only used to distinguish one element from another. Thus, a “first” element could be termed a “second” element without departing from the teachings of the present embodiments.

It is also understood that various implementations described herein can be utilized in combination with any other implementation described or disclosed, without departing from the scope of the present disclosure. Therefore, products, members, elements, devices, apparatuses, systems, methods, processes, compositions, and/or kits according to certain implementations of the present disclosure can include, incorporate, or otherwise comprise properties, features, components, members, elements, steps, and/or the like described in other implementations (including systems, methods, apparatus, and/or the like) disclosed herein without departing from the scope of the present disclosure. Thus, reference to a specific feature in relation to one implementation should not be construed as being limited to applications only within that implementation.

The headings used herein are for organizational purposes only and are not meant to be used to limit the scope of the description or the claims. To facilitate understanding, like reference numerals have been used, where possible, to designate like elements common to the figures. Furthermore, where possible, like numbering of elements have been used in various figures. Furthermore, alternative configurations of a particular element may each include separate letters appended to the element number.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

As used herein, the term “microorganism” is intended to encompass organisms that are generally unicellular, which can be multiplied and handled in the laboratory, including but not limited to, Gram-positive or Gram-negative bacteria, yeasts, molds, and parasites. Non-limiting examples of Gram-negative bacteria of this invention include bacteria of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Stenotrophomonas, Brevundimonas, Ralstonia, Achromobacter, Fusobacterium, Prevotella, Branhamella, Neisseria, Burkholderia, Citrobacter, Hafnia, Edwardsiella, Aeromonas, Moraxella, Brucella, Pasteurella, Providencia, and Legionella. Non-limiting examples of Gram-positive bacteria of this invention include bacteria of the following genera: Enterococcus, Streptococcus, Staphylococcus, Bacillus, Paenibacillus, Lactobacillus, Listeria, Peptostreptococcus, Propionibacterium, Clostridium, Bacteroides, Gardnerella, Kocuria, Lactococcus, Leuconostoc, Micrococcus, Mycobacteria and Corynebacteria. Non-limiting examples of yeasts and molds of this invention include those of the following genera: Candida, Cryptococcus, Nocardia, Penicillium, Alternaria, Rhodotorula, Aspergillus, Fusarium, Saccharomyces and Trichosporon. Non-limiting examples of parasites of this invention include those of the following genera: Trypanosoma, Babesia, Leishmania, Plasmodium, Wucheria, Brugia, Onchocerca, and Naegleria.

In one aspect, as described in further detail herein, microorganisms from a sample or growth medium can be separated and interrogated to characterize and/or identify the microorganism present in the sample. As used herein, the term “separate” is intended to encompass any sample of microorganisms that has been removed, concentrated or otherwise set apart from its original state, or from a growth or culture medium. For example, in accordance with this invention, microorganisms may be separated away (e.g., as a separated sample) from non-microorganism or non-microorganism components that may otherwise interfere with characterization and/or identification. The term may include microorganisms that have been separated from a mixture by centrifugation, filtration, or any other separation technique known in the art. As such, a separated microorganism sample may include collection of microorganisms and/or components thereof that are more concentrated than, or otherwise set apart from, the original sample, and can range from a closely packed dense clump of microorganisms to a diffuse layer of microorganisms. Non-microorganism components that are separated away from the microorganisms may include non-microorganism cells (e.g., blood cells and/or other tissue cells) and/or any components thereof. In one aspect, the microorganisms are separated from a lysate mixture that includes lysed non-microorganism cells and substantially intact microorganism cells.

In some embodiments, separation of a sample of microorganisms from its original state, or from a growth or culture medium is incomplete. In other words, removing, concentrating, or otherwise setting the microorganisms apart from its original state does not completely separate the sample of microorganisms from other constituents of the sample or from the growth or culture medium. In some cases, a de minimis amount of debris from the sample or from the growth or culture medium is present. For example, the amount of debris or growth or culture medium present in the separated sample may be insufficient to interfere with identification or characterization of the microorganism, or further growth of the microorganism. In some embodiments, the separated sample is 99% pure of contaminating elements, but it may also be 95% pure, 90% pure, 80% pure, 70% pure, 60% pure, 50% pure, or of a minimum purity that still permits identification of the microorganism in the separated sample via a downstream identification technique.

In yet another aspect described in further detail herein, microorganisms from a sample or growth medium can be pelleted and interrogated to characterize and/or identify the microorganism present in the sample. As used herein, the term “pellet” is intended to encompass any sample of microorganisms that has been compressed or deposited into a mass of microorganisms. For example, microorganisms from a sample can be compressed or deposited into a mass at the bottom of a tube by centrifugation, or other known methods in the art. The term includes a collection of microorganisms (and/or components thereof) on the bottom and/or sides of a container following centrifugation. In accordance with this invention, microorganisms may be pelleted away (e.g., as a substantially purified microorganism pellet) from non-microorganism or non-microorganism components that may otherwise interfere with characterization and/or identification.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, mRNA, rRNA, cDNA, gDNA, ssDNA, dsDNA, or any combination thereof.

By “probe,” “primer,” or “oligonucleotide” is meant a single-stranded nucleic acid molecule of defined sequence that can base-pair to a second nucleic acid molecule that contains a complementary sequence (the “target”). The stability of the resulting hybrid depends upon the length, GC content, and the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes, primers, and oligonucleotides may be detectably-labeled, either radioactively, fluorescently, or non-radioactively, by methods well-known to those skilled in the art. dsDNA binding dyes may be used to detect dsDNA. It is understood that a “primer” is specifically configured to be extended by a polymerase, whereas a “probe” or “oligonucleotide” may or may not be so configured.

By “dsDNA binding dyes” is meant dyes that fluoresce differentially when bound to double-stranded DNA than when bound to single-stranded DNA or free in solution, usually by fluorescing more strongly. While reference is made to dsDNA binding dyes, it is understood that any suitable dye may be used herein, with some non-limiting illustrative dyes described in U.S. Pat. No. 7,387,887, herein incorporated by reference. Other signal producing substances may be used for detecting nucleic acid amplification and melting, illustratively enzymes, antibodies, etc., as are known in the art.

By “specifically hybridizes” is meant that a probe, primer, or oligonucleotide recognizes and physically interacts (that is, base-pairs) with a substantially complementary nucleic acid (for example, a sample nucleic acid) under high stringency conditions, and does not substantially base pair with other nucleic acids.

By “high stringency conditions” is meant typically to occur at about a melting temperature (Tm) minus 5° C. (i.e. 5° below the Tm of the probe). Functionally, high stringency conditions are used to identify nucleic acid sequences having at least 80% sequence identity.

By “lysis particles” is meant various particles or beads for the lysis of cells, viruses, spores, and other material that may be present in a sample. Various examples use Zirconium (“Zr”) silicate or ceramic beads, but other lysis particles are known and are within the scope of this term, including glass and sand lysis particles. The term “cell lysis component” may include lysis particles, but may also include other components, such as components for chemical lysis, as are known in the art.

While PCR is the amplification method used in the examples herein, it is understood that any amplification method that uses a primer may be suitable. Such suitable procedures include polymerase chain reaction (PCR); strand displacement amplification (SDA); nucleic acid sequence-based amplification (NASBA); cascade rolling circle amplification (CRCA), loop-mediated isothermal amplification of DNA (LAMP); isothermal and chimeric primer-initiated amplification of nucleic acids (ICAN); target based-helicase dependent amplification (HDA); transcription-mediated amplification (TMA), and the like. Therefore, when the term PCR is used, it should be understood to include other alternative amplification methods. For amplification methods without discrete cycles, reaction time may be used where measurements are made in cycles, doubling time, or crossing point (Cp), and additional reaction time may be added where additional PCR cycles are added in the embodiments described herein. It is understood that protocols may need to be adjusted accordingly.

While various examples herein reference human targets and human pathogens, these examples are illustrative only. Methods, kits, and devices described herein may be used to detect or sequence a wide variety of nucleic acid sequences from a wide variety of samples, including, human, veterinary, industrial, and environmental.

Various embodiments disclosed herein use a self-contained nucleic acid analysis pouch to assay a sample for the presence of various biological substances, illustratively antigens and nucleic acid sequences, illustratively in a single closed system. Such systems, including pouches and instruments for use with the pouches, are disclosed in more detail in U.S. Pat. Nos. 8,394,608; and 8,895,295; and 10,464,060, herein incorporated by reference. However, it is understood that such pouches are illustrative only, and the nucleic acid preparation and amplification reactions discussed herein may be performed in any of a variety of open or closed system sample vessels as are known in the art, including 96-well plates, plates of other configurations, arrays, carousels, and the like, using a variety of nucleic acid purification and amplification systems, as are known in the art. While the terms “sample well”, “amplification well”, “amplification container”, or the like are used herein, these terms are meant to encompass wells, tubes, and various other reaction containers, as are used in these amplification systems. In one embodiment, the pouch is used to assay for multiple pathogens. The pouch may include one or more blisters used as sample wells, illustratively in a closed system. Illustratively, various steps may be performed in the optionally disposable pouch, including nucleic acid preparation, primary large volume multiplex PCR, dilution of primary amplification product, and secondary PCR, culminating with optional real-time detection or post-amplification analysis such as melting-curve analysis. Further, it is understood that while the various steps may be performed in pouches of the present invention, one or more of the steps may be omitted for certain uses, and the pouch configuration may be altered accordingly. While many embodiments herein use a multiplex reaction for the first-stage amplification, it is understood that this is illustrative only, and that in some embodiments the first-stage amplification may be singleplex. In one illustrative example, the first-stage singleplex amplification targets housekeeping genes, and the second-stage amplification uses differences in housekeeping genes for identification. Thus, while various embodiments discuss first-stage multiplex amplification, it is understood that this is illustrative only.

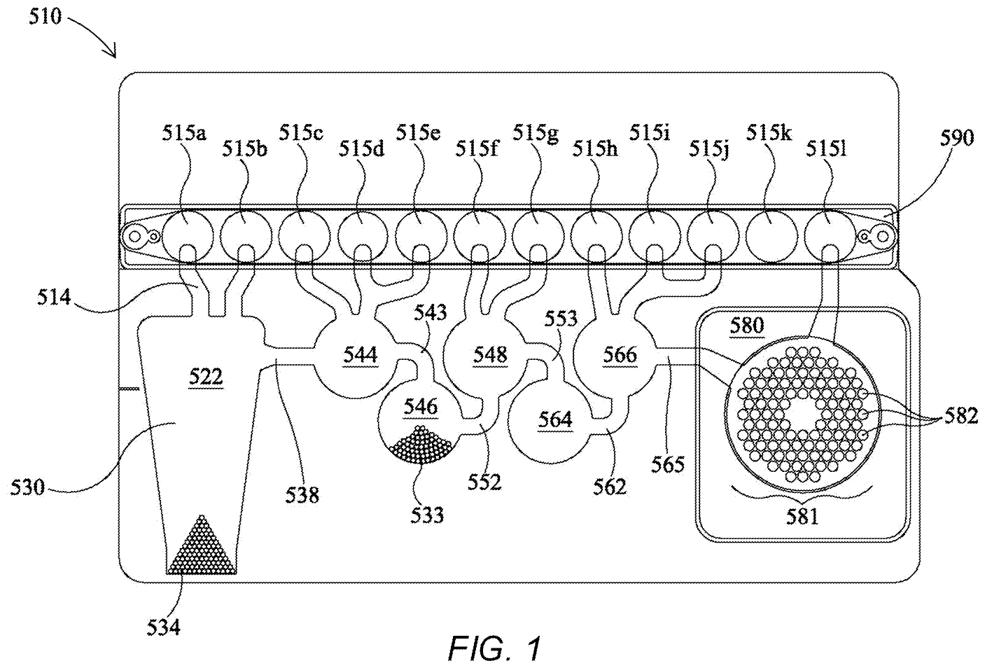

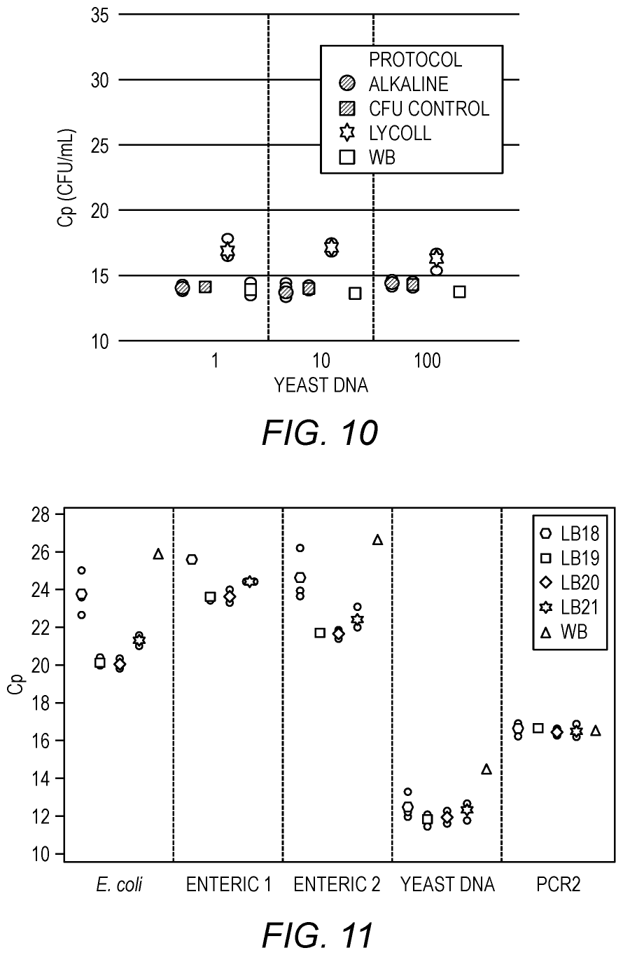

FIG. 1 shows an illustrative pouch 510 that may be used in various embodiments, or may be reconfigured for various embodiments. Pouch 510 is similar to FIG. 15 of U.S. Pat. No. 8,895,295, with like items numbered the same. Fitment 590 is provided with entry channels 515a through 515l, which also serve as reagent reservoirs or waste reservoirs. Illustratively, reagents may be freeze dried in fitment 590 and rehydrated prior to use. Blisters 522, 544, 546, 548, 564, and 566, with their respective channels 514, 538, 543, 552, 553, 562, and 565 are similar to blisters of the same number of FIG. 15 of U.S. Pat. No. 8,895,295. Second-stage reaction zone 580 of FIG. 1 is similar to that of U.S. Pat. No. 8,895,295, but the second-stage wells 582 of high density array 581 are arranged in a somewhat different pattern. The more circular pattern of high density array 581 of FIG. 1 eliminates wells in corners and may result in more uniform filling of second-stage wells 582. As shown, the high density array 581 is provided with 102 second-stage wells 582. Pouch 510 is suitable for use in the FilmArray® instrument (BioFire Diagnostics, LLC, Salt Lake City, UT). However, it is understood that the pouch embodiment is illustrative only.

While other containers may be used, illustratively, pouch 510 may be formed of two layers of a flexible plastic film or other flexible material such as polyester, polyethylene terephthalate (PET), polycarbonate, polypropylene, polymethylmethacrylate, mixtures, combinations, and layers thereof that can be made by any process known in the art, including extrusion, plasma deposition, and lamination. For instance, each layer can be composed of one or more layers of material of a single type or more than one type that are laminated together. Metal foils or plastics with aluminum lamination also may be used. Other barrier materials are known in the art that can be sealed together to form the blisters and channels. If plastic film is used, the layers may be bonded together, illustratively by heat sealing. Illustratively, the material has low nucleic acid binding and low protein binding capacity.

For embodiments employing fluorescent monitoring, plastic films that are adequately low in absorbance and auto-fluorescence at the operative wavelengths are preferred. Such material could be identified by testing different plastics, different plasticizers, and composite ratios, as well as different thicknesses of the film. For plastics with aluminum or other foil lamination, the portion of the pouch that is to be read by a fluorescence detection device can be left without the foil. For example, if fluorescence is monitored in second-stage wells 582 of the second-stage reaction zone 580 of pouch 510, then one or both layers at wells 582 would be left without the foil. In the example of PCR, film laminates composed of polyester (Mylar, DuPont, Wilmington DE) of about 0.0048 inch (0.1219 mm) thick and polypropylene films of 0.001-0.003 inch (0.025-0.076 mm) thick perform well. Illustratively, pouch 510 may be made of a clear material capable of transmitting approximately 80%-90% of incident light.

In the illustrative embodiment, the materials are moved between blisters by the application of pressure, illustratively pneumatic pressure, upon the blisters and channels. Accordingly, in embodiments employing pressure, the pouch material illustratively is flexible enough to allow the pressure to have the desired effect. The term “flexible” is herein used to describe a physical characteristic of the material of the pouch. The term “flexible” is herein defined as readily deformable by the levels of pressure used herein without cracking, breaking, crazing, or the like. For example, thin plastic sheets, such as Saran™ wrap and Ziploc® bags, as well as thin metal foil, such as aluminum foil, are flexible. However, only certain regions of the blisters and channels need be flexible, even in embodiments employing pneumatic pressure. Further, only one side of the blisters and channels need to be flexible, as long as the blisters and channels are readily deformable. Other regions of the pouch 510 may be made of a rigid material or may be reinforced with a rigid material. Thus, it is understood that when the terms “flexible pouch” or “flexible sample container” or the like are used, only portions of the pouch or sample container need be flexible.

Illustratively, a plastic film may be used for pouch 510. A sheet of metal, illustratively aluminum, or other suitable material, may be milled or otherwise cut, to create a die having a pattern of raised surfaces. When fitted into a pneumatic press (illustratively A-5302-PDS, Janesville Tool Inc., Milton WI), illustratively regulated at an operating temperature of 195° C., the pneumatic press works like a printing press, melting the sealing surfaces of plastic film only where the die contacts the film. Likewise, the plastic film(s) used for pouch 510 may be cut and welded together using a laser cutting and welding device. Various components, such as PCR primers (illustratively spotted onto the film and dried), antigen binding substrates, magnetic beads, and zirconium silicate beads may be sealed inside various blisters as the pouch 510 is formed. Reagents for sample processing can be spotted onto the film prior to sealing, either collectively or separately. In one embodiment, nucleotide tri-phosphates (NTPs) are spotted onto the film separately from polymerase and primers, essentially eliminating activity of the polymerase until the reaction may be hydrated by an aqueous sample. If the aqueous sample has been heated prior to hydration, this creates the conditions for a true hot-start PCR and reduces or eliminates the need for expensive chemical hot-start components. In another embodiment, components may be provided in powder or pill form and are placed into blisters prior to final sealing.

Pouch 510 may be used in a manner similar to that described in U.S. Pat. No. 8,895,295. In one illustrative embodiment, a 300 μl mixture comprising the sample to be tested (100 μl) and lysis buffer (200 μl) may be injected into an injection port (not shown) in fitment 590 near entry channel 515a, and the sample mixture may be drawn into entry channel 515a. Water may also be injected into a second injection port (not shown) of the fitment 590 adjacent entry channel 515l, and is distributed via a channel (not shown) provided in fitment 590, thereby hydrating up to eleven different reagents, each of which were previously provided in dry form at entry channels 515b through 515l. Illustrative methods and devices for injecting sample and hydration fluid (e.g. water or buffer) are disclosed in U.S. Patent Application No. 2014-0283945, herein incorporated by reference in its entirety, although it is understood that these methods and devices are illustrative only and other ways of introducing sample and hydration fluid into pouch 510 are within the scope of this disclosure. These reagents illustratively may include freeze-dried PCR reagents, DNA extraction reagents, wash solutions, immunoassay reagents, or other chemical entities. Illustratively, the reagents are for nucleic acid extraction, first-stage multiplex PCR, dilution of the multiplex reaction, and preparation of second-stage PCR reagents, as well as control reactions. In the embodiment shown in FIG. 1, all that need be injected is the sample solution in one injection port and water in the other injection port. After injection, the two injection ports may be sealed. For more information on various configurations of pouch 510 and fitment 590, see U.S. Pat. No. 8,895,295, already incorporated by reference.

After injection, the sample may be moved from injection channel 515a to lysis blister 522 via channel 514. Lysis blister 522 is provided with beads or particles 534, such as ceramic beads or other abrasive elements, and is configured for vortexing via impaction using rotating blades or paddles provided within the FilmArray® instrument. Bead-milling, by shaking, vortexing, sonicating, and similar treatment of the sample in the presence of lysis particles such as zirconium silicate (ZS) beads 534, is an effective method to form a lysate. It is understood that, as used herein, terms such as “lyse,” “lysing,” and “lysate” are not limited to rupturing cells, but that such terms include disruption of non-cellular particles, such as viruses. In another embodiment, a paddle beater using reciprocating or alternating paddles, such as those described in US 2019-0344269, herein incorporated by reference in its entirety, may be used for lysis in this embodiment, as well as in the other embodiments described herein.

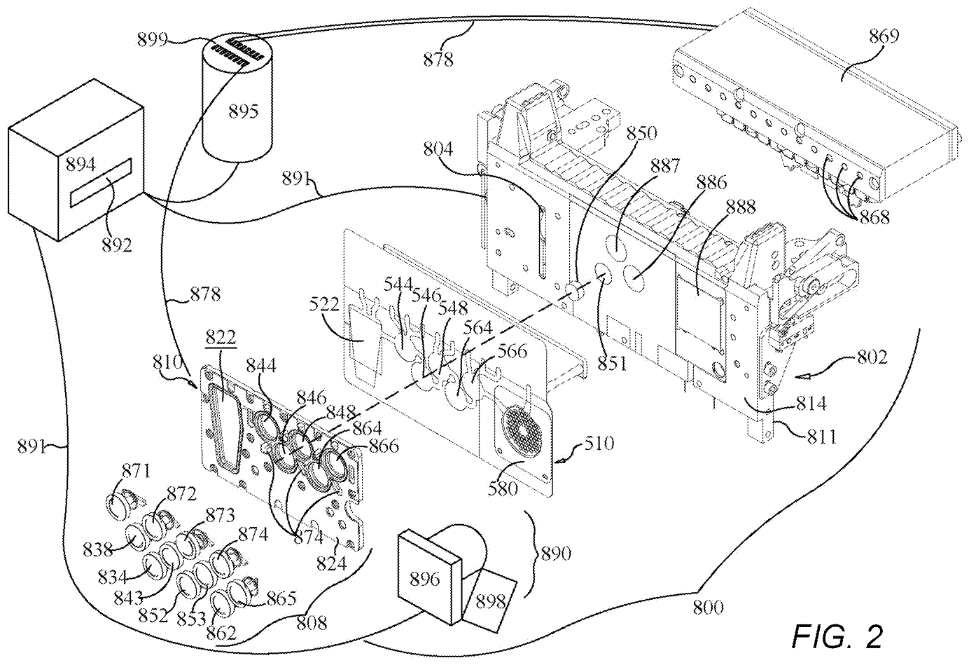

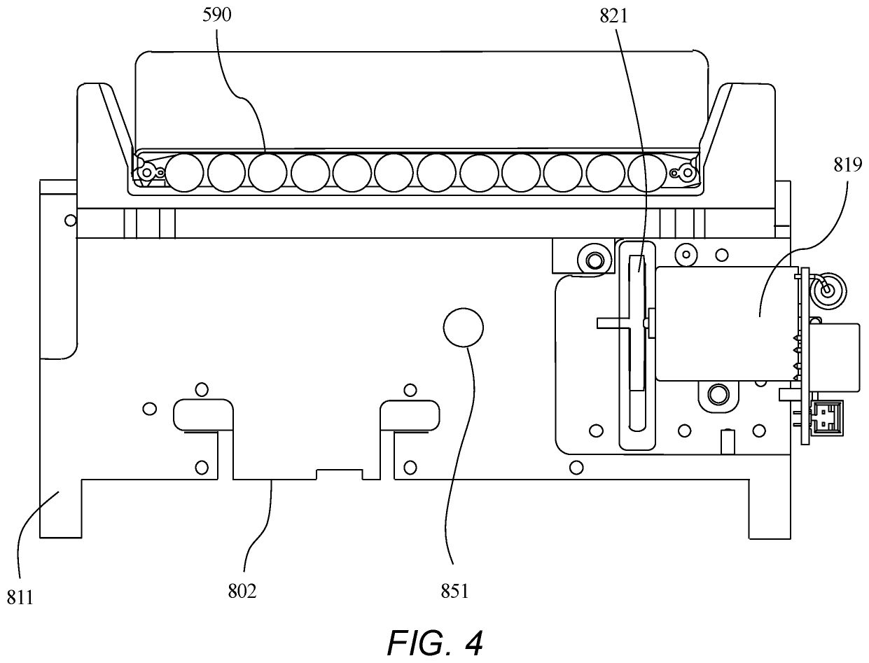

FIG. 4 shows a bead beating motor 819, comprising blades 821 that may be mounted on a first side 811 of support member 802, of instrument 800 shown in FIG. 2. Blades may extend through slot 804 to contact pouch 510. It is understood, however, that motor 819 may be mounted on other structures of instrument 800. In one illustrative embodiment, motor 819 is a Mabuchi RC-280SA-2865 DC Motor (Chiba, Japan), mounted on support member 802. In one illustrative embodiment, the motor is turned at 5,000 to 25,000 rpm, more illustratively 10,000 to 20,000 rpm, and still more illustratively approximately 15,000 to 18,000 rpm. For the Mabuchi motor, it has been found that 7.2V provides sufficient rpm for lysis. It is understood, however, that the actual speed may be somewhat slower when the blades 821 are impacting pouch 510. Other voltages and speeds may be used for lysis depending on the motor and paddles used. Optionally, controlled small volumes of air may be provided into the bladder 822 adjacent lysis blister 522. It has been found that in some embodiments, partially filling the adjacent bladder with one or more small volumes of air aids in positioning and supporting lysis blister during the lysis process. Alternatively, another structure, illustratively a rigid or compliant gasket or other retaining structure around lysis blister 522, can be used to restrain pouch 510 during lysis. It is also understood that motor 819 is illustrative only, and other devices may be used for milling, shaking, or vortexing the sample. In some embodiments, chemicals or heat may be used in addition to or instead of mechanical lysis.

Once the sample material has been adequately lysed, the sample is moved to a nucleic acid extraction zone, illustratively through channel 538, blister 544, and channel 543, to blister 546, where the sample is mixed with a nucleic acid-binding substance, such as silica-coated magnetic beads 533. Alternatively, magnetic beads 533 may be rehydrated, illustratively using fluid provided from one of the entry channel 515c-515e, and then moved through channel 543 to blister 544, and then through channel 538 to blister 522. The mixture is allowed to incubate for an appropriate length of time, illustratively approximately 10 seconds to 10 minutes. A retractable magnet located within the instrument adjacent blister 546 captures the magnetic beads 533 from the solution, forming a pellet against the interior surface of blister 546. If incubation takes place in blister 522, multiple portions of the solution may need to be moved to blister 546 for capture. The liquid is then moved out of blister 546 and back through blister 544 and into blister 522, which is now used as a waste receptacle. One or more wash buffers from one or more of injection channels 515c to 515e are provided via blister 544 and channel 543 to blister 546. Optionally, the magnet is retracted and the magnetic beads 533 are washed by moving the beads back and forth from blisters 544 and 546 via channel 543. Once the magnetic beads 533 are washed, the magnetic beads 533 are recaptured in blister 546 by activation of the magnet, and the wash solution is then moved to blister 522. This process may be repeated as necessary to wash the lysis buffer and sample debris from the nucleic acid-binding magnetic beads 533.

After washing, elution buffer stored at injection channel 515f is moved to blister 548, and the magnet is retracted. The solution is cycled between blisters 546 and 548 via channel 552, breaking up the pellet of magnetic beads 533 in blister 546 and allowing the captured nucleic acids to dissociate from the beads and come into solution. The magnet is once again activated, capturing the magnetic beads 533 in blister 546, and the eluted nucleic acid solution is moved into blister 548.

First-stage PCR master mix from injection channel 515g is mixed with the nucleic acid sample in blister 548. Optionally, the mixture is mixed by forcing the mixture between 548 and 564 via channel 553. After several cycles of mixing, the solution is contained in blister 564, where a pellet of first-stage PCR primers is provided, at least one set of primers for each target, and first-stage multiplex PCR is performed. If RNA targets are present, a reverse transcription (RT) step may be performed prior to or simultaneously with the first-stage multiplex PCR. First-stage multiplex PCR temperature cycling in the FilmArray® instrument is illustratively performed for 15-20 cycles, although other levels of amplification may be desirable, depending on the requirements of the specific application. The first-stage PCR master mix may be any of various master mixes, as are known in the art. In one illustrative example, the first-stage PCR master mix may be any of the chemistries disclosed in U.S. Pat. No. 9,932,634, herein incorporated by reference, for use with PCR protocols taking 20 seconds or less per cycle.

After first-stage PCR has proceeded for the desired number of cycles, the sample may be diluted, illustratively by forcing most of the sample back into blister 548, leaving only a small amount in blister 564, and adding second-stage PCR master mix from injection channel 515i. Alternatively, a dilution buffer from 515i may be moved to blister 566 then mixed with the amplified sample in blister 564 by moving the fluids back and forth between blisters 564 and 566. If desired, dilution may be repeated several times, using dilution buffer from injection channels 515j and 515k, or injection channel 515k may be reserved, illustratively, for sequencing or for other post-PCR analysis, and then adding second-stage PCR master mix from injection channel 515h to some or all of the diluted amplified sample. It is understood that the level of dilution may be adjusted by altering the number of dilution steps or by altering the percentage of the sample discarded prior to mixing with the dilution buffer or second-stage PCR master mix comprising components for amplification, illustratively a polymerase, dNTPs, and a suitable buffer, although other components may be suitable, particularly for non-PCR amplification methods. If desired, this mixture of the sample and second-stage PCR master mix may be pre-heated in blister 564 prior to movement to second-stage wells 582 for second-stage amplification. Such preheating may obviate the need for a hot-start component (antibody, chemical, or otherwise) in the second-stage PCR mixture.

In one embodiment, the illustrative second-stage PCR master mix is incomplete, lacking primer pairs, and each of the 102 second-stage wells 582 is pre-loaded with a specific PCR primer pair. In other embodiments, the master mix may lack other components (e.g., polymerase, Mg²⁺, etc.) and the lacking components may be pre-loaded in the array. If desired, second-stage PCR master mix may lack other reaction components, and these components may be pre-loaded in the second-stage wells 582 as well. Each primer pair may be similar to or identical to a first-stage PCR primer pair or may be nested within the first-stage primer pair. Movement of the sample from blister 564 to the second-stage wells 582 completes the PCR reaction mixture. Once high density array 581 is filled, the individual second-stage reactions are sealed in their respective second-stage blisters by any number of means, as is known in the art. Illustrative ways of filling and sealing the high density array 581 without cross-contamination are discussed in U.S. Pat. No. 8,895,295, already incorporated by reference. Illustratively, the various reactions in wells 582 of high density array 581 are simultaneously or individually thermal cycled, illustratively with one or more Peltier devices, although other means for thermal cycling are known in the art.

In certain embodiments, second-stage PCR master mix contains the dsDNA binding dye LCGreen® Plus (BioFire Diagnostics, LLC) to generate a signal indicative of amplification. However, it is understood that this dye is illustrative only, and that other signals may be used, including other dsDNA binding dyes and probes that are labeled fluorescently, radioactively, chemiluminescently, enzymatically, or the like, as are known in the art. Alternatively, wells 582 of array 581 may be provided without a signal, with results reported through subsequent processing.

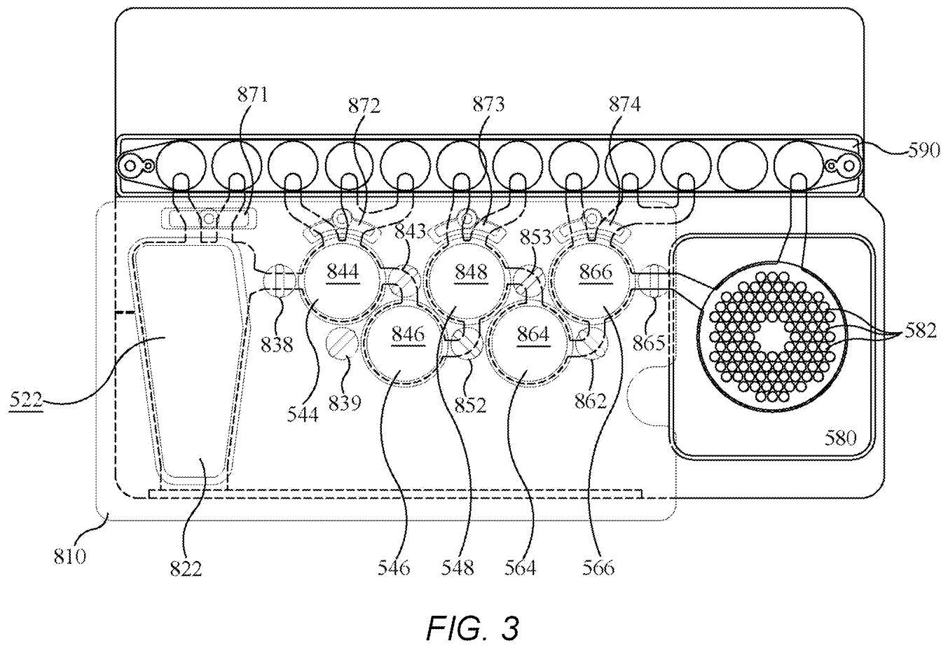

When pneumatic pressure is used to move materials within pouch 510, in one embodiment, a “bladder” may be employed. The bladder assembly 810, a portion of which is shown in FIGS. 2-3, includes a bladder plate 824 housing a plurality of inflatable bladders 822, 844, 846, 848, 864, and 866, each of which may be individually inflatable, illustratively by a compressed gas source. Because the bladder assembly 810 may be subjected to compressed gas and used multiple times, the bladder assembly 810 may be made from tougher or thicker material than the pouch. Alternatively, bladders 822, 844, 846, 848, 864, and 866 may be formed from a series of plates fastened together with gaskets, seals, valves, and pistons. Other arrangements are within the scope of this invention. Alternatively, an array or mechanical actuators and seals may be used to seal channels and direct movement of fluids between blisters. A system of mechanical seals and actuators that may be adapted for the instruments described herein is described in detail in US 2019-0344269, the entirety of which is already incorporated by reference.

Success of the secondary PCR reactions is dependent upon template generated by the multiplex first-stage reaction. Typically, PCR is performed using DNA of high purity. Methods such as phenol extraction or commercial DNA extraction kits provide DNA of high purity. Samples processed through the pouch 510 may require accommodations be made to compensate for a less pure preparation. PCR may be inhibited by components of biological samples, which is a potential obstacle. Illustratively, hot-start PCR, higher concentration of Taq polymerase enzyme, adjustments in MgCl₂ concentration, adjustments in primer concentration, addition of engineered enzymes that are resistant to inhibitors, and addition of adjuvants (such as DMSO, TMSO, or glycerol) optionally may be used to compensate for lower nucleic acid purity. While purity issues are likely to be more of a concern with first-stage amplification, it is understood that similar adjustments may be provided in the second-stage amplification as well.

When pouch 510 is placed within the instrument 800, the bladder assembly 810 is pressed against one face of the pouch 510, so that if a particular bladder is inflated, the pressure will force the liquid out of the corresponding blister in the pouch 510. In addition to bladders corresponding to many of the blisters of pouch 510, the bladder assembly 810 may have additional pneumatic actuators, such as bladders or pneumatically-driven pistons, corresponding to various channels of pouch 510. FIGS. 2-3 show an illustrative plurality of pistons or hard seals 838, 843, 852, 853, and 865 that correspond to channels 538, 543, 553, and 565 of pouch 510, as well as seals 871, 872, 873, 874 that minimize backflow into fitment 590. When activated, hard seals 838, 843, 852, 853, and 865 form pinch valves to pinch off and close the corresponding channels. To confine liquid within a particular blister of pouch 510, the hard seals are activated over the channels leading to and from the blister, such that the actuators function as pinch valves to pinch the channels shut. Illustratively, to mix two volumes of liquid in different blisters, the pinch valve actuator sealing the connecting channel is activated, and the pneumatic bladders over the blisters are alternately pressurized, forcing the liquid back and forth through the channel connecting the blisters to mix the liquid therein. The pinch valve actuators may be of various shapes and sizes and may be configured to pinch off more than one channel at a time. While pneumatic actuators are discussed herein, it is understood that other ways of providing pressure to the pouch are contemplated, including various electromechanical actuators such as linear stepper motors, motor-driven cams, rigid paddles driven by pneumatic, hydraulic or electromagnetic forces, rollers, rocker-arms, and in some cases, cocked springs. In addition, there are a variety of methods of reversibly or irreversibly closing channels in addition to applying pressure normal to the axis of the channel. These include kinking the bag across the channel, heat-sealing, rolling an actuator, and a variety of physical valves sealed into the channel such as butterfly valves and ball valves. Additionally, small Peltier devices or other temperature regulators may be placed adjacent the channels and set at a temperature sufficient to freeze the fluid, effectively forming a seal. Also, while the design of FIG. 1 is adapted for an automated instrument featuring actuator elements positioned over each of the blisters and channels, it is also contemplated that the actuators could remain stationary, and the pouch 510 could be transitioned such that a small number of actuators could be used for several of the processing stations including sample disruption, nucleic-acid capture, first and second-stage PCR, and processing stations for other applications of the pouch 510 such as immuno-assay and immuno-PCR. Rollers acting on channels and blisters could prove particularly useful in a configuration in which the pouch 510 is translated between stations. Thus, while pneumatic actuators are used in the presently disclosed embodiments, when the term “pneumatic actuator” is used herein, it is understood that other actuators and other ways of providing pressure may be used, depending on the configuration of the pouch and the instrument.

Turning back to FIG. 2, each pneumatic actuator is connected to compressed air source 895 via valves 899. While only several hoses 878 are shown in FIG. 2, it is understood that each pneumatic fitting is connected via a hose 878 to the compressed gas source 895. Compressed gas source 895 may be a compressor, or, alternatively, compressed gas source 895 may be a compressed gas cylinder, such as a carbon dioxide cylinder. Compressed gas cylinders are particularly useful if portability is desired. Other sources of compressed gas are within the scope of this invention. Similar pneumatic control may be provided, for example, for control of fluid movement in the pouches described herein, or other actuators, servos, or the like may be provided.

Several other components of the instrument are also connected to compressed gas source 895. A magnet 850, which is mounted on a second side 814 of support member 802, is illustratively deployed and retracted using gas from compressed gas source 895 via hose 878, although other methods of moving magnet 850 are known in the art. Magnet 850 sits in recess 851 in support member 802. It is understood that recess 851 can be a passageway through support member 802, so that magnet 850 can contact blister 546 of pouch 510. However, depending on the material of support member 802, it is understood that recess 851 need not extend all the way through support member 802, as long as when magnet 850 is deployed, magnet 850 is close enough to provide a sufficient magnetic field at blister 546, and when magnet 850 is fully retracted, magnet 850 does not significantly affect any magnetic beads 533 present in blister 546. While reference is made to retracting magnet 850, it is understood that an electromagnet may be used and the electromagnet may be activated and inactivated by controlling flow of electricity through the electromagnet. Thus, while this specification discusses withdrawing or retracting the magnet, it is understood that these terms are broad enough to incorporate other ways of withdrawing the magnetic field. It is understood that the pneumatic connections may be pneumatic hoses or pneumatic air manifolds, thus reducing the number of hoses or valves required. It is understood that similar magnets and methods for activating the magnets may be used in other embodiments.

The various pneumatic pistons 868 of pneumatic piston array 869 are also connected to compressed gas source 895 via hoses 878. While only two hoses 878 are shown connecting pneumatic pistons 868 to compressed gas source 895, it is understood that each of the pneumatic pistons 868 are connected to compressed gas source 895. Twelve pneumatic pistons 868 are shown.

A pair of temperature control elements are mounted on a second side 814 of support member 802. As used herein, the term “temperature control element” refers to a device that adds heat to or removes heat from a sample. Illustrative examples of a temperature control element include, but are not limited to, heaters, coolers, Peltier devices, resistive heaters, induction heaters, electromagnetic heaters, thin film heaters, printed element heaters, positive temperature coefficient heaters, and combinations thereof. A temperature control element may include multiple heaters, coolers, Peltiers, etc. In one aspect, a given temperature control element may include more than one type of heater or cooler. For instance, an illustrative example of a temperature control element may include a Peltier device with a separate resistive heater applied to the top and/or the bottom face of the Peltier. While the term “heater” is used throughout the specification, it is understood that other temperature control elements may be used to adjust the temperature of the sample.

As discussed above, first-stage heater 886 may be positioned to heat and cool the contents of blister 564 for first-stage PCR. As seen in FIG. 2, second-stage heater 888 may be positioned to heat and cool the contents of second-stage blisters of array 581 of pouch 510, for second-stage PCR. It is understood, however, that these heaters could also be used for other heating purposes, and that other heaters may be included, as appropriate for the particular application.

As discussed above, while Peltier devices, which thermocycle between two or more temperatures, are effective for PCR, it may be desirable in some embodiments to maintain heaters at a constant temperature. Illustratively, this can be used to reduce run time, by eliminating time needed to transition the heater temperature beyond the time needed to transition the sample temperature. Also, such an arrangement can improve the electrical efficiency of the system as it is only necessary to thermally cycle the smaller sample and sample vessel, not the much larger (more thermal mass) Peltier devices. For instance, an instrument may include multiple heaters (i.e., two or more) at temperatures set for, for example, annealing, extension, denaturation that are positioned relative to the pouch to accomplish thermal cycling. Two heaters may be sufficient for many applications. In various embodiments, the heaters can be moved, the pouch can be moved, or fluids can be moved relative to the heaters to accomplish thermal cycling. Illustratively, the heaters may be arranged linearly, in a circular arrangement, or the like. Types of suitable heaters have been discussed above, with reference to first-stage PCR.

When fluorescent detection is desired, an optical array 890 may be provided. As shown in FIG. 2, optical array 890 includes a light source 898, illustratively a filtered LED light source, filtered white light, or laser illumination, and a camera 896. Camera 896 illustratively has a plurality of photodetectors each corresponding to a second-stage well 582 in pouch 510. Alternatively, camera 896 may take images that contain all of the second-stage wells 582, and the image may be divided into separate fields corresponding to each of the second-stage wells 582. Depending on the configuration, optical array 890 may be stationary, or optical array 890 may be placed on movers attached to one or more motors and moved to obtain signals from each individual second-stage well 582. It is understood that other arrangements are possible. Some embodiments for second-stage heaters provide the heaters on the opposite side of pouch 510 from that shown in FIG. 2. Such orientation is illustrative only and may be determined by spatial constraints within the instrument. Provided that second-stage reaction zone 580 is provided in an optically transparent material, photodetectors and heaters may be on either side of array 581.

As shown, a computer 894 controls valves 899 of compressed air source 895, and thus controls all of the pneumatics of instrument 800. In addition, many of the pneumatic systems in the instrument may be replaced with mechanical actuators, pressure applying means, and the like in other embodiments. Computer 894 also controls heaters 886 and 888, and optical array 890. Each of these components is connected electrically, illustratively via cables 891, although other physical or wireless connections are within the scope of this invention. It is understood that computer 894 may be housed within instrument 800 or may be external to instrument 800. Further, computer 894 may include built-in circuit boards that control some or all of the components, and may also include an external computer, such as a desktop or laptop PC, to receive and display data from the optical array. An interface, illustratively a keyboard interface, may be provided including keys for inputting information and variables such as temperatures, cycle times, etc. Illustratively, a display 892 is also provided. Display 892 may be an LED, LCD, or other such display, for example.

Other instruments known in the art teach PCR within a sealed flexible container. See, e.g., U.S. Pat. Nos. 6,645,758, 6,780,617, and 9,586,208, herein incorporated by reference. However, including the cell lysis within the sealed PCR vessel can improve ease of use and safety, particularly if the sample to be tested may contain a biohazard. In the embodiments illustrated herein, the waste from cell lysis, as well as that from all other steps, remains within the sealed pouch. Still, it is understood that the pouch contents could be removed for further testing.

Turning back to FIG. 2, instrument 800 includes a support member 802 that could form a wall of a casing or be mounted within a casing. Instrument 800 may also include a second support member (not shown) that is optionally movable with respect to support member 802, to allow insertion and withdrawal of pouch 510. Illustratively, a lid may cover pouch 510 once pouch 510 has been inserted into instrument 800. In another embodiment, both support members may be fixed, with pouch 510 held into place by other mechanical means or by pneumatic pressure.

In the illustrative example, heaters 886 and 888 are mounted on support member 802. However, it is understood that this arrangement is illustrative only and that other arrangements are possible. Illustrative heaters include Peltiers and other block heaters, resistive heaters, electromagnetic heaters, and thin film heaters, as are known in the art, to thermocycle the contents of blister 864 and second-stage reaction zone 580. Bladder plate 810, with bladders 822, 844, 846, 848, 864, 866, hard seals 838, 843, 852, 853, and seals 871, 872, 873, 874 form bladder assembly 808, which may illustratively be mounted on a moveable support structure that may be moved toward pouch 510, such that the pneumatic actuators are placed in contact with pouch 510. When pouch 510 is inserted into instrument 800 and the movable support member is moved toward support member 802, the various blisters of pouch 510 are in a position adjacent to the various bladders of bladder assembly 810 and the various seals of assembly 808, such that activation of the pneumatic actuators may force liquid from one or more of the blisters of pouch 510 or may form pinch valves with one or more channels of pouch 510. The relationship between the blisters and channels of pouch 510 and the bladders and seals of assembly 808 is illustrated in more detail in FIG. 3.

### Isolation, Concentration, Characterization, and/or Identification of Microorganisms in a Sample

The present invention provides methods, systems, and apparatuses for isolating, concentrating, characterizing and/or identifying microorganisms in a sample. In one embodiment, the microorganism is a bacterium. In another embodiment, the microorganism is a fungal organism (e.g., a yeast or a mold). In a further embodiment, the microorganism is a parasite. In another embodiment, the microorganism may be a combination of microorganisms selected from the group consisting of bacteria, yeasts, molds, and parasites. The methods, systems, and apparatuses may be particularly useful for the separation, characterization and/or identification of microorganisms from complex samples such as blood, urine, or cerebrospinal fluid. In a preferred aspect, the methods, systems, and apparatuses of the present invention may be used for isolating, characterizing and/or identifying microorganisms direct from blood in order, for example, to rapidly determine whether a patient is septic or pre-septic.

As used herein, “direct from blood” or “direct from whole blood” in reference to determining the presence of microorganisms present in a blood sample means determining the presence of microorganisms by concentrating and/or isolating microorganisms from whole blood and then identifying the microorganisms. “Whole blood” is blood (e.g., human blood) as you would find in a circulatory system with none of its components separated or removed. Blood with an added anticoagulant is generally still referred to as whole blood. Microorganisms suitably may be concentrated and/or isolated from whole blood without using a pre-concentration and/or pre-isolation blood culture step to increase the numbers of microorganisms in the sample. Microorganisms suitably may be concentrated and/or isolated from blood following a brief (e.g., <5 hrs, <4 hrs, <3 hrs, <2 hrs, or <1 hr) culturing step to increase the numbers of microorganisms in the sample. Subsequent to concentrating and/or isolating the microorganisms, the microorganisms suitably may be identified by a number of techniques including, but not limited to, one or more of a molecular test (i.e., a nucleic acid-based test), a phenotypic test, a proteomic test, an optical test, or a culture-based test. Subsequent to concentrating and/or isolating the microorganisms, the microorganisms suitably may be briefly (<5 hrs, <4 hrs, <3 hrs, <2 hrs, or <1 hr (e.g., 3 hrs)) cultured to increase the numbers in the concentrated/isolated fraction. However, culturing suitably may not be needed in the methods and systems described herein. For example, the methods described herein may suitably work for all bacterial and fungal organisms of interest, including, but not limited to, fastidious organisms that do not typically grow well or quickly in blood culture, aerobic and anaerobic organisms that may require different culturing conditions, and organisms that may need different media formulations for growth and detection.

Characterization and/or identification of the microorganisms in a concentrated sample of microorganism (e.g., a centrifugation pellet) suitably may not involve identification of an exact species. Characterization encompasses the broad categorization or classification of biological particles as well as the actual identification of a single species. As used herein, “identification” means determining to which family, genus, species, and/or strain a microorganism belongs to. For example, identifying a microorganism isolated from a biological sample (e.g., blood, urine, or cerebrospinal fluid) to the family, genus, species, and/or strain level.

The methods, systems, and apparatuses described herein allow for the characterization and/or identification of microorganisms more quickly than prior techniques, resulting in faster diagnoses (e.g., in a subject having or suspected of having sepsis). The steps involved in the methods of the invention, from obtaining a sample to characterization/identification of microorganisms, can be carried out in a very short time frame to obtain clinically relevant actionable information. In certain embodiments, the methods of the invention can be carried out in less than about 120 minutes, e.g., in less than about 110, 100, 95, 90, 85, 80, 75, 70, 65, 60, 55, 50, 45, 40, 35, 30, 25, 20, 15, 10, 5, 4, 3, 2, 1 minute, or any range of or between or encompassing the foregoing time points. In a preferred embodiment, the methods of the invention can be carried out in less than about 90 minutes (e.g., about 75 minutes). For example, the elapsed time from when a whole blood sample is collected from a patient suspected of having sepsis to the completion of analysis and positive identification of the infectious agent (if present) may, in many scenarios, be less than about 90 minutes. As more rapid molecular analysis systems become available, the sample-to-answer time may be reduced considerably. While sepsis and whole blood are used in the previous example, similar times may be achievable for other complex sample types where low titer organisms are concentrated from a large volume of blood or other sample types. The tremendous rapidity of the methods of the invention represents an improvement over prior methods. The methods can be used to characterize and/or identify any microorganism as described herein.

The illustrative workflow associated with the methods, systems, and apparatuses described herein is simple and minimizes handling of the sample, the lysate, and the microorganisms. For instance, the sample can be mixed with a differential lysis buffer in a single tube for lysis and isolation of the microorganisms. In one embodiment, the microorganisms may be recovered from a single tube in a manner that sequesters the microorganism pellet from the lysate, thereby reducing the risk of handling potentially infectious materials and/or contaminating the samples. Additionally, the methods of the invention can be fully automated, which further reduces the risk of handling infectious materials and/or contaminating the samples.

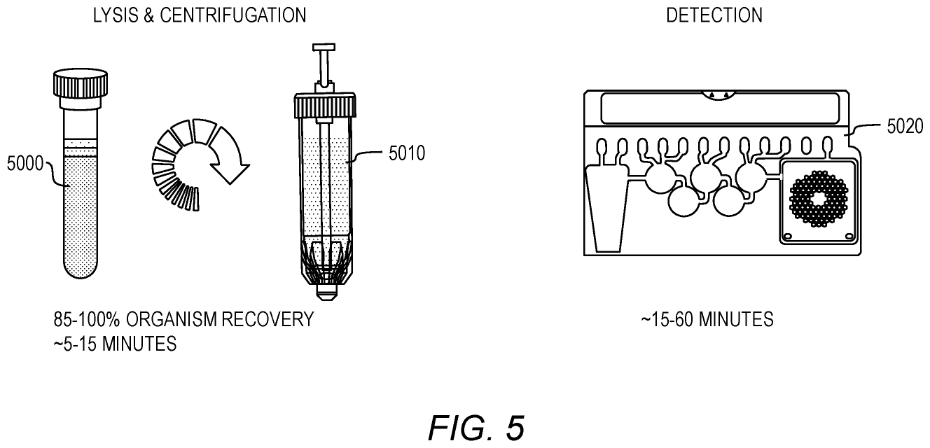

FIG. 5 is a schematic illustration of one embodiment of components useful for the methods, systems, and apparatuses described herein. The illustrated method includes a limited number of components, a limited number of steps, and can be completed in about 20-75 minutes from contact of the sample and the differential lysis buffer, to provide a simplified workflow and shorter time-to-results. The illustrated method of FIG. 5 includes steps of obtaining a sample 5000 (e.g., a whole blood sample, a urine sample, a cerebrospinal fluid sample, or an environmental sample), preparing a lysate, recovering the microorganism cells from the lysate, and characterizing and/or identifying microorganisms in a sample. In one embodiment, the sample 5000, which may be a blood sample, may be provided in a standard blood collection tube (e.g., a vacutainer or the like) with or without anticoagulants. In one embodiment, the sample 5000 and a differential lysis buffer may be combined for lysis of substantially all (e.g., >90%) of the non-microorganism cells in the sample 5000. In the illustrated embodiment, the lysate may be prepared in a specially designed centrifugal concentrator 5010. In one embodiment, the differential lysis buffer may be provided in the centrifugal concentrator 5010 and lysis of non-microorganism cells in the sample may be initiated simply by adding the sample 5000 to the centrifugal concentrator 5010, thereby combining the sample and the differential lysis buffer in the centrifugal concentrator 5010. In another embodiment, the sample 5000 is mixed with the differential lysis buffer and then disposed into the centrifugal concentrator 5010, e.g., pipetted as a mixture into the centrifugal concentrator. After combining, the differential lysis buffer and the sample are combined for a period of time (e.g., 1-5 minutes) to yield a lysate. In one embodiment, the microorganisms may be recovered from the lysate by centrifugation, filtration, or the like. In the case of centrifugation, the microorganism cells may be pelleted in the centrifugal concentrator 5010 by centrifuging the centrifugal concentrator for a period of time in a range of about 4-10 minutes at about 1,000×g to about 20,000×g. In the illustrated embodiment, the recovered microorganism cells may be added from the centrifugal concentrator 5010 into an analysis device 5020 that is configured for characterizing and/or identifying microorganisms in the sample at clinically relevant levels. Characterizing and/or identifying microorganisms in the illustrated analysis device 5020 can be performed rapidly (e.g., about 15-60 minutes). However, the illustrated analysis device is merely illustrative. For example, in some embodiments, the microorganisms may be characterized and/or identified by sequencing (e.g., next-generation sequencing).

Samples

Samples that may be tested by the methods and systems described herein may include both clinical and non-clinical samples in which microorganism presence and/or growth is or may be suspected, as well as samples of materials that are routinely or occasionally tested for the presence of microorganisms. The amount of sample utilized may vary greatly due to the versatility and/or sensitivity of the method. One advantage of the methods and systems described herein is that complex sample types, such as, e.g., blood, bodily fluids, and/or other opaque substances, may be tested directly utilizing the system with little or no extensive pretreatment.

By “sample” is meant an animal; a tissue or organ from an animal, including, but not limited to, a human animal; a cell (either within a subject (e.g., a human or non-human animal), taken directly from a subject, or a cell maintained in culture or from a cultured cell line); a cell lysate (or lysate fraction) or cell extract; a solution containing one or more molecules derived from a cell, cellular material, or viral material (e.g. a polypeptide or nucleic acid); or a solution containing a non-naturally occurring nucleic acid, which is assayed as described herein. Samples that may be tested by the methods and systems described herein may include both clinical and non-clinical samples in which microorganism presence and/or growth is or may be suspected, as well as samples of materials that are routinely or occasionally tested for the presence of microorganisms. Clinical samples that may be tested include any type of sample typically tested in clinical or research laboratories, including, but not limited to, blood, serum, plasma, blood fractions, joint fluid, urine, semen, saliva, feces, cerebrospinal fluid, gastric contents, vaginal secretions, tissue homogenates, bone marrow aspirates, bone homogenates, sputum, aspirates, swabs and swab rinsates, other body fluids, blood products (e.g., platelets, serum, plasma, white blood cell fractions, etc.), donor organ or tissue samples, and the like. Some specimen samples that may be cultured and subsequently tested may include blood, serum, plasma, platelets, red blood cells, white blood cells, blood fractions, joint fluid, urine, nasal samples, semen, saliva, feces, cerebrospinal fluid, gastric contents, vaginal secretions, tissue homogenates, bone marrow aspirates, bone homogenates, sputum, aspirates, swabs and swab rinsates, other body fluids, and the like. For example, it may be an option in some embodiments to subject a sample like blood from a subject to a limited culture step (e.g., in a range of 1 minute to 4 hours) prior to testing to increase the levels of detectable microorganisms in the sample. In another option, a sample like blood may be cultured prior to selective lysis and recovery of microorganism, a microorganism may be cultured during lysis and recovery, or microorganism may be cultured from a pellet recovered (e.g., by centrifugation) from a selectively lysed sample (e.g., by growing organisms is a liquid medium or on a solid plate). The culturing of microorganisms (particularly bacteria and fungi) suitably may be faster when the cell concentration is higher. Culturing from a recovered or concentrated microorganism (e.g., from a pellet obtained from a centrifugation step) may suitably be faster than blood culture. Suitably culturing from a recovered or concentrated microorganism may also remove antibiotics and defensins that may be present in blood, which may also promote faster growth.

The present invention finds use in research as well as veterinary and medical applications. Suitable subjects from which clinical samples can be obtained are generally mammalian subjects, but can be any animal. The term “mammal” as used herein includes, but is not limited to, humans, non-human primates, cattle, sheep, goats, pigs, horses, cats, dog, rabbits, rodents (e.g., rats or mice), etc. Human subjects include neonates, infants, juveniles, adults and geriatric subjects. Subjects from which samples can be obtained include, without limitation, mammals, birds, reptiles, amphibians, and fish.

Non-clinical samples that may be tested also include substances, encompassing, but not limited to, foodstuffs, beverages, pharmaceuticals, cosmetics, water (e.g., drinking water, non-potable water, and waste water), seawater ballasts, air, soil, sewage, plant material (e.g., seeds, leaves, stems, roots, flowers, fruit), biowarfare samples, and the like. Samples may also include environmental samples such as, but not limited to, soil, air monitoring system samples (e.g., material captured in an air filter medium), surface swabs, and vectors (e.g., mosquitos, ticks, fleas, etc.). The method is also particularly well suited for real-time testing to monitor contamination levels, process control, quality control, and the like in industrial settings. In a preferred embodiment of the invention, samples are obtained from a subject (e.g., a patient) having or suspected of having a microbial infection. In one embodiment, the subject has or is suspected of having septicemia, e.g., bacteremia or fungemia. Preferably, the sample may be a blood sample that is tested directly after being collected from the subject. That is, the sample is a whole blood sample that has not been added to a blood culture medium and that has not been treated or cultured or diluted prior to testing. In another embodiment, the sample may be from a blood culture grown from a sample of the patient's blood, e.g., a BacT/ALERT® blood culture. The blood culture sample may be from a positive blood culture, e.g., a blood culture that indicates the presence of a microorganism. In certain embodiments, the sample may be taken from a positive blood culture within a short time after it turns positive, e.g., within about 6 hours, e.g., within about 5, 4, 3, or 2 hours, or within about 60 minutes, e.g., about 55, 50, 45, 40, 35, 30, 25, 20, 15, 10, 5, 4, 3, 2, or 1 minute. In one embodiment, the sample may be taken from a culture in which the microorganisms are in log phase growth. In another embodiment, the sample may be taken from a culture in which the microorganisms are in a stationary phase. In some embodiments, the whole blood sample may be provided as part of the method within 1 hour of the whole blood sample being taken from the patient. In yet another embodiment, the sample may be or may include blood that has been cultured for a period of time (e.g., in a range of 1 minute to 4 hours) less than the time typically needed to yield a positive blood culture result. In some embodiments, the sample is provided at room temperature for use in the method, while in other embodiments, the sample is cooled after being obtained from the patient before being provided for use in the method. For example, the sample may be refrigerated after being obtained from the patient until the method can be performed.

The present invention provides high sensitivity for the detection and identification of microorganisms. Illustratively, this enables detection and identification of microorganisms without first having to go through the steps of liquid culture, followed by isolating microorganisms and growing them on a solid or semisolid medium, and sampling the colonies that grow. Thus, in one embodiment of the invention, the sample is not from a liquid culture or a microbial (e.g., bacteria, yeast, or mold) colony grown on a solid or semisolid surface. In order to expedite identification of a possible BSI, in some embodiments, the method includes the step of lysing the sample without culturing the sample after obtaining it from a patient. In some embodiments, the methods described herein may be used even in a patient has been treated with antimicrobials prior to blood sample collection. Patients presenting in a hospital with symptoms consistent with sepsis are often started on antimicrobials immediately, before sepsis can be definitively ruled in or ruled out. While such treatment protocols consistent with the standard of care, antimicrobials can interfere with the blood culture that is used in classical sepsis diagnosis. Surprisingly, the methods described herein can still be used to diagnose sepsis in patients on antimicrobial treatment if intact microbial cells are still present in the blood.

The volume of the sample should be sufficiently large to produce a pellet of microorganisms which can be analyzed after the separation step of the methods of the invention is carried out. Appropriate volumes will depend on the source of the sample, the anticipated level of microorganisms in the sample, and the analysis method employed for characterization and identification of the microorganisms. For example, whole blood from a patient with BSI typically has a microorganism load of ˜1-100 cfu/ml (e.g., <1-10 cfu/ml). In general, the sample size can be about 50, 40, 30, 20, 15, 10, 5, 4, 3, or 2 ml (e.g., about 10 ml). In certain embodiments, the sample size can be about 1 ml, e.g., about 0.75, 0.5, or 0.25 ml. In certain embodiments in which the separation is carried out on a microscale, the sample size can be less than about 200 μl, e.g., less than about 150, 100, 50, 25, 20, 15, 10, or 5 μl. In some embodiments (e.g., when the sample is expected to comprise a small number of microorganisms), the sample size can be about 100 ml or more, e.g., about 250, 500, 750, or 1000 ml or more. A positive blood culture will contain a higher level of microorganisms per ml, so a smaller volume of blood culture medium may be used as compared to whole blood.

While much of the discussion herein relates specifically to whole blood, the methods, systems, and apparatuses described herein may be used for other sample types, as noted above in the definition of “sample.” Two specific examples of additional sample types are urine and cerebrospinal fluid (CSF). Urine and CSF often contain white blood cells (WBCs) during infection, which can harbor intracellular pathogens. These WBCs may be produced in fighting the infection or, in the case of CSF, many pathogens gain access to the brain/spinal column by hiding inside WBCs or other blood cells, which are then able to pass the blood-brain barrier. By selectively lysing the pathogen-harboring blood cells (and not the pathogen cells) with the differential lysis buffer disclosed herein, the pathogens in the cells can be released and can be concentrated in a pellet that may be substantially free of contaminating eukaryotic host DNA (e.g., contaminating host DNA may be reduced >95%). Additionally, epithelial cells of the bladder may exfoliate during infection to expel pathogen-laden cells and as a preventative measure to prevent the infection from spreading. Like the white blood cells, these epithelial cells can be lysed by the differential lysis buffer disclosed herein and the intact pathogen cells can be concentrated in a pellet without contamination from the bladder cells.

As discussed in greater detail elsewhere herein, the recovered pathogen cells can be lysed and the nucleic acids from the pathogen cells can be recovered for analysis. Because the pathogen cells are isolated without significant host cell DNA contamination, the recovered pathogen nucleic acids are suitable for downstream molecular assays for characterizing and/or identifying the pathogens. In some embodiments, the pathogen cells may be used for downstream characterization and/or identification of the pathogens by molecular methods (e.g., by PCR amplification of pathogen DNA or RNA and identification of amplicons), genetic sequencing (e.g., by a next-generation sequencing technique), or by mass spectrometry. The devices and methods described herein can remove many or all of the host cellular components so that the pathogen signal can be discerned by any of these methods.

Lysis Step

The next step in illustrative methods of the invention after providing or obtaining a sample is to lyse non-microbial cells that may be present in the sample, e.g., blood cells and/or tissue cells or other eukaryotic host cells. In some embodiments, the method includes selectively lysing cells to permit separation of microorganisms from other components of the sample. The separation of microorganisms from other components reduces interference during later interrogation step(s). If non-microorganism cells are not expected to be present in the sample or not expected to interfere with the interrogation step, the lysis step may be omitted. In one embodiment, the cells to be lysed are non-microorganism cells that are present in the sample and few or no microorganism cells that may be present in the sample are lysed. However, in some embodiments, the selective lysing of specific classes of microorganisms may be desirable and thus can be carried out according to the methods described herein and as are well known in the art. For example, a class of undesired microorganisms can be selectively lysed, e.g., yeasts are lysed while bacteria are not, or vice versa. In another embodiment, the desired microorganisms are lysed in order to separate a particular subcellular component of the microorganisms, e.g., cell membranes or organelles. In one embodiment, all of the non-microbial cells are lysed. In other embodiments, a portion of the non-microbial cells are lysed, e.g., enough cells to prevent interference with the interrogation step. The lysing of cells may be carried out by any method known in the art to be effective to selectively lyse cells with or without lysing microorganisms, including, without limitation, addition of a differential lysis buffer, sonication, and/or osmotic shock.

A differential lysis buffer is one that is capable of selectively lysing one class of cells, e.g., non-microorganism cells (e.g., by solubilizing eukaryotic cell membranes) and/or some microorganism cells and not lysing another class of cells, e.g. microorganisms or a type of microorganisms. In one embodiment, the differential lysis buffer can include an aqueous medium, one or more detergents, a buffering substance, one or more salts, and can further include additional agents. In one embodiment, the differential lysis buffer may further include one or more enzymes (e.g., a protease). In one embodiment, the detergent can be a non-denaturing lytic detergent, such as Triton® X-100 Triton® X-100-R, Triton® X-114, NP-40, Igepal® CA 630, Arlasolve™200, Brij O10 (also known as Oleth-10, Brij 96V, Brij 97, Volpo 10 NF, Volpo N10) (the Brij name is a registered trademark of Croda International Plc), CHAPS, octyl β-D-glucopyranoside, saponin, and nonaethylene glycol monododecyl ether (aka, C12E9, polidocenol, Brij 35). In one embodiment, the detergent can be a non-ionic surfactant. Examples of suitable non-ionic surfactants include, but are not limited to, Triton X-114, NP-40, Arlasolve 200, Brij O10, octyl β-D-glucopyranoside, a saponin, nonaethylene glycol monododecyl ether, and combinations thereof. In a preferred embodiment, the non-ionic surfactant is a polyoxyethyene ether (POE ether). POE ethers are a class of non-ionic surfactants that may be used for cell membrane disruption. POE ethers consist of an alkyl chain, a hydrophilic portion comprised of ‘n’ oxyethylene units, and a terminal —OH group. Suitable examples of POE ethers include, but are not limited to, Arlasolve 200 (Poly(Oxy-1,2-Ethanediyl)), Brij O10 (and other Brij detergents), and nonaethylene glycol monododecyl ether (Brij 35). Optionally, denaturing lytic detergents can be included, such as sodium dodecyl sulfate, N-laurylsarcosine, sodium deoxycholate, bile salts, hexadecyltrimethylammonium bromide, SB3-10, SB3-12, amidosulfobetaine-14, and C7BzO. Optionally, solubilizers can also be included, such as Brij 98, Brij 58, Brij 35, Tween® 80, Tween® 20, Pluronic® L64, Pluronic® P84, non-detergent sulfobetaines (NDSB 201), amphipols (PMAL-C8), and methyl-β-cyclodextrin. Typically, non-denaturing detergents and solubilizers are used at concentrations above their critical micelle concentration (CMC), while denaturing detergents may be added at concentrations below their CMC. For example, non-denaturing lytic detergents can be used at a concentration of about 0.010% to about 10%, e.g., about 0.015% to about 1.0%, e.g., about 0.05% to about 0.5%, e.g., about 0.10% to about 0.30% (final concentration after dilution with the sample). Enzymes that can be used in the differential lysis buffer include, without limitation, enzymes that digest nucleic acids and other membrane-fouling materials (e.g., proteinase XXIII, DNase, neuraminidase, polysaccharidase, Glucanex®, and Pectinex®). In a specific embodiment, the differential lysis buffer does not include DNase and is not used in combination with DNase. Other additives that can be used include, without limitation, reducing agents such as 2-mercaptoethanol (2-Me) or dithiothreitol (DTT) and stabilizing agents such as magnesium, pyruvate, and humectants.

The differential lysis buffer can be buffered at any pH that is suitable to lyse the desired cells, and will depend on multiple factors, including without limitation, the type of sample, the cells to be lysed, and the detergent used. In some embodiments, the pH can be in a range from about 2 to about 13, e.g., about 6 to about 10, e.g., about 7 to about 9, e.g., about 7 to about 8. Suitable pH buffers may include any buffer capable of maintaining a pH in the desired range. In some embodiments, buffers may be used outside their pH buffering range. Suitable examples of buffering substances may include, but are not limited to, about 0.005 M to about 1.0 M CAPS, CAPSO, CHES, CABS, and combinations thereof. In a specific example, the differential lysis buffer has a composition shown below in Table 1.

TABLE 1

| | | | | | Total | Lysis |
|---|---|---|---|---|---|---|
| Differential | | Brij | | 0.45% | Volume | buffer:Blood |
| Lysis Buffer | CAPS | O10 | pH | NaCl | (mL) | ratio |
| Concentration | 13.3 mMol | 0.33% | 10.2-10.5 | 0.45% | 30 mL | 3:1 |
| of Buffer | | | | | | |
| Final | 10 mMol | 0.25% | 7.6-8.0 | ~0.34% | 40 mL | |
| Concentration | | | | | | |
| (+10 mL | | | | | | |
| whole blood) | | | | | | |

In the specific example illustrated in Table 1, the sample is ˜10 ml of whole blood, which is combined with ˜30 ml of the differential lysis buffer.

CAPS is the buffering substance; CAPS has a pKa at 25° C. of about 10.4 and a typical buffering range of −9.7-11.1. Prior to combining the differential lysis buffer with the blood sample the CAPS buffer is in its buffering range. However, after combining the differential lysis buffer with the blood sample, the CAPS buffer in this example is well outside of its buffering range (e.g., at a pH of about 7-8). Surprisingly, it has been found that using CAPS (and chemically similar buffers—e.g., CAPSO, CHES, and CABS) in the differential lysis buffer that is outside of its buffering range can have a synergistic effect that improves the lysis. Without being tied to one theory, it is believed that CAPS may be acting like a second detergent to help permeablize and lyse the non-microorganism cells. For instance, at a pH of ˜7-8 (e.g., a pH of 7.6-8), CAPS buffer will be almost fully protonated and positively charged. According to the Henderson-Hasselbach equation, for example, at the example pH range of ˜7.0-8.0, the ratio of protonated to unprotonated CAPS species will be about 250:1 or greater. For CAPS buffer at a pH of ˜7.0-8.0, a protonated to unprotonated ratio of about 250:1 or greater is an example of what it means to be “substantially positively charged.” Cell membranes generally have a net negative charge, so it is theorized that the positive charge on the CAPS buffer could attract the CAPS molecules to the surface of the cells. CAPS has a phenyl ring that can insert into the hydrophobic membranes of the non-microorganism cells to help permeablize the cells. CAPSO, CHES, and CABS have a similar structure to CAPS and it is expected that CAPSO, CHES, and CABS and combinations of CAPS, CAPSO, CHES, and CABS and similar buffers could provide similar results. CAPSO has a pKa at 25° C. of about 9.6 and a typical buffering range of ˜8.9-10.3, CHES has a pKa at 25° C. of about 9.3 and a typical buffering range of ˜8.6-10, and CABS has a pKa at 25° C. of about 10.7 and a typical buffering range of ˜10-11.4. For CAPSO, CHES, and CABS, the Henderson-Hasselbach equation provides that at the example pH range of ˜7.0-8.0, the ratio of protonated to unprotonated buffer species will be in a range of about 500:1 or greater (i.e., CABS at pH ˜7.0-8.0), about 40:1 or greater (i.e., CAPSO at pH ˜7.0-8.0), and 20:1 or greater (i.e., CHES at pH ˜7.0-8.0). Accordingly, for CAPSO, CHES, and CABS at a pH of about 7.0-8.0, a ratio of protonated to unprotonated CAPSO of about 40:1 or greater, a ratio of protonated to unprotonated CHES of about 20:1 or greater, and a ratio of protonated to unprotonated CABS of about 500:1 or greater, respectively, are further examples of what it means to be “substantially positively charged.” A person of ordinary skill will also appreciate that the buffering substance used in the differential lysis buffer suitably may include a combination of CAPS, CAPSO, CHES, and CABS. For such a combination, any ratio of protonated to unprotonated species of about 20:1 or greater is a further example of what it means to be “substantially positively charged.”

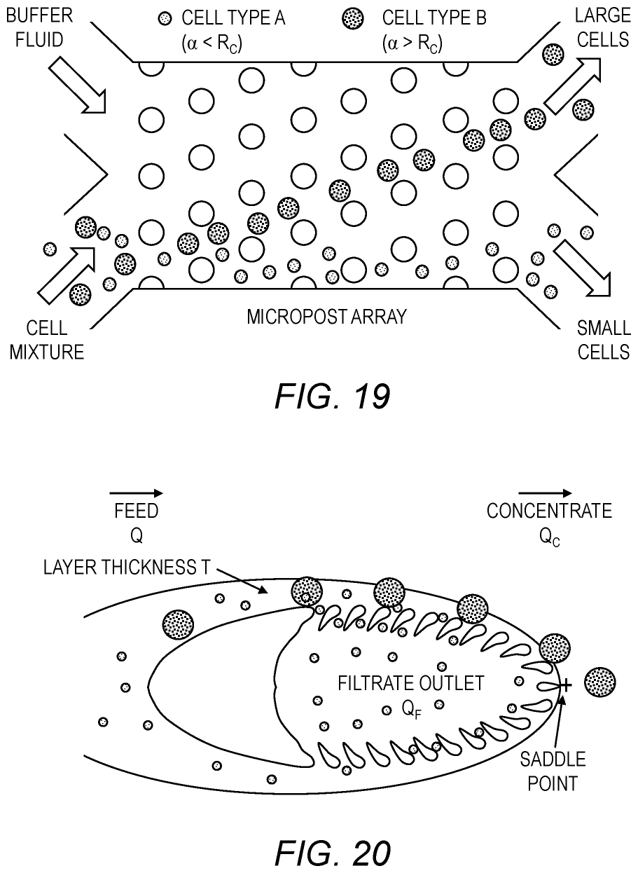

In one embodiment, the sample and the differential lysis buffer are combined for a sufficient time for lysis to occur, e.g., about 1, 2, 3, 4, 5, 10, 15, 20, 25, 30, 40, 50, or 60 seconds, or about 2, 3, 4, 5, 6, 7, 8, 9, 10, 15, or 20 minutes or longer, e.g., about 1 second to about 20 minutes, about 1 second to about 5 minutes, or about 1 second to about 2 minutes. In one embodiment, up to 50%, 60%, 70%, 80%, 90%, 95%, 97%, 98%, 99% on non-microbial cells in the sample may be lysed within 2-5 minutes of combining the sample with the differential lysis buffer. In some embodiments, the sample and differential lysis buffer are combined for sufficient time for solubilization of cell membranes to occur. Solublization of cell membranes of blood cells (i.e., non-microbial cells) is illustrated in FIG. 21. The OD500 absorbance (500 nm wavelength) of whole blood combined with several differential lysis buffer formulations was measured at various time points to show the efficacy of lysis of the blood cells at room-temperature. The absorbance drop as a function of time illustrates the progress of lysis. The buffer/blood combination containing 0.125% BrijO10 and 50 mM CAPS (◯) (i.e., concentration of detergent and buffer after combining 10 ml of whole blood with 30 ml of differential lysis buffer) was ineffective for lysis, possibly due to insufficient detergent concentration. The other three buffers tested (0.15% BrijO10 and 100 mM CAPS (Δ), 0.25% BrijO10 and 10 mM CAPS (□), and 0.25% BrijO10 and 50 mM CAPS(⋄)) were each effective for lysis of blood cells. The absorbance drop in these buffer/blood combinations illustrates that lysis was complete in the 100 mM and 50 mM CAPS buffers within 2 minutes and the within 3 minutes for the 10 mM CAPS buffer. The buffer containing 0.25% BrijO10 and 10 mM CAPS is the buffer illustrated above in Table 1. As illustrated in FIG. 21, the lysis time will depend on the strength of the differential lysis buffer, e.g., the concentration of the detergent and/or pH of the solution. In general, it is expected that milder lysis buffers will require more time and a greater dilution of the sample to fully or partially solubilize non-microbial cells. The strength of the differential lysis buffer can be selected based on the microorganisms known to be or suspected to be in the sample. For microorganisms that are more susceptible to lysis, a mild differential lysis buffer can be used. The lysis can take place at a temperature of about 2° C. to about 45° C., e.g., about 15° C. to about 40° C., e.g., about 20° C. to about 40° C.

In one embodiment, the differential lysis buffer can be loaded into a syringe and the sample can then be aspirated into the syringe such that the combining occurs within the syringe. In one embodiment, the sample and the differential lysis buffer can be provided in separate tubes and they can be combined by pouring one into the other. In one embodiment, the differential lysis buffer can be provided in a centrifugal concentrator and the sample can be aspirated into the centrifugal concentrator such that the combining and microorganism recovery occur within the centrifugal concentrator. In some embodiments, mixing occurs by combining the sample and the differential lysis buffer in solution. In a further embodiment, mixing includes agitating the combined sample and differential lysis buffer. For example, the sample and the differential lysis buffer may be combined in the centrifugal concentrator and mixed by tipping or gently shaking the centrifugal concentrator. In another example, a bead beater or sonicator may be used to agitate the combined sample and differential lysis buffer.

In some embodiments, the lysis conditions (e.g., the combining and/or the combining time), as well as the separation and/or interrogation steps, can be sufficient to kill some or all of the microorganisms in the sample. The methods of the present invention are highly versatile and do not require that the microorganisms be viable for the isolation and identification to occur. In certain embodiments, some or all of the microorganisms may be dead, with death occurring before, during, and/or after the steps of the methods being carried out. In other embodiments, some or all of the microorganisms may be alive at the conclusion of the separation step such that further culturing of the microorganism in an appropriate culture media (e.g., bacterial media or fungal media) at a culturing temperature (e.g., about 37° C. for bacteria and about 32° C. for many fungal species) is possible. For example, the microorganisms may be alive after the separation step and then included in a separate technique for determining whether the microorganisms are susceptible or resistant to one or more antibiotics. suitably, growth of the microorganisms may not be affected by the use of the differential lysis buffer.

Separation Step

After the sample has been lysed, a separation step can be carried out to separate the microorganisms from other components of the sample and to concentrate the microorganisms into a pellet that can be interrogated for identification and characterization purposes. The separation does not have to be complete, i.e., it is not required that 100% separation occur. Illustratively, the separation of the microorganisms from other components of the sample is sufficient to permit interrogation of the microorganisms without substantial interference from the other components. For example, the separation can result in a microorganism pellet that is at least about 10, 20, 30, 40, 50, 60, 70, 80, 90, 95, 96, 97, 98, or 99% pure or higher. One contaminant that potentially confounds microorganism identification direct from whole blood is human genomic DNA. In one example aspect, the inventors in the present case have found that treatment of whole blood with the differential lysis buffer described herein is capable of removing 98% or more of human genomic DNA when microorganisms are subsequently pelleted from blood lysate, even without a DNase treatment.

In one embodiment, the separation is carried out by a centrifugation step in which the sample (e.g., a lysed sample) is placed in a centrifugal concentrator and the centrifugal concentrator container is centrifuged under conditions in which the microorganisms pellet at the bottom and/or sides of the container and other components of the sample (e.g., lysed cell components) in the sample medium stay in the supernatant. This separation isolates the microorganisms away from materials in the sample, such as culture medium, cell debris, human genomic DNA, and/or other components that might interfere with interrogation of the microorganisms (e.g., by amplification and detection of microorganism-specific nucleic acids). This separation isolates the microorganisms from the bulk volume of sample and reduces the volume of the microorganism portion and concentrates the microorganism in a small volume (e.g., ˜200 μl). In one embodiment, the differential lysis buffer is provided in the centrifugal concentrator and lysis is initiated by combining the sample and the differential lysis buffer for a period of time sufficient for lysis, and then recovery of the microorganisms by centrifugation. In one embodiment, the centrifugal concentrator does not include a density cushion, a physical separator, or a similar medium known in the art. Unexpectedly, it has been found that a density cushion is not necessary to provide adequate separation and isolation of the microorganism from contaminating debris when used with a molecular technique for identification or characterization.

In one embodiment of the invention, the centrifugal concentrator is centrifuged in a swinging bucket rotor so that the microorganisms form a pellet directly on the bottom of the tube. The container is centrifuged at a sufficient acceleration and for a sufficient time for the microorganisms to pellet and/or be separated from other components of the sample. The centrifugation acceleration illustratively can be about 1,000×g to about 20,000×g, e.g., about 2,500×g to about 15,000×g, e.g., about 7,500×g to about 12,500×g, etc. The centrifugation time illustratively can be about 30 seconds to about 30 minutes, e.g., about 1 minute to about 15 minutes, e.g., about 1 minute to about 10 minutes. The centrifugation illustratively can be carried out at a temperature of about 2° C. to about 45° C., e.g., about 15° C. to about 40° C., e.g., about 20° C. to about 30° C. In one embodiment, the centrifugal concentrator comprises a closure, and the closure is applied to the container to form a seal prior to centrifugation. The presence of a closure decreases the risks from handling microorganisms that are or may be infectious and/or hazardous, as well as the risk of contaminating the sample. One of the advantages of the methods of the invention is the ability to carry out any one or more of the steps of the methods (e.g., lysis, separation, interrogation, and/or identification) with the microorganisms in a sealed container (e.g., a hermetically sealed container). The present methods may involve the use of automated systems, thus avoiding the health and safety risks associated with handling of highly virulent microorganisms such as occurs with recovery of microorganisms from samples for direct testing.

The centrifugal concentrator may be any container with sufficient volume to hold the differential lysis buffer and a sample. In one embodiment, the container fits or can be fitted into a centrifuge rotor. Illustratively, the volume of the container can be about 0.1 ml to about 100 ml, e.g., about 50 ml. If the separation is done on a microscale, the volume of the container can be about 2 μl to about 100 μl, e.g., about 5 μl to about 50 μl. In one embodiment, the container has a wider internal diameter in an upper portion to hold the sample, and a narrower internal diameter in a lower portion where the pellet of microorganisms is collected. A tapered internal diameter portion can connect the upper and lower portions. Illustratively, the tapered portion can have an angle of about 20 to about 70 degrees, e.g., about 30 to about 60 degrees. In one embodiment, the lower narrow portion is less than half of the total height of the container, e.g., less than about 40%, 30%, 20%, or 10% of the total height of the container. The container can have a closure device attached or may be threaded to accept a closure device (e.g., a cap) such that the container can be sealed prior to centrifugation. In certain embodiments, the container is designed such that the microorganism pellet can be readily recovered from the container after separation, either manually or in an automated manner (so that technicians are not exposed to the container contents). For example, the container can comprise a removable portion or a break-away portion which contains the pellet and which can be separated from the rest of the container. In another embodiment, the container comprises one or more structures that permit access to the pellet after separation, such as one or more ports or permeable surfaces for insertion of a syringe or other sampling device or for drawing off the pellet. In one embodiment, the container is a stand-alone container, i.e., a device for separating a single sample. In other embodiments, the container is part of a device that comprises two or more centrifugal concentrators such that multiple samples can be separated at the same time. In one embodiment, the device comprises 2, 3, 4, 5, 6, 7, 8, 9, 10, 12, 15, 20, 25, 30, 36, 42, 48, 60, 72, 84, 96, or more centrifugal concentrators.

In another embodiment, the separation can be carried out by a filtration step in which the sample (e.g., a lysed sample) is placed in a device fitted with a selective filter or filter set with pore sizes that retain the microorganisms. Other examples of filtration include, but are not limited to, tangential flow filtration and/or buffer exchange separate the microorganisms from the sample, reduce the volume of sample, and concentrate the microorganisms. Suitable example of filtration techniques that may be used in the methods described herein are illustrated in FIGS. 15-20. The retained microorganisms may be washed by gently passing a suitable buffer through the filter. The washed microorganisms may then be interrogated directly on the filter and/or recovered for interrogation by directly sampling the surface of the filter or by back-flushing the filter with suitable aqueous buffer.

In one embodiment, the container can be a tube, e.g., a centrifuge tube. In another embodiment, the container can be a chip or a card. In one embodiment, the inventors have developed a centrifugal concentrator and associated apparatus, systems, and methods that can allow lysis of non-microorganism cells and recovery of microorganism cells to be carried out in a single tube. In addition, the microorganism pellet can be expressed from the centrifugal concentrator in such a way that the supernatant is isolated and contained within the upper portion of the centrifugal concentrator. Specifically, the centrifugal concentrator and associated apparatus, systems, and methods described herein enable a user to separate a microorganism from a sample in fewer operations with only a single centrifugation step. The centrifugal concentrator and associated apparatus, systems, and methods described herein also enable a user to separate and test the sample without handling the microorganism, thus avoiding the health and safety risks associated with handling of highly virulent microorganisms.

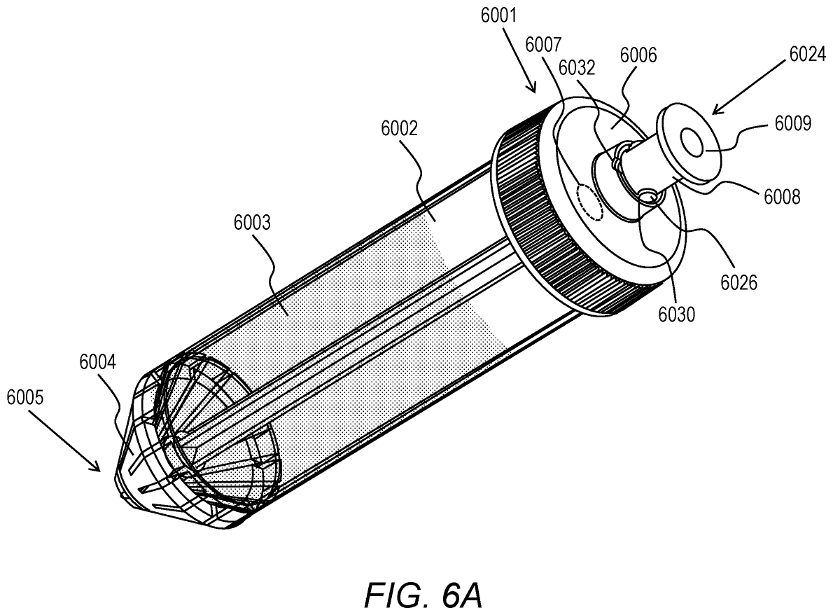

Referring to FIGS. 6A-6F, an embodiment of a centrifugal concentrator 5010 and elements of the centrifugal concentrator are illustrated. In one embodiment, the centrifugal concentrator is a centrifuge tube configured for concentration of microorganisms from a sample by centrifugation. Centrifugal concentrator 5010 includes a tube body 6002 and a closure cap 6006 at the proximal end 6001 of the tube body 6002. In one embodiment, a protective cap 6004 configured to protect the tube during centrifugation may be positioned on the distal end 6005 of the tube body 6002. In one embodiment, a differential lysis buffer and a sample (e.g., a whole blood sample) may be added to the tube body 6002 subsequent to removing cap 6006; and the contents may be sealed therein by replacing cap 6006. Illustratively, the distal 6005 and proximal 6001 ends of the centrifugal concentrator 5010 are sealed in operation to prevent the release of possibly biohazardous material, but, as will be explained in greater detail below, the distal end 6005 of tube body 6002 may be selectively openable to allow pelleted microorganisms to be ejected from the concentrator.

In the illustrated embodiment, centrifugal concentrator 5010 includes a plunger 6008. Plunger 6008 may be configured to perform a number of functions such as, but not limited to, gathering microorganisms that are concentrated (e.g., pelleted) at or near the distal end of the centrifugal concentrator 5010, piercing the distal end 6005 of the centrifugal concentrator 5010, and ejecting pelleted microorganisms from the distal end 6005 of the centrifugal concentrator 5010. The piercing of the distal end 6005 of the concentrator and the ejection of a microorganism pellet will be discussed in greater detail below in reference to FIGS. 6D and 6E. In one embodiment, plunger 6008 has a proximal end 6024 that includes a widened portion 6009 that is configured for manual manipulation of the plunger 6008. For instance, widened portion 6009 may be manipulated with a thumb, finger, or another part of a user's hand or with a mechanical device to actuate the plunger 6008 to eject a pellet. For example, the plunger 6008 may be depressed by the user's thumb to actuate the plunger and eject the pellet. In another embodiment, the plunger 6008 is actuated in a different manner, for example by rotating along a threaded screw portion to lower the plunger 6008 through the centrifugal concentrator. In the illustrated embodiment, plunger 6008 includes a pair of retaining members 6026 that are configured to keep the plunger in a locked ‘up’ position a first orientation and in a ‘plunge’ position in a second orientation. In one embodiment, retaining members 6026 interact with a corresponding pair of detents 6030 on the cap 6006 to hold the plunger in the locked ‘up’ position. In the illustrated embodiment, in order to plunge, the widened portion 6009 is grasped and used to twist the plunger relative to the cap so that the retaining members 6026 are aligned with passageway 6032. In one embodiment, passageway 6032 is configured to allow the plunger 6008 to be pushed down to eject a microorganism pellet from the distal end 6005 of the centrifugal concentrator.

In one embodiment, the centrifugal concentrator 5010 may have essentially any volume sufficient to hold the differential lysis buffer and a sample. In one embodiment, the centrifugal concentrator 5010 fits or can be fitted into a centrifuge rotor. Illustratively, the volume of the centrifugal concentrator 5010 can be about 0.1 ml to about 100 ml. In a specific embodiment, the centrifugal concentrator 5010 has a shape and interior volume similar to a standard 50 ml conical centrifuge tube and is compatible with centrifuge rotors (fixed angle and swinging bucket) designed to fit 50 ml conical centrifuge tubes. In one embodiment, a differential lysis buffer 6003 is provided in the centrifugal concentrator 5010. For example, approximately 20-40 ml (e.g., ˜30 ml) of the differential lysis buffer described herein may optionally be provided in the centrifugal concentrator 5010. While the centrifugal concentrator 5010 may include a differential lysis buffer 6003, illustratively the centrifugal concentrator may not be provided with a density cushion regardless of whether the differential lysis buffer is provided in the centrifugal concentrator 5010. In one embodiment, the cap 6006 of the centrifugal concentrator 5010 may optionally include a septum 6007 (e.g., a rubber septum) or a similar structure that may allow a sample (e.g., a whole blood sample) to be added to the centrifugal concentrator 5010 without having to remove the closure cap 6006. This may be particularly useful given that many of the samples intended to be used with the differential lysis buffer and the centrifugal concentrator may be biohazardous and/or infectious. In some embodiments, the sample is mixed with the differential lysis buffer and aseptically loaded into the centrifugal concentrator through the septum 6007. This reduces potential contamination of the sample prior to analysis.

FIG. 6B shows another view of the centrifugal concentrator 5010. In the view of FIG. 6B, the outer protective cap 6004 has been removed from the distal end of the tube body 6002 to show an inner support cap 6010 that caps a pellet collection reservoir (pellet collection reservoir 6014 in FIG. 6D). In addition to the outer protective cap 6004, the inner support cap 6010 may protect the distal end 6005 of the tube body 6002 to, for example, prevent leakage from the tube in storage or in use, particularly during centrifugation. In some embodiments, the support cap 6010 may be omitted. Removal of the distal protective cap 6004 also shows support ribs 6012 that may be included to reinforce and protect the distal end 6005 of the tube body 6002, particularly during centrifugation. The support ribs 6012 may be omitted in some embodiments. For instance, a specially designed centrifuge bucket insert may be configured to support the distal portion of the tube body 6002, possibly obviating the support ribs 6012.

FIG. 6C shows a view of the centrifugal concentrator 5010 similar to FIG. 6B, except the closure cap 6006 is removed to illustrate how the closure cap 6006 is attached to the tube body 6002. In the illustrated embodiment, the proximal end 6001 of the tube body 6002 includes threads 6011 that allow the closure cap 6006 to be threadably attached to the tube body 6002. Threads 6011 are merely illustrative, however. Threads 6011 could be replaced by any structure in the art known to perform the same or a similar function. For instance, closure cap 6006 could be sealed to the tube body 6002 by a bayonet mount, a friction arrangement, an o-ring assembly on the tube body 6002 or on the cap 6006, or the like.

Referring now to FIGS. 6D-6F, details of the distal end 6005 of the tube body 6002 and how the plunger 6008 may eject a microorganism pellet are illustrated. In the illustrated embodiment, the distal end 6005 of the tube body 6002 includes a pellet collection reservoir 6014. For reference, the pellet collection reservoir 6014 was shown covered by the support cap 6010 in FIGS. 6B and 6C. As discussed in greater detail elsewhere herein, in some embodiments centrifugal concentrator 5010 is configured to be centrifuged in a swinging bucket centrifuge such that the microorganisms are pelleted at the bottom of the tube (as opposed to on the sidewalls, as is typical with a fixed angle centrifuge rotor). Thus, substantially all of the unlysed microorganisms in the sample should be capable of being pelleted into the pellet collection reservoir 6014. In one embodiment, at least 75%, at least 80%, at least 85%, at least 90%, at least 95%, at least 99% or 100% of the microorganisms in the sample can be pelleted into the pellet collection reservoir 6014. In one embodiment, pellet collection reservoir 6014 may be sized and configured to contain substantially the entire microorganism pellet (e.g., less than or equal to ˜500 μl, ˜400 μl, ˜300 μl, ˜200 μl, 20-200 μl, 40-150 μl, or ˜50-100 μl). In some embodiments, the tube body 6002 may include sloped interior sidewalls 6015 (see FIGS. 6E and 6F) that are configured for funneling the microorganisms into the pellet collection reservoir 6014.

In some embodiments, the pellet collection reservoir 6014 may include a breakaway end 6016 that is configured to allow a portion of the plunger 6008 to be pushed through the pellet collection reservoir 6014 to eject a microorganism pellet. Suitable examples of a breakaway end include, but are not limited to, a thinner molded portion, a thinner molded portion with a frangible area, a foil cap, and the like.

Referring specifically to FIGS. 6E and 6F, details of how the plunger 6008 can pierce the pellet reservoir 6014 and eject a microorganism pellet from the distal end 6005 the tube body 6002 are shown. The plunger 6008 and the pellet reservoir 6014 are designed so that a microorganism pellet can be ejected from the pellet reservoir while isolating the spent lysate in the tube body. The plunger 6008 includes a distal portion 6030 with a tip 6022 that is configured for gathering the microorganism pellet and for piercing through the end 6016 of the pellet reservoir 6014 (e.g., through an affixed foil cap or through a frangible, breakaway portion). In the illustrated embodiment, the tip 6022 is shovel shaped with a sharp edge 6023 that can pierce through the end 6016 of the pellet reservoir 6014. While the tip 6022 is shown as shovel shaped in the illustrated embodiment, other suitable shapes include, but are not limited to, blade shapes, a blunt end, a spiked end, and the like. When the end of the pellet reservoir 6014 is pierced and the microorganism pellet is ejected, it is desirable that the spent isolate be left in the tube body so that the spent lysate does not leak and dilute the pellet. In some embodiments, the spent lysate may contain potentially infectious or biohazardous material, and for that reason containing the spent lysate is an important safety feature. Near the distal end 6030 of the plunger 6008, in one embodiment there is a portion 6018 that is sized and configured to mate with an inner portion 6020 of the pellet reservoir 6014. In this embodiment, the interface between portion 6018 of the plunger 6008 and inner portion 6020 of the pellet reservoir 6014 creates a seal that isolates the spent lysate in the tube body 6002. The interface may also ensure that the microorganism pellet is efficiently gathered and expelled. While the interface between 6018 and 6020 is shown as a friction fit, portion 6018 or portion 6020 may, for example, include an o-ring or a similar structure to create a seal that isolates the spent lysate in the tube body 6002. In some embodiments, actuating the plunger 6008 expels the pellet from the distal end of the centrifugal concentrator under pressure by opening the breakaway end 6016. In this embodiment, as the plunger is depressed the interface between portion 6018 and the inner portion 6020 of the pellet reservoir 6014 causes pressure to increase within the cavity until the breakaway end 6016 is pierced, resulting in the microorganism pellet being expelled from the pellet reservoir 6014. This may cause greater recovery of the microorganism because the pressure reduces the chance that the microorganism will be retained on the sides of the pellet reservoir.

Referring now to FIG. 6G, the plunger 6008 is shown on its own. Plunger 6008 includes the proximal end 6024, distal end 6030, retaining members 6026, portion 6018, and shovel tip 6022 discussed elsewhere herein. The plunger 6008 includes a plunger shaft 6028 that, in one embodiment, is sized to be long enough (e.g., substantially the same length as the tube body 6002) so that when the plunger is plunged it can pierce the end of the tube body 6002 and eject the microorganism pellet. In addition, the shaft may optionally include an o-ring 6027 or a similar structure that may be configured to mate with the cap 6006 to seal the interface between the cap and the plunger. Of course, the cap may also include a sealing member in lieu or in addition to the o-ring 6027. It is understood that centrifugal concentrator 5010 is illustrative only. Centrifugal concentrator 5010 and its variations discussed above as well as other vessels may be used with the various methods disclosed herein.

Interrogation Step

Once the microorganisms have been pelleted, the pellet can be interrogated to identify and/or characterize the microorganisms in the pellet. In one embodiment, the interrogation may take place in a non-invasive manner, that is, the pellet is interrogated while it remains in the centrifugal concentrator. The ability to identify the microorganisms in a non-invasive manner, optionally coupled with keeping the container sealed throughout the separation and identification process and automating some or all of the procedure avoids the constant handling of contaminated and/or infectious samples and greatly increases the safety of the entire process.

In another embodiment, the pellet can be interrogated using molecular techniques (e.g., PCR) to amplify sequences of microorganism RNA or DNA that can be used to individually identify each of the types of microorganisms that may be in the sample. In one example, nucleic acid sequences may be selected that are characteristic for each of the individual types of bacteria, fungi, and the like that may be in the sample, forward and reverse primers may be designed for amplification of those sequences, microorganisms in the pellet may be lysed, and the lysate (or purified nucleic acid from the lysate) may be combined with the primers and other PCR reagents (buffer, polymerase, etc.) so that the selected, characteristic nucleic acid sequences may be amplified according to well-known procedures in the art. Amplified nucleic acids may be detected and used to identify the presence of one or more microorganisms in the sample according to well-known procedures in the art, such as, but not limited to, real-time detection or post-amplification analysis such as melting-curve analysis, other dsDNA binding dye techniques and probes that are labeled fluorescently, radioactively, chemiluminescently, enzymatically, or the like, as are known in the art. In one embodiment, the pellet can be interrogated using the FilmArray system described in detail elsewhere herein. In one embodiment, the pellet can be interrogated using a specially adapted Blood Culture Identification (BCID) panel pouch and protocol. The BCID panel and protocol are described in U.S. Pat. No. 10,053,726, the entirety of which is incorporated herein by reference. However, the BCID is merely one example of an assay device. Aperson of ordinary skill will understand that the pellet may suitably be interrogated using a specially designed assay that has, for example, sensitivity and limit of detection values suitably adapted to the concentration of organisms in a sample obtained direct from blood.

In another embodiment, the pellet can be interrogated by sequencing the nucleic acids present in the pellet. Sequencing characteristic microorganism sequences or whole microorganism genomes can be used to identify the microorganisms in the pellet. Such sequencing may be performed according to one or more of the many sequencing techniques known in the art. In one embodiment, the sequencing is Sanger sequencing. In another, preferred embodiment, the sequencing includes a massively parallel or “Next Generation” Sequencing (NGS) technique. Massively parallel/NGS technologies process hundreds of thousands to millions of DNA fragments in parallel, resulting in a low cost per base of generated sequence and a throughput on the gigabase (Gb) to terabase (Tb) scale in a single sequencing run. As a consequence, massively parallel/NGS techniques can be used to define the characteristics of entire genomes at low cost and with high throughput.

### Example 1—Detection of Microorganisms Direct from Whole Blood

The differential lysis buffer and centrifugal concentrator described herein can be used for detection of microorganisms direct from whole blood. This can be used, for example, to rapidly identify sepsis-causing microorganisms without the step of pre-culturing a blood sample to amplify disease causing organisms prior to detection. As discussed elsewhere herein, however, such detection and diagnosis direct from whole blood has proved to be difficult for a number of reasons. For one, the number of infectious organisms found in whole blood in BSI is usually low (˜1-100 colony-forming units per milliliter of blood (cfu/ml) with ˜1-10 cfu/ml being typical in most individuals with culture-confirmed sepsis), and blood contains a number of inhibitors of the Polymerase Chain Reaction (PCR) (e.g., hemoglobin and genomic DNA from white blood cells that can co-purify with microorganisms and interfere with both nucleic acid recovery from the target microorganisms and downstream PCR).

With so few organisms in whole blood and the presence of PCR inhibitors, concentrating from larger volumes of whole blood (e.g., 1-20 mL) is desired to obtain the quality and quantity of DNA template desired to achieve sensitivity at clinically relevant microorganism levels. In one embodiment, the differential lysis buffer and centrifugal concentrator described herein allow technicians to lyse non-microorganism cells in about 1-20 mL (e.g., about 10 ml) of whole blood and concentrate the microorganisms therein by centrifugation in about 5-20 minutes (e.g., about 15 minutes). Illustratively, sample lysis does not involve a DNase step and the centrifugal concentrator does not use a density cushion. This makes sample preparation more rapid, easier, and more reproducible.

The microorganism pellet obtained from the centrifugal concentrator described herein can be ejected directly into a sample vial that can be used to injected the sample into a molecular assay device. In a specific example, the microorganism pellet obtained from the centrifugal concentrator can be ejected directly into a FilmArray injection vial (FAIV) (described in U.S. Pat. No. 10,464,060, the entirety of which is incorporated herein by reference) and then into a FilmArray pouch. In one embodiment, the FAIV can be used to inject the microorganisms obtained from the centrifugal concentrator directly into a Blood Culture Identification (BCID) panel pouch and for identification of the microorganism in the sample. Currently, the BCID panel assay takes about 60 minutes to perform. The BCID panel assay and protocol are described in U.S. Pat. No. 10,053,726, the entirety of which was incorporated hereinabove. Using the differential lysis buffer and the centrifugal concentrator in concert with the BCID panel assay and a specially modified instrument protocol, the inventors in this case have achieved a limit of detection of about 1-10 cfu/ml and a sample preparation and analysis time of about 75 minutes in total. However, it is likely that the analysis time can be reduced significantly as chemistry and instrument performance are further improved.

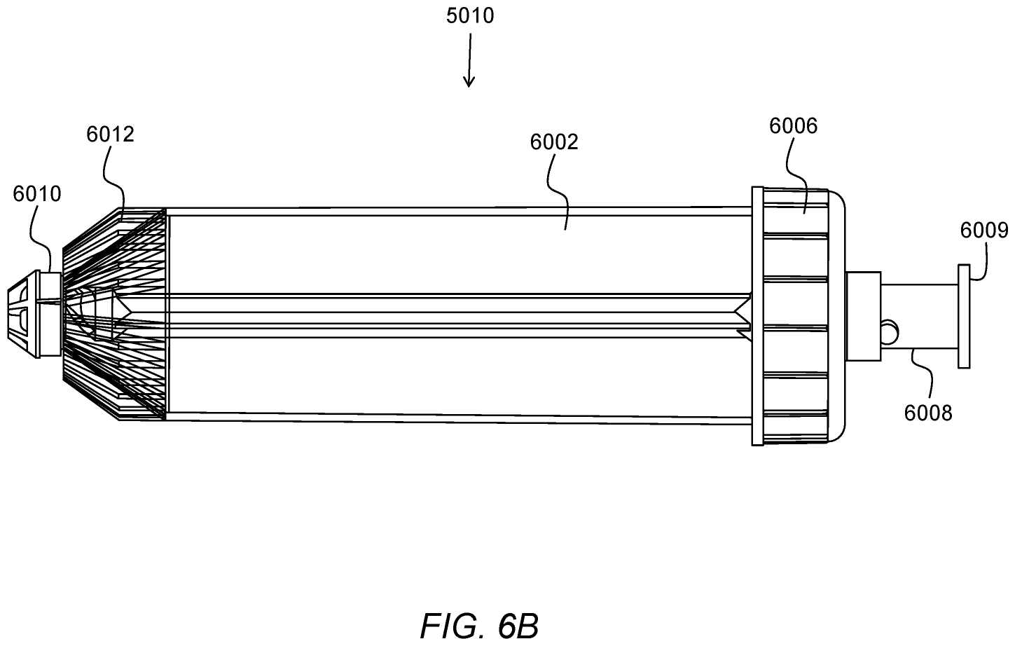

Referring now to FIG. 7, an example of a sample preparation workflow is illustrated. In a first step 700, a blood sample and a centrifugal concentrator are provided. In one example, the blood sample, which may have a volume of about 10 ml, is provided in a standard vacutainer. In one example, the centrifugal concentrator may be provided with a volume of differential lysis buffer (e.g., about 30 ml) therein.

In a second step 702, the blood sample and the differential lysis buffer of Table 1 were combined for a period of time sufficient to lyse substantially all of the non-microorganism cells (i.e., all of the blood cells) in the sample to yield a lysate. For example, the blood sample and the differential lysis buffer may be combined for about 1-5 minutes, although longer or shorter times may be used. Illustratively, the lysis can take place at a temperature of about 2° C. to about 45° C., e.g., about 15° C. to about 40° C., e.g., about 30° C. to about 40° C. In one embodiment, the lysis may take place at room temperature.

Subsequent to combining the the blood sample with the differential lysis buffer for a sufficient time to yield a lysate, the microorganisms, if present, may be recovered from the lysate by centrifugation in step 704. In one embodiment of the invention, the centrifugal concentrator may be centrifuged in a swinging bucket rotor so that the microorganisms form a pellet directly on the bottom of the tube. The container is centrifuged at a sufficient acceleration and for a sufficient time for the microorganisms to pellet and/or be separated from other components of the sample. In one embodiment, the centrifugation time can be about 30 seconds to about 30 minutes, e.g., about 10-15 minutes. Illustratively, the centrifugal acceleration can be about 1,000×g to about 20,000×g, e.g., about 3000-10,000×g. Illustratively, the centrifugation can be carried out at a temperature of about 2° C. to about 45° C., e.g., about 4-8° C.

If the centrifugal concentrator includes an optional distal support cap, the support cap can may be removed prior to plunging, as illustrated in step 706. Steps 708-712 illustrate an example process for ejecting a microorganism pellet from the centrifugal concentrator. In step 708, at the beginning of the plunge, the distal end of the plunger can move into the pellet reservoir and isolate the pellet from the supernatant. This was illustrated in FIG. 6E, which was discussed herein above. As the plunger is pushed further into the pellet reservoir, the breakaway end of the pellet reservoir can be punctured, separated, fractured, or the like and the pellet can begin to be ejected, as illustrated in step 710. Step 712 in the workflow shows the pellet being fully ejected from the centrifugal concentrator. The ejected pellet can be used in a number of assays know in the art for characterizing and identifying the microorganisms in the pellet. As discussed herein above, PCR analysis and sequencing are two non-limiting examples of assays that can be used for characterizing and identifying the microorganisms in the pellet.

As illustrated at 714, in one embodiment, the distal end of the centrifugal concentrator may be sized and configured to fit directly into a receptacle. In one embodiment, the receptacle is a FilmArray Injection Vial (FAIV). As such, the pellet can be ejected directly into the FAIV; the FAIV can then be used to inject the sample into a FilmArray assay pouch for characterization and identification of the microorganisms in the pellet. In one embodiment, the pellet may be ejected directly into the FAIV and the FAIV may be used to inject the microorganisms into a FilmArray pouch without detaching the FAIV from the centrifugal concentrator. After using the FAIV to load the sample into a FilmArray pouch, the whole assembly may be disposed of in a biohazard waste container. This reduces the handling of potentially infectious organisms and potentially biohazardous waste and limits the risk of contamination.

In one embodiment, an aliquot (e.g., approx. 1 ml) of the original sample may be added to the pellet ejected in step 712, to the vial of step 714 along with the pellet, or directly to an analysis device along with the pellet. The lysis and centrifugation methods (and related methods) described herein are well suited to the concentration and detection of microorganisms like bacteria and yeast, but they are not particularly suited for the detection of viruses. By adding an aliquot of the original sample to the pellet and to the analysis, viruses may also be isolated and detected.

In one aspect of the invention, some or all of the method steps can be automated. Automating the steps of the methods allows a greater number of samples to be tested more efficiently and reduces the risks of human errors in handling samples that may contain harmful and/or infectious microorganisms. Of greater importance, however, automation can deliver critical results at any time of the day or night without delay. Several studies have shown that faster identification of the organisms causing sepsis correlates with improved patient care, shorter hospital stays and lower overall costs.

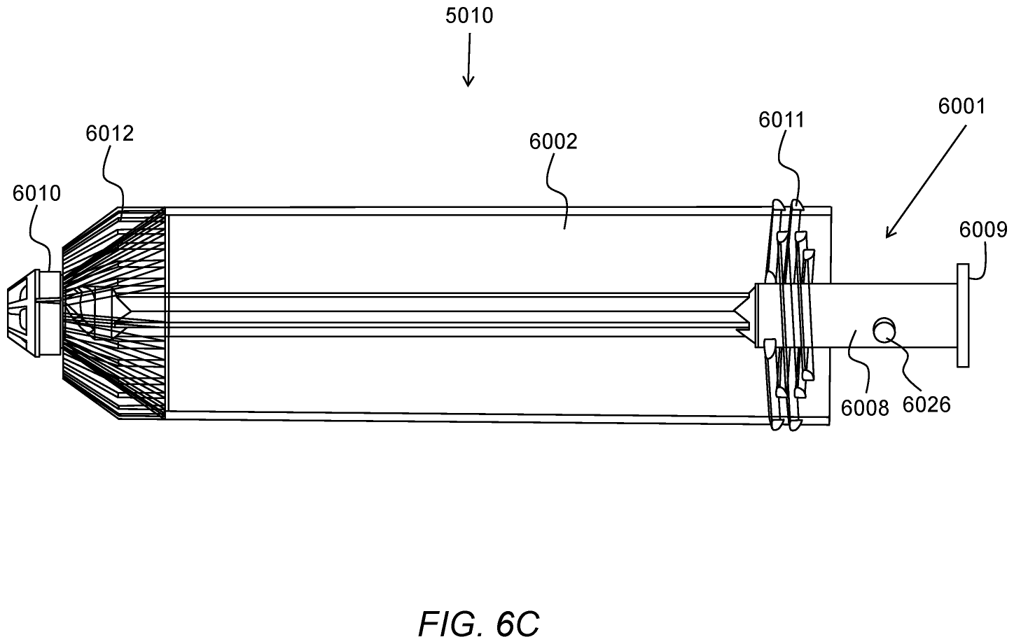

Referring now to FIG. 8, the sample preparation time for the methods described herein using the differential lysis buffer and centrifugation are compared to other procedures. As can be seen from FIG. 8, the method of combining the sample with the differential lysis buffer and subsequent centrifugation method involves only two steps and a total of about 15 minutes of processing time. This is significantly faster and easier than the other procedures examined by the inventors in this case. The MolYsis procedure discussed in the Introduction section of this document involves a number of complicated steps—including a DNase step—and takes about 45-50 minutes just for eukaryotic cell lysis, DNase treatment, and microorganism cell recovery. Microorganism cell washes and lysis require additional steps and buffers. Successful use of the MolYsis system requires a skilled technician. The dependence on skill of the operator raises the risk of operator-to-operator differences in yield and quality of results. The many buffers and manual pipetting steps increases the risk of cross-contamination of samples. The steps of combining the sample with the differential lysis buffer and subsequent centrifugation does not require many of those steps, including a DNase step nor many of the wash steps. In the methods described herein, the microorganism cells recovered after the centrifugation are suitable for molecular analysis (e.g., a PCR assay, DNA sequencing, or mass spectrometry) after centrifugation without further treatment.

FIG. 8 also compares the sample preparation time of the differential lysis and centrifugation method with two other protocols. The Y2 Protocol is a procedure that uses a saponin-based lysis combined with protease and DNase digestion steps. The Y2 Protocol required a number of complicated steps and ˜90 minutes for sample preparation. The Lycoll+DNase protocol is a procedure that uses a saponin-based lysis combined with DNase digestion and a ficoll gradient for centrifugation. The Lycoll procedure produced good organism yields and effectively removed genomic DNA, but it involved complicated layering of the lysate on the ficoll gradient and an approximately 2 hour processing time. As compared to the methods claimed herein using the differential lysis buffer and subsequent centrifugation, the Y2 Protocol and the Lycoll+DNase procedure both involve complicated steps and too much time. The same is true for the comparison of the differential lysis and centrifugation method with the MolYsis procedure.

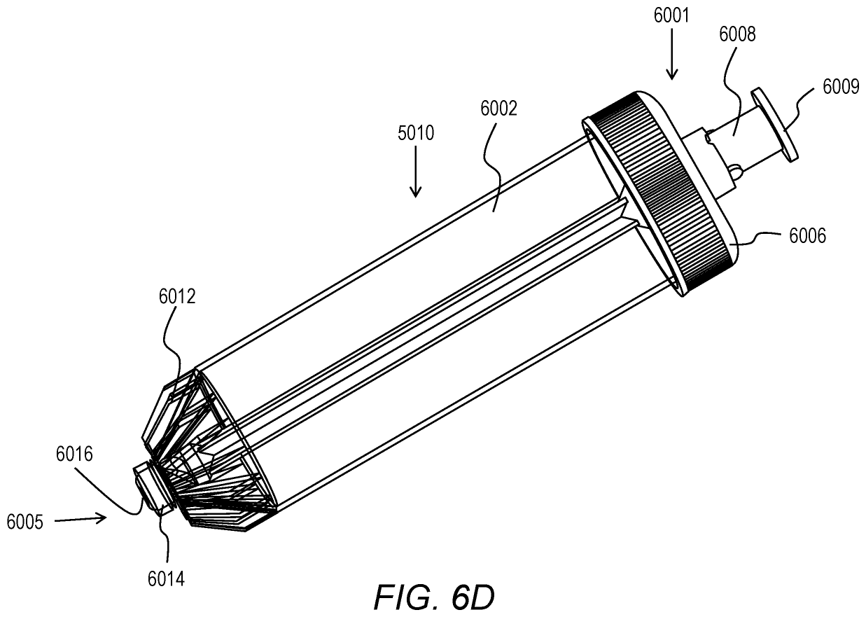

FIG. 9 illustrates the microorganism recovery in one set of experiments that can be achieved by treating a sample with the differential lysis buffer and subsequent centrifugation. The control is a spiked buffer and the test samples are spiked whole blood. The control and the test samples were spiked with the same numbers of organisms. The control tests the ability to recover the spiked organisms from buffer by centrifugation while the test samples demonstrate the efficacy of the differential lysis buffer for lysis of eukaryotic cells (i.e., RBCs, white blood cells, platelets, etc.) and recovery of the spiked microorganism cells from the lysate by centrifugation. As compared to the control, in this experiment, the method that includes treating the spiked blood sample with the differential lysis buffer and subsequent centrifugation can recover about 86% of microorganisms from a whole blood sample. As illustrated in Table 2, the recovery rate can be over 90% in other experiments.

TABLE 2

| | 50 mL | Centrifugal | Centrifugal |
|---|---|---|---|
| | conical | concentrator | concentrator |
| Condition | Tube | (w/ plunger) | (w/o plunger) |
| Organism | 191 | 231 | 222 |
| Count | | | |
| % Recovery | 76% | 92% | 89% |

In a preferred embodiment, the recovery of the microorganisms with differential lysis and subsequent centrifugation can be at least 85%, at least 90%, at least 95%, at least 99% or 100%.

Treating a blood sample with the differential lysis buffer and subsequent centrifugation also efficiently removes genomic DNA, all without including a DNase step or other complicated or time-consuming processing steps. Table 3 below compares the degree of genomic DNA removal from whole blood achieved with the differential lysis buffer and centrifugation to the Lycoll and Lycoll+DNase methods. The Whole Blood Control represents the amount of genomic DNA recovered from a lysed whole blood sample. DNA was purified with the MagnaPure system and quantified with the ThermoFisher Quantifiler Human DNA Quantification Kit.

TABLE 3

| Condition | Average μg DNA in Pellet | % in Pellet |
|---|---|---|
| Lycoll | 10.1 | 5% |
| Lycoll + Dnase | 2.1 | 1% |
| Differential lysis buffer | 3.7 | 2% |
| Whole Blood Control | 213.6 | 100% |

As can be seen from Table 3, the amount of genomic DNA recovered with differential lysis buffer and centrifugation treatment is significantly better than the Lycoll method and is comparable to the Lycoll+DNase method. Differential lysis buffer and centrifugation treatment is significantly faster, easier, and more reproducible than the Lycoll or Lycoll+DNase methods, and differential lysis buffer and centrifugation treatment achieves an impressive genomic DNA reduction without a time-consuming DNase step.

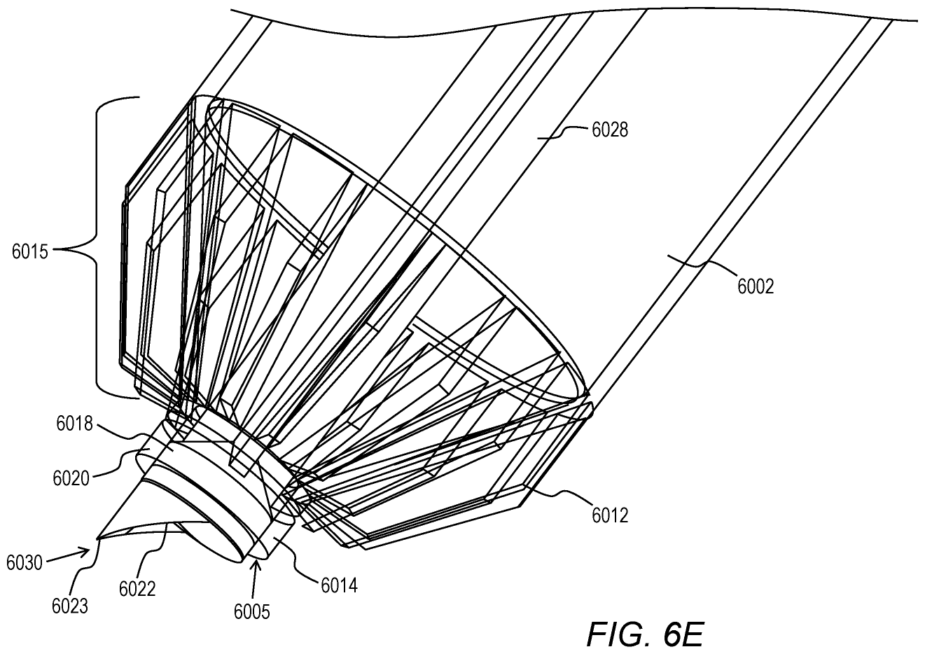

This is demonstrated slightly differently in FIG. 10, which compares crossing-point (Cp) values for amplification of varying amounts of a yeast control in the presence of whole blood material that can be pelleted by centrifugation after treatment of the blood with the differential lysis buffer. In this case, the differential lysis buffer (• Alkaline) was compared to the Lycoll method (* Lycoll). For the differential lysis buffer experiments, 10 mL whole blood was spiked with 1, 10, or 100 CFU/mL of a yeast control and then processed using the differential lysis buffer and subsequent centrifugation, as described herein. The resulting pellets were transferred into a FAIV and the samples were run on FilmArray BCID pouches. The Lycol experiments were run similarly, except the 10 ml of spiked whole blood was processed using the Lycoll method. The CFU Control (▪) represents the Cp for the varying amounts of yeast DNA in the absence of any whole blood material. Different CFU amounts of yeast were diluted in PBS and pipetted into a FAIV at levels equivalent to a 100% concentration of organism from 10 mL of spiked whole blood used for the differential lysis buffer and Lycoll procedures (1 CFU/mL in whole blood=10 CFU Control into FAIV). The WB control (□) is unconcentrated whole blood. For the WB control, 200 μL of spiked whole blood was pipetted into a FAIV at levels equivalent of a 100% concentration of organism from 10 mL of spiked whole blood used for the differential lysis buffer and Lycoll procedures to show the initial LoD/Cp values of organism prior to concentration protocols. As can be seen in FIG. 10, the amplification of yeast DNA in the presence of the Lycoll pellet was delayed by ˜3 Cp units as compared to CFU and WB controls. In contrast, there was no detectable inhibition from the pellets obtained using the differential lysis buffer and subsequent centrifugation. I.e., amplification of the yeast DNA in the presence of the pellets obtained from the differential lysis buffer is virtually indistinguishable from the CFU and WB controls. It appears that the increased Cps in the Lycoll pellet are due to high levels hgDNA being concentrated in the pellet along with the spiked yeast organisms. hgDNA is a known competitive inhibitor for DNA recovery with magnetic silica beads and a non-specific inhibitor of PCR. Based on the data shown in Table 3 and based on these data, it is concluded that the pellets obtained from the differential lysis buffer do not have as much hgDNA in the pellet and, as a result, has yeast DNA Cps more similar to that of the whole blood control or even the CFU control which does not have any matrix.

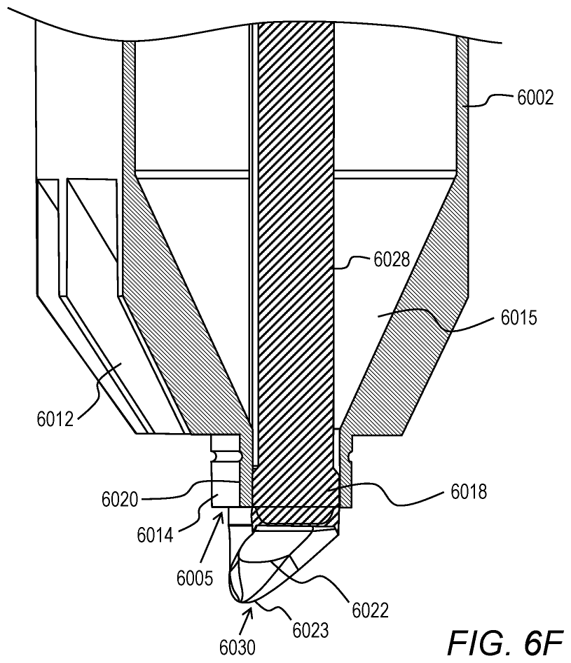

Referring now to FIG. 11, data are presented for different differential lysis buffer formulations (LB18-LB21) with varying amounts of CAPS and Brij O10. For the lysis buffer tests, 10 ml samples of whole blood were spiked with E. coli, enteric bacteria, or yeast and processed using the designated differential lysis buffer and centrifugation treatment, according to the methods described herein. The resulting pellets were transferred into a FAIV and the samples were run on FilmArray BCID pouches, as described for FIG. 10. The WB control is the same as was described for FIG. 10. The data presented in FIG. 11 show that the differential lysis buffer efficiently lyses host cells (i.e., RBCs, white blood cells, platelets, etc.) and host cell nuclei while leaving microorganism cells intact and pelletable by centrifugation.

With the differential lysis buffer disclosed herein, sample processing involves only two simple steps and processing time may be reduced to ˜15 minutes. The DNase step that is common in other methods may be eliminated due to effective rupture of the nuclear membrane. The volume of the pellet obtained with the differential lysis buffer and subsequent centrifugation may suitably be <200 μL. Brij O10 and CAPS completely lysed blood cells within seconds or minutes. As demonstrated in Example 1, this buffer is easy to use and therefore results should be more reproducible (FIG. 8). Microorganism cells can be concentrated from whole blood rapidly (FIG. 8), a high proportion of microorganism cells in the sample can be recovered (FIG. 9 and Table 2), human genomic DNA can be reduced from the microorganism pellet (FIG. 10 and Table 3), and host cells and host cell nuclei can be effectively lysed while leaving microorganism cells intact and recoverable by centrifugation (FIG. 11).

### Example 2—Microbial Recovery by Species at Low Spiking Level (<1 CFU/mL)

In the previous Example, it was demonstrated that the differential lysis buffer and centrifugal concentrator described herein can be used for lysis of whole blood factors, recovery of microbial cells, and then detection of the microorganisms. This Example expands on Example 1 and demonstrates the capability to recover and identify microorganisms at low levels (i.e., <1 CFU/mL) from spiked whole blood. For most cases of blood stream infections (i.e., sepsis), clinically relevant microorganism levels in whole blood range from about <1 CFU/mL up to about 10 CFU/mL. This Example also demonstrates the capability to recover and identify microorganisms at clinically relevant levels on a species-by-species basis.

The direct from blood processing method described herein is an uncomplicated workflow with steps that lyse, centrifuge, and eject the pellet to recover organisms from the lysate. In this Example, the whole blood sample was mixed with the differential lysis buffer and lysis was allowed to proceed for about 5 minutes and the lysate was centrifuged for about 30 min at about 3000×g to recover the microorganisms. The lysis buffer used in this example is shown in Table 4

TABLE 4

| | Pre | | | | Post | | | |
|---|---|---|---|---|---|---|---|---|
| | Brij | CAPS | | | Brij | CAPS | | |
| | 010 | (mMol) | NaCl | pH | 010 | (mMol) | NaCl | pH |
| LB100 | 0.25% | 150 | 0.90% | 10.20 | 0.167% | 100 | 0.60% | 9.80-9.95 |

The comprehensive test panel included 120 organism strains from 12 species of bacteria and yeast most commonly isolated from blood stream infections. The harvested organisms not only maintain viability but have a reduced level of blood debris and contaminating host DNA, facilitating potential use as input into various downstream applications, both growth-based and molecular.

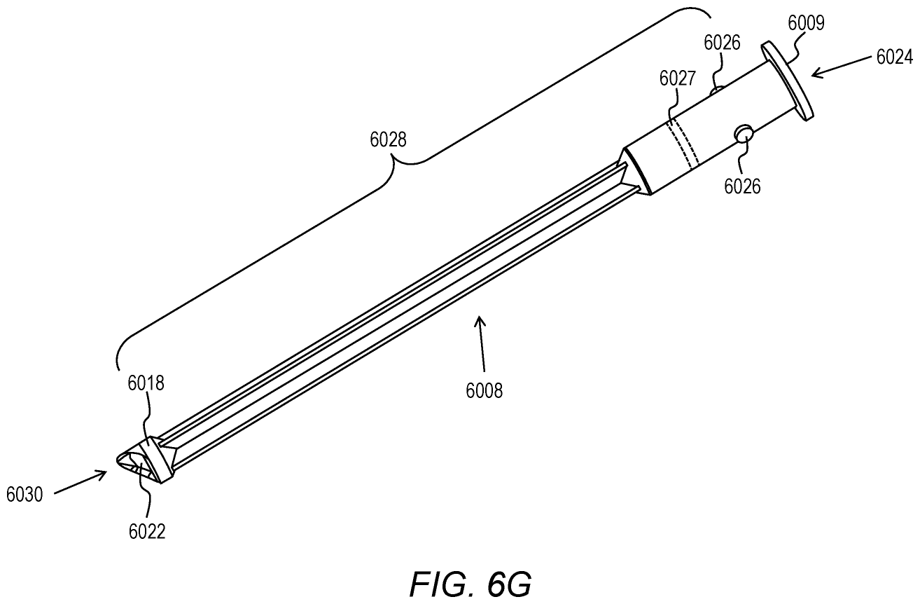

FIG. 12 illustrates the workflow used in this study. In steps (a) and (b), an organism stock of the appropriate concentration (e.g., about 100 CFU/ml) may be obtained by serially diluting and plating an organism stock until the desired concentration is achieved. The concentration may be verified by plating the stock solution onto agar plates and growing individual colonies on the plates. For example, plating 50 μL of a 100 CFU/ml stock should yield 5 colonies/plate. The stock organism solution may be diluted and plated several times in order to achieve the desired concentration. In step (c), the stock organism solution (e.g., ˜100 CFU/mL) was spiked into whole blood. In the example illustrated in FIG. 12, 150 μL of organism stock was spiked into 30 mL of whole blood. It was desired that the organisms be spiked in at a concentration of <1 CFU/mL. In the example shown in FIG. 12 the target spiking concentration was 0.5 CFU/mL. As will be explained in greater detail below, the spiking varied between Gram negative, Gram positive, and yeast organisms. The spiked whole blood was divided into three 10 mL fractions and combined in a centrifugal concentrator with 20 mL of LB100 buffer and allowed to lyse at room temperature 5 min. Steps (d)-(f). In the specific example illustrated in FIG. 12, the blood and lysis buffer were inverted in the centrifugal concentrator tube 10 times, incubated at RT for 5 min., vortexed for about 5 seconds, and then centrifuged for 30 min. at 3000×g in a swinging bucket centrifuge rotor.

Following centrifugation, the pellet from the centrifugal concentrator was ejected into into 500 μL TSB (tryptic soy broth) (step (g)) and 100 μL was plated onto each of five agar plates (step (h)). The plates were incubated for 24 hrs at 37° C. (step (i)) and the CFU from the five plates were added to obtain the total recovery (step (j)). While plating and culturing are used for detection of organisms in this Example, the workflow could be used for a variety of different types of detection. For example, molecular detection techniques such as, but not limited to, PCR (e.g., with the FilmArray system, as discussed in detail herein), whole genome sequencing, or molecular AST could be used. Phenotypic (e.g., Vitek2 AST), proteomic (e.g., maldi-TOF, Vitek MS, etc.), and microscopic techniques may be used to interrogate the pellet obtained from the centrifugal concentrator.

Results of this research study are summarized below:

Percent recovery for all organisms, Gram negative, Gram positive, and yeast are shown below in Table 5. The average overall recovery for this study was 80%, which exceeded the target goal of >70%.

TABLE 5

| | Overall | Gram Negative | Gram Positive | Yeast |
|---|---|---|---|---|
| Avg % Recovery | 80 | 78 | 83 | 77 |

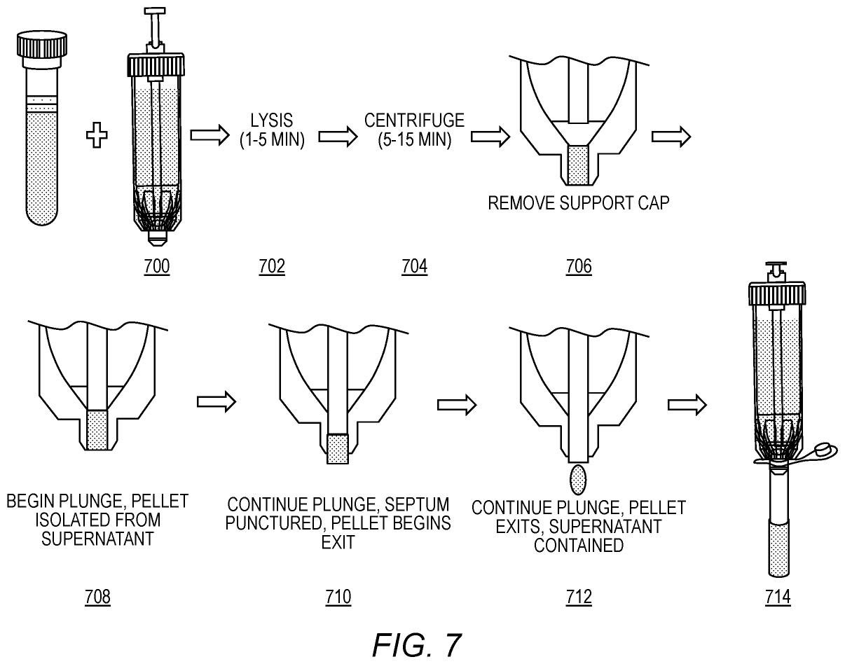

FIG. 13 illustrates that there was some variability among recovery rates by organisms, but all organisms could be recovered and cultured. As can be seen in FIG. 13, recovery of S. pneumoniae strains was poor in this study. Recovery rates for Gram positive species rise to 95% when S. pneumoniae strains are excluded, with overall recovery rising to 84%. Further method optimization, e.g., pH, contact time, supplementation, may improve recovery of this species. It is also possible that S. pneumoniae strains are less viable after recovery from the lysis buffer (as compared to other organisms) and that their apparent low relative recovery would rise if a detection technique that did not rely on viable organisms (e.g., a molecular technique) was used for detection.

Percent recovery by CFU for all organisms, Gram negative, Gram positive, and yeast are shown below in Table 6.

TABLE 6

| | Avg CFU | | | | |
|---|---|---|---|---|---|
| Sample | | Overall | Gram Negative | Gram Positive | Yeast |
| Inoculum | Input | 9.0 | 9.8 | 6.8 | 12.5 |
| | Output | 6.9 | 7.3 | 5.2 | 9.9 |

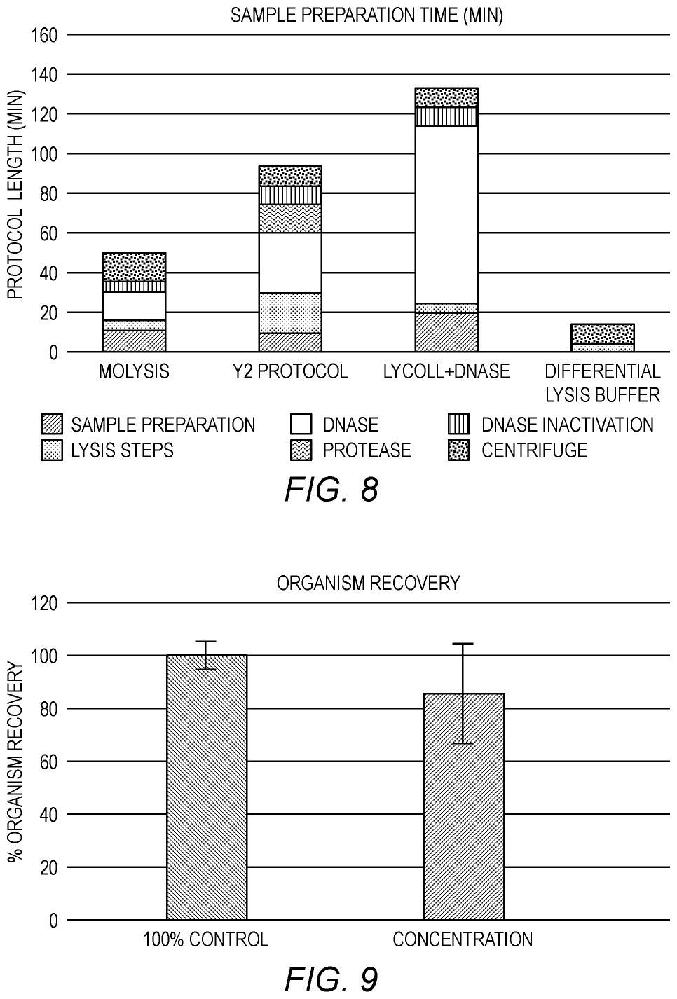

The measured input inoculum levels for all organisms were slightly higher than target of 5 CFU/10 mL. Nevertheless, the overall the goal of achieving recovery and detection of <1 CFU/mL from whole blood was met. FIG. 14 breaks out the data of Table 6 on an organism-by-organism basis;

Table 7 (below) shows the reduction in contaminating host DNA.

TABLE 7

| | Average | Min | Max |
|---|---|---|---|
| Lysate Input - Total μg/30 ml | 454 | 285 | 645 |
| output-Total μg/0.5 ml | 1.6 | 0.4 | 3.6 |
| % Reduction | 99.7 | 99.4 | 99.9 |

An average 99.7% decrease in host DNA was calculated from the input DNA concentration of 10 mL blood in lysate to the host DNA in the output pellet. The range shown reflects variation over 15 different blood donors and test days.

For a comprehensive panel of organisms, this study demonstrated recovery at low spiking level (i.e., <1 CFU/mL) from whole blood. The recovery and detection sensitivity in this study are comparable to the sensitivity of traditional blood culture (4-8 CFU/10 mL). The CAPS-Brij lysis buffer lyses and solubilizes human blood cell membranes, RBCs and WBCs. The CAPS-Brij lysis buffer also reduced blood cell debris and DNA in the output pellet. The processing method concentrates and collects viable organisms with a significantly reduced level of blood debris and host DNA, providing a potentially suitable sample for multiple rapid diagnostic pathways.

### Example 3—Culturing Microbial Cells after Whole Blood Lysis and Recovery

In this Example, different differential lysis buffer compositions were compared for detection within a FilmArray BCID assay pouch. Only three organisms were used in this comparison: C. albicans, E. coli, and S. agalactiae. ACD (anticoagulant citrate dextrose) anticoagulant blood was used in this study. This study demonstrates (1) that the differential lysis buffer/centrifugation procedure described in this application can enrich cells with all buffer compositions and organisms tested and (2) post-centrifugation culture was capable of enriching cells for organisms isolated from blood and lysis buffer for all selective lysis buffer compositions tested. All selective lysis buffers tested are capable of lysing blood cells while leaving microbial cells intact and viable.

The buffers tested are listed below in Table 8.

TABLE 8

| | Pre | | | | Post | | | |
|---|---|---|---|---|---|---|---|---|
| | Brij | CAPS | | | Brij | CAPS | | |
| | 010 | (mMol) | NaCl | pH | 010 | (mMol) | NaCl | pH |
| LB20 | 0.33% | 13.33 | 0.42% | 10.65 | 0.25% | 10.00 | 0.32% | 7.60 |
| LB19 | 0.33% | 33.33 | 0.41% | 10.49 | 0.25% | 25.00 | 0.30% | 8.15 |
| LB16 | 0.33% | 66.67 | 0.38% | 10.50 | 0.25% | 50.00 | 0.28% | 9.59 |
| LB100 | 0.33% | 133.33 | 0.62% | 10.53 | 0.25% | 100.00 | 0.42% | 10.30 |

LB20 is the buffer listed in Table 1 is the buffer that was used in the study described in Example 2.

The data in Table 9 demonstrates the improvement in crossing point (Cp) of the alkaline lysis/centrifugation method relative to unconcentrated, spiked whole blood. The Cp improvements are likely due to removal of substances that interfere with PCR (e.g., hemoglobin) and due to concentration of cells in the sample.

TABLE 9

| | | | | Average |
|---|---|---|---|---|
| Condition | Brij O10 | CAPS (mMol) | NaCl | ΔCp = WB − X |
| LB16 | 0.33% | 66.67 | 0.38% | 3.03 |
| LB19 | 0.33% | 33.33 | 0.41% | 2.43 |
| LB20 | 0.33% | 13.33 | 0.42% | 2.27 |
| LB100 | 0.33% | 150 | 0.62% | 1.34 |

For buffers LB16, LB19, and LB20, the apparent enrichment was about 8-fold and the apparent enrichment for LB100 was about 2.5-fold. One cycle of Cp improvement represents about a 2-fold increase in input concentration of target cells or template DNA, a two cycle Cp improvement represents about a 4-fold increase, a three cycle Cp improvement represents about a 8-fold increase, etc. (by the general formula, an n cycle Cp improvement represents about a 2ⁿ-fold increase in input concentration of target cells or template DNA).

The data in Table 10 demonstrates the improvement in Cp that results in culturing the pellet collected from the centrifugal concentrator in media for 3 hrs. 150 uL of BHI broth was mixed with the pellet from the centrifugal concentrator and incubated at 37° C. for 0 hr or 3 hr. The Cp improvement shown in Table 10 represents the average reduction in Cp (i.e., shorter time to detection) observed for the 3 hr culture relative to the sample cultured for 0 hr.

TABLE 10

| | | | | Average |
|---|---|---|---|---|
| Condition | Brij O10 | CAPS (mMol) | NaCl | ΔCp = 0 hr − 3 hr |
| LB16 | 0.33% | 66.67 | 0.38% | 1.15 |
| LB19 | 0.33% | 33.33 | 0.41% | 3.53 |
| LB20 | 0.33% | 13.33 | 0.42% | 3.54 |
| LB100 | 0.33% | 150 | 0.62% | 3.01 |

Culturing the cells for 3 hr enriched the cells from LB16 by about 2-fold, about 12-fold for LB19 and LB20, and about 8-fold for LB100.

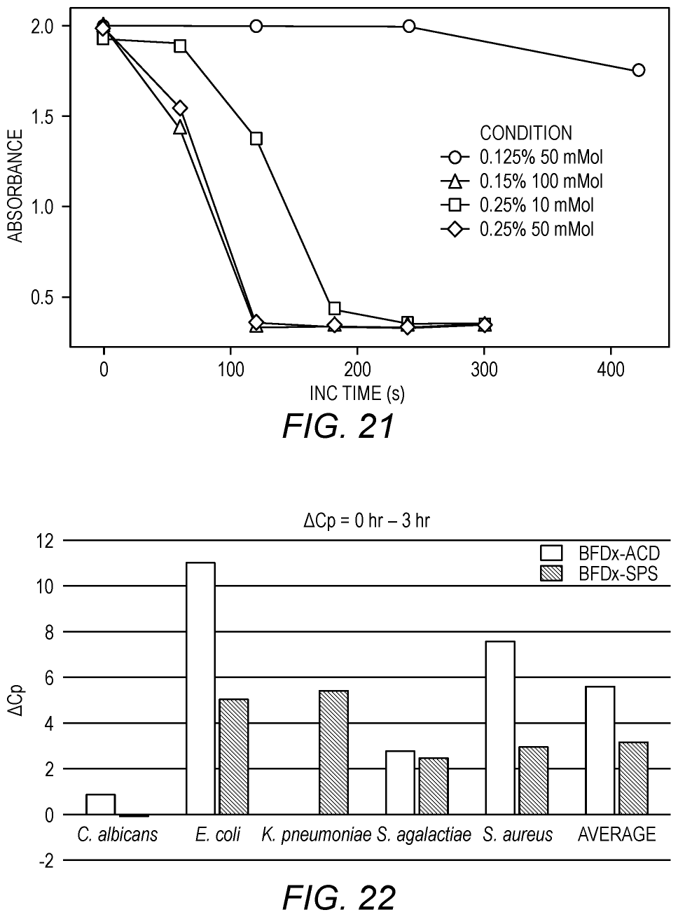

FIG. 22 illustrates another experiment comparing post-lysis and centrifugation culture for organisms recovered from ACD anticoagulant blood and SPS anticoagulant blood. This study was conducted for C. albicans, E. coli, K. pneumoniae, S. agalactiae, and S. aureus. After lysis with LB20 and recovery of cells by centrifugation, 150 uL of BHI broth was mixed with the pellet from the centrifugal concentrator and incubated at 37° C. for 0 hr or 3 hr. The Cp improvements shown in FIG. 22 represents the average reduction in Cp (i.e., shorter time to detection) observed for the 3 hr culture relative to the sample cultured for 0 hr.

In this study, cells from ACD blood showed about a 5.5 Cp improvement after 3 hrs or culture at 37° C. and cells recovered from SPS blood showed about a 3 Cp improvement after 3 hrs or culture at 37° C. The improvements for E. coli and S. aureus were most dramatic. C. albicans, which grows more slowly than bacteria, improved by only about 1 Cp for ACD blood and actually performed slightly worse for SPS blood. In this study, no ACD performance data was obtained for K. pneumoniae. This study illustrates that certain anticoagulants may affect the growth and culturability for some organisms. For all organisms in this study, ACD appeared to be less detrimental to growth and culturability as compared to SPS.

### Example 3—Flow Through Lysis, Culture, and Volume Reduction Systems

In addition to or in combination with the other devices discussed herein, flow through systems can be used for cell lysis, culture, and volume reduction. A schematic of one example of such a system 1500 is illustrated in FIG. 15. Flow through system 1500 includes three adjacent buffer chambers 1502, 1506, and 1510 that contain, respectively, a first buffer 1504, and second buffer 1508, and a third buffer 1512. System 1500 further includes a channel 1514 (e.g., a tube or an open trough of buffer exchange membrane that is in contact with the buffers 1504, 1508, and 1512 in each of buffer chambers 1502, 1506, and 1510. In one embodiment, the first buffer 1504, second buffer 1508, and third buffer 1512 may be comprised of a selective lysis buffer, a media or nutrient broth for culturing microbial cells (e.g., a nutrient broth for culturing bacterial organisms, fungal organisms, or a broth suited for culturing both bacterial and fungal organisms), and a hypertonic solution/media to decrease sample volume. In one embodiment, system 1500 may also include a temperature control system (not shown) that can adjust and control the temperature of the first buffer 1504, second buffer 1508, and third buffer 1512 (either individually or as a group) to enhance, for example, selective lysis, culturing of microbial organisms, and volume reduction. For example, selective lysis may be carried out at room temperature, culturing of microbial organisms may be carried out at 32-37° C., and volume reduction may be carried out at 4° C. A sample (e.g., a whole blood sample) disposed in channel 1514 may be selectively exposed to each of buffers 1504, 1508, and 1512, in any given order or to more than one buffer chamber at a time, to accomplish, for example, blood cell lysis, culturing of microbial cells, and sample volume reduction/concentration.

In one embodiment, a whole blood sample that includes microbial cells (e.g., a whole blood sample from a subject suspected of having sepsis) may be added to channel 1514 so that the blood sample can be selectively exposed to each of buffers 1504, 1508, and 1512. Buffer exchange membranes are widely known in the art. Suitable buffer exchange membranes may be choses such that blood cell debris, hemoglobin, and other products of blood cell lysis may diffuse through the membrane while microbial cells are retained. For example, the buffer exchange membrane may be a dialysis membrane. Dialysis membranes membranes are produced and characterized as having differing molecular-weight cutoffs (MWCO) ranging, for example, from 1 kilodalton (kDa) to about 1 MDa (i.e., 1 megadalton, or about 1000,000 Da). The MWCO determination is the result of the number and average size of the pores created during the production of the dialysis membrane. The MWCO typically refers to the smallest average molecular mass of a standard molecule that will not effectively diffuse across the membrane upon extended dialysis. It is important to note, however, that the MWCO of a membrane is not a sharply defined value. Molecules with mass near the MWCO of the membrane will diffuse across the membrane slower than molecules significantly smaller than the MWCO. In order for a molecule to rapidly diffuse across a membrane, it typically needs to be at least 20-50 times smaller than the membrane's MWCO rating. Dialysis tubing for laboratory use is typically made of a film of regenerated cellulose or cellulose ester. However; dialysis membranes made of polysulfone, polyethersulfone (PES), etched polycarbonate, or collagen are also extensively used for specific medical, food, or water treatment applications.

Because microbial cells are relatively large and cell debris is relatively small, channel 1514 may also be fabricated from a filtration membrane material. Membrane materials designed to filter bacteria and larger cells out of solution are well known in the art. For example, a filtration membrane having a nominal pore size of 0.25-1 μm (e.g., 0.5 μm) may be used to retain microbial cells in channel 1514 while allowing the sample in channel 1514 to rapidly exchange with buffers 1504, 1508, and 1512.

Referring to FIG. 15, a sample (e.g., a whole blood sample) disposed in channel 1514 may first be exposed to buffer 1504 in chamber 1502, as shown at 1516 in FIG. 15A. Then the sample may be moved so that it is exposed to buffer 1508 in chamber 1506, as shown at 1518 in FIG. 15B, and then the sample may be moved so that it is exposed to buffer 1512 in chamber 1510, as shown at 1520 in FIG. 15C. While FIG. 15A-15C shows the sample being moved sequentially from one buffer chamber to another so that the sample is exposed to each buffer in turn, one will appreciate that the sample can be moved back and forth so that it is exposed to buffers more than once, exposed to more than one buffer at a time, etc. Likewise, buffers 1504, 1508, and 1512 may be arranged in any given order. Table 11 illustrates some of the options.

TABLE 11

| Buffer 1504 | Buffer 1508 | Buffer 1512 |
|---|---|---|
| Selective Lysis Buffer | Culture Media | Hypertonic Solution |
| Culture Media | Selective Lysis Buffer | Hypertonic Solution |
| Culture Media | Hypertonic Solution | Selective Lysis Buffer |
| Selective Lysis Buffer | Hypertonic Solution | Culture Media |
| Hypertonic Solution | Selective Lysis Buffer | Culture Media |
| Hypertonic Solution | Culture Media | Selective Lysis Buffer |

And while this example discusses using selective lysis buffer, culture media, and hypertonic solution for lysis, culturing microbial cells, and sample volume reduction, respectively, persons skilled in the art will recognize that these buffers are merely illustrative and that other buffers may be used with system 1500. Likewise, while system 1500 includes three buffer tanks, this is merely illustrative. Alternative versions of the system 1500 may include more or fewer buffers. In addition, while channel 1514 is shown as a linear channel, this is merely illustrative. Channel 1514 may, for example, include a circuitous flow path or other modifications known in the art to maximize the surface area of the sample that is exposed to the buffers.

In one embodiment, the hypertonic media may be sufficient to concentrate microbes in the sample to permit identification (e.g., by PCR techniques, whole genome sequencing, or molecular AST, phenotypic techniques, proteomic techniques, and microscopic techniques). In other embodiments, a filtration technique may be used for concentration/volume reduction. Filtration may be performed before or after one or more of exposure of the sample to selective lysis buffer, culture media, and hypertonic solution. In another embodiment, a centrifugation technique may be used to concentrate microbes in the sample. Centrifugation may be performed before or after one or more of exposure of the sample to selective lysis buffer, culture media, and hypertonic solution.

### Example 4—Filtration Techniques

In some embodiments described herein, separation of microbial cells from their milieu (e.g., separation of bacterial and/or fungal cells from a whole blood sample) can be carried out by filtration. Filtration techniques may be designed to retain or pass through selected cells or cell sizes. For example, blood cells (e.g., red blood cells, white blood cells, platelets, etc.) may be trapped while microbial cells may be passed through, microbial cells may be trapped, or a combination of filtration media may be used to selectively trap large and small cells at different stages of a filtration apparatus. Differential filtration techniques may also be employed to separate larger and smaller cells into different fractions. For example, filtration membranes having different nominal pore sizes may be stacked (or used in a series of separate containers) to pass and/or trap cells having selected size ranges. Flow cytometry is also a well known technique that is capable of sorting cells by size. Cells may also be trapped or enriched by active filtration techniques. For example, most cell types have specific surface factors (e.g., proteins) that can be used for affinity purification by techniques well known in the art.

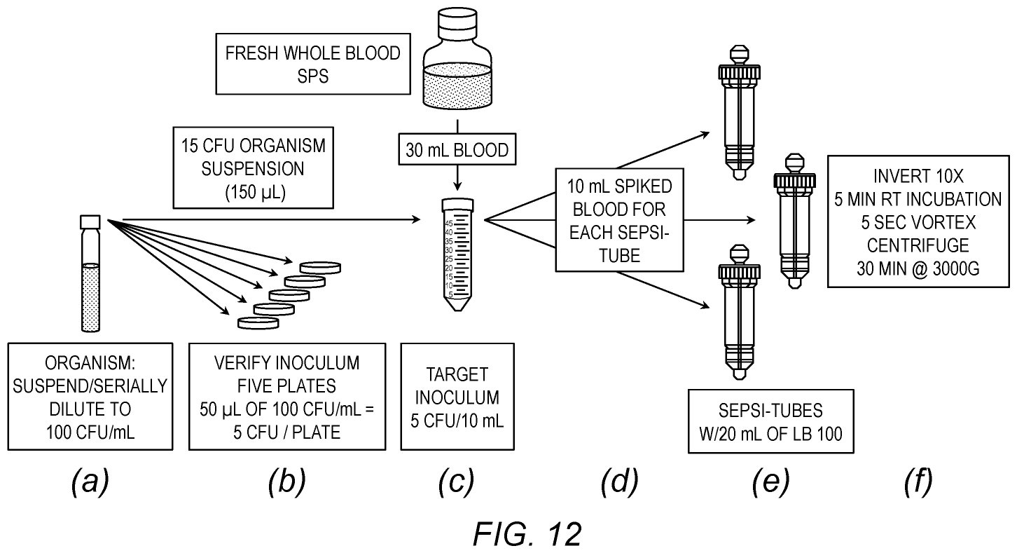

An example of a differential filtration system is illustrated in FIG. 16. A whole blood sample from a subject suspected of having sepsis may be enriched for microbial cells by first passing the whole blood sample through a large filter having a pore size of 8-15 μm (e.g., 10 μm) to filter out large cells like white blood cells (WBCs) and some red blood cells. The microbial cells would flow through the first filter. In one embodiment, a sub-lytic level of Brij detergent (e.g., <0.1%) could be used to ensure that any microbial cells adhered to the outside of WBCs are released to reduce trapping of microbial cells on WBC that are trapped by the filter. Other detergents suitably may have other sub-lytic concentration levels—in general, 0.1%-1%. A second filter with a smaller pore size (e.g., 5 μm) may be used in tandem to remove more human cells while enriching for microbial cells in the filtrate. A final filter having a pore size of less than 1 μm (e.g., 0.45 μm) may be used to capture all microbial cells and significantly reduce the volume of the sample. Filter concentrated microbial cells may be used directly for identification and diagnosis (e.g., molecular identification with FilmArray, imaging, optical fluorescence of metabolic process, or metabolic consumption, conductivity, pH, etc.), the sample may be cultured (e.g., for 1 to 3 hrs) to enrich the numbers of microbial cells in the sample, or they may be subjected to alkaline lysis to further remove animal cells (e.g., human cells), centrifugation, and molecular identification, as described herein.

In another embodiment, filtration may be used to recover cells after selective lysis with the alkaline/Brij buffer described here (i.e., alkaline lysis). However, it was found that proteinase K treatment was needed to reduce the viscosity of the sample prior to filtration. In this context, the alkaline/Brij selective lysis buffer was added to whole blood and incubated for 5 minutes. After 5 mins, 1 mL of 30 units/mL proteinase K was added and incubated for about 5 mins at RT. The lysate could then be filtered through a 0.45 μm filter. As in the previous example, filter concentrated microbial cells may be used directly for identification and diagnosis, they may be cultured (e.g., for 1 to 3 hrs) to enrich the numbers of microbial cells in the sample, etc.

In addition to the conventional filtration techniques described above, filtration techniques that use various types of structures selectively enrich certain cell types in a sample may be used. Such filtration techniques may be used in lieu of conventional filtration or in combination with conventional filtration to enrich or isolate microbial cells of interest from blood cells to reduce volumes and inhibitors. Such enriched or isolated microbial cells may be subjected to culture calls (similar to conventional blood culture, but likely more rapid because the microbial cells in the sample are enriched), FilmArray identification, or other interrogation techniques. An additional desire is to confirm that bacterial cells are likely present to make the process more economical for the customer (e.g., a less expensive check like imaging, optical fluorescence of metabolic process, or metabolic consumption, conductivity, or pH).

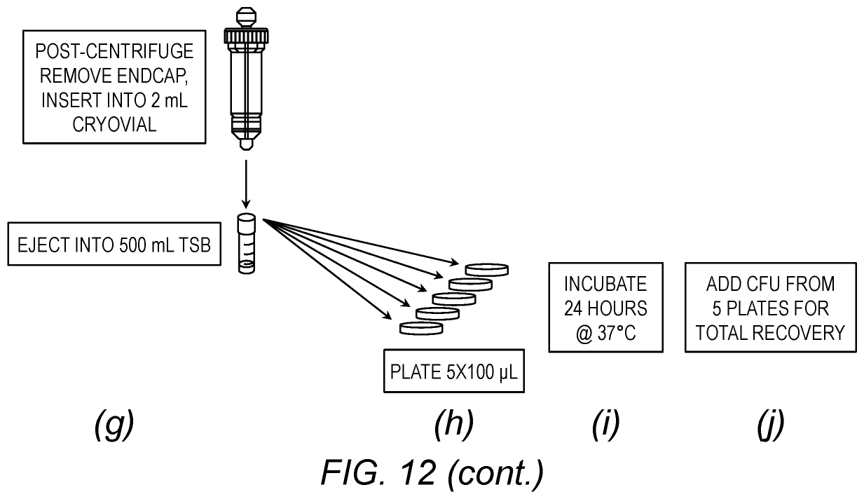

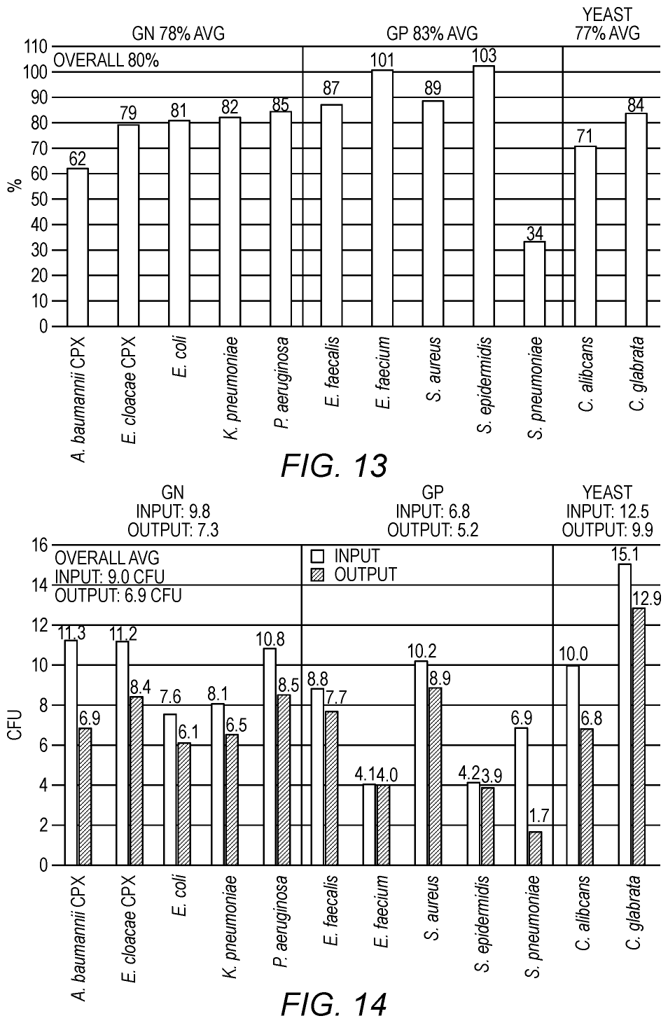

Various filtration techniques that can be used to enrich certain cells are illustrated in FIGS. 17-20. FIG. 17A illustrates a weir filter, 17B illustrates a micropillar filter, and 17C illustrates a cross-flow filter. Differential flow of larger and smaller cells around these structures can be used to separate smaller cells from larger cells. FIG. 18 schematically illustrates different types of pillar filters (18A) polygonal, (18B) U-shaped, and (18C) butterfly-shaped micropillar geometries. Larger cells are immobilized in trapping structures, while smaller cells pass through. FIG. 18B also schematically illustrates the concept that the micopillars may be formed with structural features (shapes, pockets, etc.) to selectively retard passage of certain cells through the micropillar structure.

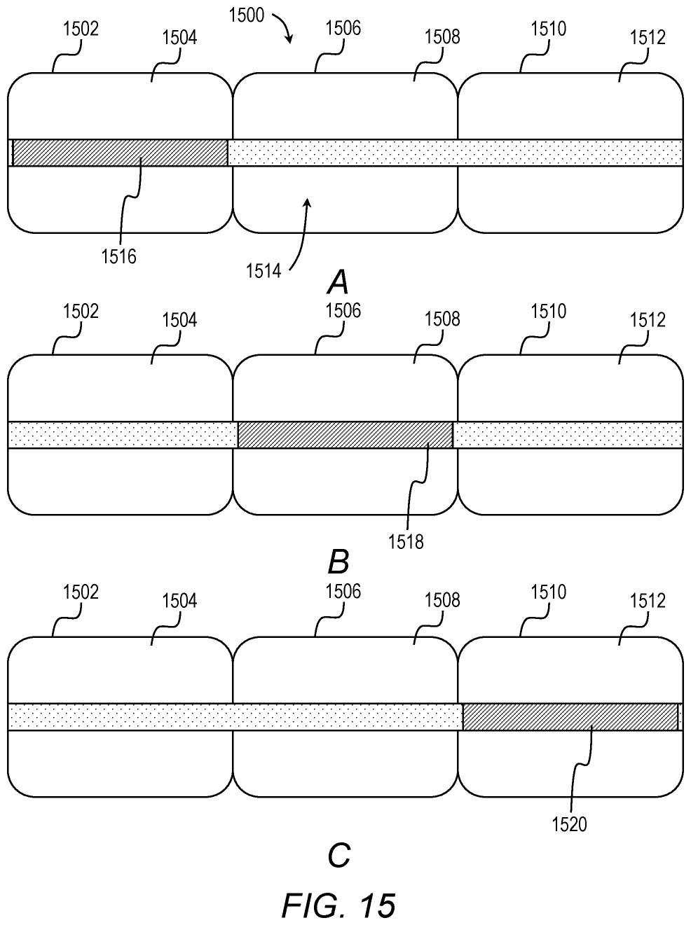

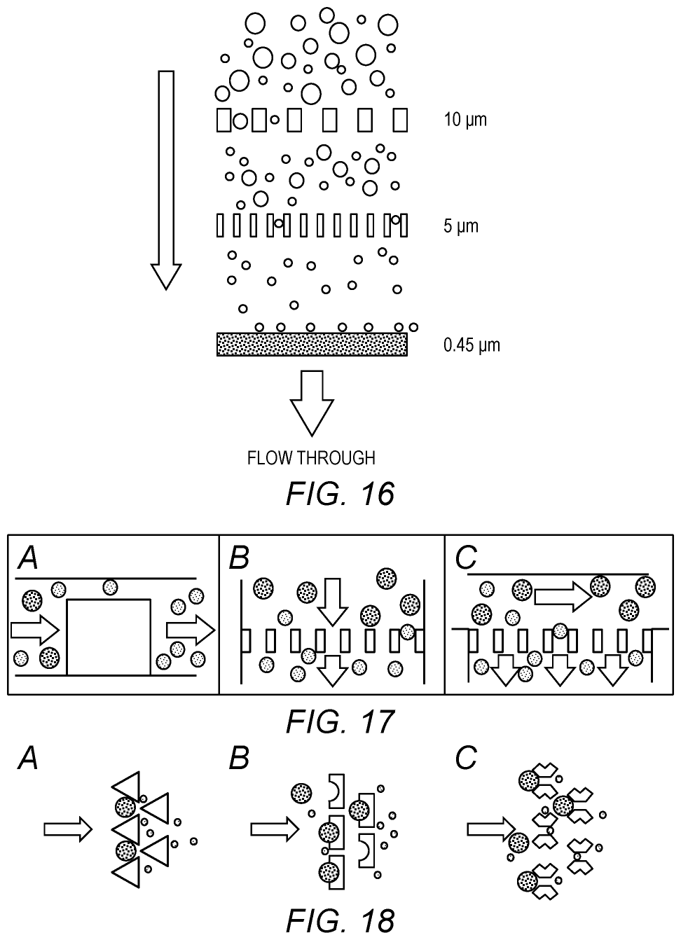

While FIGS. 17 and 18 shows only one set of each of these structures, such structures (and flow directions) may be used in series and in combination to achieve high levels of separation. FIGS. 19 and 20 illustrate this principle. FIG. 19 illustrates separation of large and small cells in a structure with an array of micopillars and cross-flows of buffer and cell suspension. The FIG. 19 structure separates large and small cells by deterministic lateral displacement. Large cells migrate away from the small cells in the streamline due to the engineered size and spacing of the microposts in the fluidic channel. FIG. 20 schematically illustrates the concentration large and small cells by migration along an oval-shaped filter unit. The filter unit achieves simultaneous separation of large cells, which are larger than gaps, and small cells, which are smaller than the gaps. Rolling along the pillars at relatively low velocities, which is induced by the filtrate shear layer, helps to prevent the clogging of large particles. The systems in FIGS. 19 and 20 are examples of systems that may be used for removal of lysate, buffer exchange, and addition of culture media for growth in one system. That is, lysate may be flowed in to start separation, buffer may be added to flush away lysate, and culture media may be added. A filtration concentrator, a microfluidic concentrator, a dielectrophoretic concentrator, FACS (fluorescence activated cell sorting), or other similar devices may suitably be used in addition to or in lieu of the centrifugal concentrator described herein. A benefit of the selective lysis suitably may be that it could simplify the filtration concentrator mechanisms or enable them to process more volume before fouling.

Workflows that may include one or more of the chemistry, filtration, centrifugation, and identification (e.g., molecular identification or another technique such as, but not limited to imaging, optical fluorescence of metabolic process, or metabolic consumption, conductivity, or pH).
Path 1: Selective lysis with alkaline/Brij buffer, transfer lysate to a microfluidic chip for enrichment and culture, FilmArray for detection and identification
1. Selectively Lyse human cells with alkaline/Brij selective lysis buffer
2. Enrich for microbial cells with one or more of centrifugation, filtration device, or microfluidic chip design
a. Sorting technology (active (e.g., flow cytometry) or passive (weir filtration, micropillar filtration, or a combination thereof)
b. Trapping technology (active or passive)
c. Filtration technology (usually passive but possibly active)
3. Flush with culture media for growth
a. In some embodiments, centrifugation, filtration, microfluidic separation, or a combination thereof may be used for removal of lysate, buffer exchange, and addition of culture media for growth in one system
b. In some embodiments, additional sensing technology for positive detection of microbial cells may be added
i. Imaging
ii. Optical fluorescence of metabolic process, or metabolic consumption
iii. Conductivity
iv. pH
v. micro-resonators
vi. Dielectrophoresis
vii. capacitance sensing
viii. SPR
ix. FLIR
4. Release cells from the microfluidic device for FilmArray analysis, culture, or other confirmatory processes.
Path 2: Use microfluidic chip/selective filtration for both sorting/enrichment and culture, FilmArray, or other confirmatory processes for detection
1. Selectively sort microbial cells from human blood cells
a. Active Sorting
i. Flow cytometry (fluorescence activation or optical detection)
ii. Dielectrophoresis (DEP) sorting
iii. Pneumatic sorting
b. Passive Sorting
i. Size sorting
ii. Inertial sorting
iii. Dielectric trapping
iv. Selective protein adhesion process
v. acoustic trapping
vi. viscoelastic (or cell stiffness) sorting in a shear gradient
2. Flush with culture media for growth
a. In some embodiments, centrifugation, filtration, microfluidic separation, or a combination thereof may be used for removal of lysate, buffer exchange, and addition of culture media for growth in one system
b. In some embodiments, additional sensing technology for positive detection of microbial cells may be added
i. Imaging
ii. Optical fluorescence of metabolic process, or metabolic consumption
iii. Conductivity
iv. pH
v. micro-resonators
vi. Dielectrophoresis
vii. capacitance sensing
viii. SPR
ix. FUR
4. Release cells from the microfluidic device for FilmArray analysis, culture, or other confirmatory processes.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached invention disclosure for purposes of illustrating the invention, it will be apparent to those skilled in the art that various changes in the methods and apparatus disclosed herein may be made without departing from the scope of the invention, which is defined in the appended claims. All changes which come within the meaning and range of equivalency of the claims are to be embraced within their scope.

What is claimed is:

1. A method of isolating and identifying a microorganism, comprising:
(a) providing a volume of a blood sample suspected of containing the microorganism;
(b) mixing the blood sample with a differential lysis buffer to yield a lysate, wherein the lysate comprises lysed blood cells and unlysed microorganism;
(c) concentrating the microorganism from the lysate;
(d) adding the microorganism to a device that includes one or more reagents needed for identifying the microorganism; and
(e) responsive to adding the microorganism to a device, performing an assay with the one or more reagents and identifying the microorganism present in the blood sample,
wherein the microorganism, if present, is concentrated in a range of 25 to 100 fold relative to the volume of the provided blood sample,
wherein the microorganism, if present, has a concentration in a range of about <1 CFU/ml to about 20 CFU/ml in the provided blood sample, and
wherein steps (a)-(e) can be completed in less than about 120 minutes, preferably in less than about 90 minutes.

2. The method of claim 1, wherein steps (a)-(c) can be completed in a time range of about 10 to 20 minutes.

3. The method of claim 1, wherein steps (d) and (e) can be completed in a time range of less than 4 hrs, preferably less than 3 hrs, preferably less than 2 hrs, or more preferably less than 1 hr.

4. The method of claim 1, wherein the microorganism is a bacterium or fungal organism associated with a bloodborne infection.

5. The method of claim 1, wherein the identifying includes one or more of a molecular test, a phenotypic test, a proteomic test, an optical test, or a culture-based test.

6. The method of claim 1, wherein the identifying includes steps of isolating from the microorganism one or more nucleic acids characteristic of the microorganism, and analyzing the one or more nucleic acids to identify the microorganism present in the blood sample, preferably wherein the identifying includes steps of isolating from the microorganism one or more nucleic acids characteristic of the microorganism, and analyzing the one or more nucleic acids to identify the microorganism present in the blood sample, more preferably wherein the identifying further comprises amplifying one or more nucleic acids and then detecting the one or more amplified nucleic acids, and even more preferably wherein the detecting the one or more amplified nucleic acids includes use of one or more of a dsDNA binding dye, real-time PCR, a post-amplification nucleic acid melting step, a nucleic acid sequencing step, a labeled DNA binding probe, or an unlabeled probe.

7. (canceled)

8. (canceled)

9. (canceled)

10. The method of claim 1, further comprising performing a culture step on the concentrated microorganism in culture media to increase concentration of the microorganism and then performing the steps of identifying, wherein the culture step is performed for 4 hrs or less, 3 hrs or less, or 2 hrs or less, preferably 3 hrs or less.

11. The method of claim 1, wherein the differential lysis buffer comprises a buffering substance, a nonionic surfactant, a salt, and a pH range of about 10-11 prior to mixing the blood sample with the differential lysis buffer and wherein the lysate has a pH about 1.5 to 2.5 pH units below the pH buffering range of the buffering substance.

12. The method of claim 11, wherein the lysate has a pH of about 7.0 to 8.0 after mixing the blood sample and the differential lysis buffer.

13. The method of claim 11, wherein the buffering substance is selected from the group consisting of CABS, CAPS, CAPS, CHES, and combinations thereof, and wherein the buffering substance is preferably CAPS.

14. (canceled)

15. The method of claim 11, wherein the nonionic surfactant is a polyoxyethylene (POE) ether, preferably one or more of Arlasolve 200 (aka, Poly(Oxy-1,2-Ethanediyl)), Brij O10, and nonaethylene glycol monododecyl ether (aka, Brij 35).

16. The method of claim 11, wherein the nonionic surfactant is selected from the group consisting of Triton X-114, NP-40, Arlasolve 200, Brij O10 (aka, Brij 96/97), octyl β-D-glucopyranoside, a saponin, nonaethylene glycol monododecyl ether (aka, Brij 35), and combinations thereof.

17. The method of claim 1, wherein concentrating the microorganism from the lysate includes centrifugation, and the concentrating further comprises recovering a pellet fraction comprising the microorganism from a supernatant fraction comprising a lysed blood fraction.

18. (canceled)

19. (canceled)

20. (canceled)

21. The method of claim 1, wherein the method does not include one or more of a culture step prior to mixing the blood sample with the differential lysis buffer, or a DNase step to digest genomic DNA in the lysate.

22. (canceled)

23. (canceled)

24. (canceled)

25. (canceled)

26. (canceled)

27. (canceled)

28. A method of concentrating and identifying a microorganism from blood, comprising:
(a) providing a blood sample known to contain or that may contain the microorganism;
(b) mixing the blood sample with a differential lysis buffer to form a lysate comprising lysed blood cells and unlysed microorganism, wherein the differential lysis buffer comprises a buffering substance having a useful pH buffering range of about 8.6-11.4, a nonionic surfactant, a salt, and a pH range of about 10-11 prior to mixing the blood sample with the differential lysis buffer, wherein the lysate has a concentration of the buffering substance of about 10 mM and a pH about 7.0 to 8.0;
(c) concentrating the microorganism from the lysate, wherein the microorganism is concentrated in a range of 25 to 100 fold relative to a starting volume of the provided blood sample; and
(d) identifying the microorganism present in the blood sample, wherein the identifying is accomplished in 4 hrs or less, 3 hrs or less, 2 hrs or less, or, preferably, 1 hr or less.

29. The method of claim 28, wherein the identifying includes one or more of a molecular test, a phenotypic test, a proteomic test, an optical test, or a culture-based test.

30. (canceled)

31. (canceled)

32. The method of claim 28, wherein the nonionic surfactant is selected from the group consisting of Triton X-114, NP-40, Arlasolve 200, Brij O10 (aka, Brij 96/97), octyl β-D-glucopyranoside, a saponin, nonaethylene glycol monododecyl ether (aka, Brij 35), and combinations thereof.

33. The method of claim 28, wherein the buffering substance is selected from the group consisting of CABS, CAPS, CAPSO, CHES, and combinations thereof.

34. (canceled)

35. (canceled)

36. (canceled)

37. The method of claim 28, wherein the time to yield the lysate is in a range of about 2 to 10 minutes, preferably about 5 minutes, wherein steps (a)-(e) can be completed in less than about 120 minutes, preferably in less than about 90 minutes, wherein steps (a)-(c) can be completed in a time range of about 10 to 20 minutes, wherein steps (d) and (e) can be completed in a time range of less than 4 hrs, preferably less than 3 hrs, preferably less than 2 hrs, or more preferably less than 1 hr, and wherein steps (b)-(e) can be completed in about 20-75 minutes.

38. (canceled)

39. (canceled)

40. (canceled)

41. A composition, comprising
a whole blood sample known to contain or that may contain a microorganism; and
a differential lysis buffer that is combined with the blood sample, the differential lysis buffer comprising an aqueous medium, a buffering substance having a useful pH buffering range of about 8.6-11.4 and a pKa at 25° C. in a range of about 9.5 to about 10.7, a nonionic surfactant, a salt, and a pH range of about 10-11 prior to mixing the blood sample with the differential lysis buffer,
wherein the composition has a concentration of the buffering substance of about 10 mM and a pH of about 7.0 to 8.0.

42. (canceled)

43. The composition of claim 41, wherein the nonionic surfactant is selected from the group consisting of Triton X-114, NP-40, Arlasolve 200, Brij O10 (aka, Brij 96/97), octyl β-D-glucopyranoside, a saponin, nonaethylene glycol monododecyl ether (C12E9, polidocenol), and combinations thereof.

44. The composition of claim 41, wherein the buffering substance is selected from the group consisting of CABS, CAPS, CAPSO, CHES, and combinations thereof.

45. (canceled)

46. (canceled)

47. (canceled)

48. The composition of claim 41, consisting essentially of
the whole blood sample known to contain or that may contain microorganism; and
the differential lysis buffer comprising CAPS as the buffering substance and a pH of about 10-11 prior to mixing the whole blood sample with the differential lysis buffer, and the nonionic surfactant,
wherein the composition has a pH about 7.0 to 8.0 after mixing the whole blood sample with the differential lysis buffer.

49. The method of claim 1, wherein steps (b)-(e) can be completed in about 20-75 minutes.
